(19) United States
(12) Patent Application Publication
(10) Pub. No.: US 2025/0243270 A1
(43) Pub. Date: Jul. 31, 2025

(54) PREDICTION AND TREATMENT OF FAST PROGRESSING RENAL DISEASE

(71) Applicant: Bird Rock Bio Sub, Inc., San Diego, CA (US)

(72) Inventors: Anke Kretz-Rommel, San Diego, CA (US); Paul Grayson, La Jolla, CA (US)

(21) Appl. No.: 18/853,338

(22) PCT Filed: Apr. 3, 2023
(86) PCT No.: PCT/US2023/065265

§ 371 Date: Oct. 1, 2024

Related U.S. Application Data
(60) Provisional application No. 63/327,165, filed on Apr. 4, 2022.

(51) Int. Cl. C07K 16/28; A61K 39/00; A61P 13/12; G01N 33/53

(52) U.S. Cl. CPC C07K 16/28; A61P 13/12; G01N 33/5308; A61K 2039/505; C07K 2317/24; G01N 2800/347

(57) ABSTRACT

The disclosure is directed to a method for predicting whether a patient is at risk for developing Fast Progressing Renal Disease (FPRD) and treating patients to avoid such risk.

### FIELD OF THE DISCLOSURE

The disclosure is directed to a method for predicting whether a patient is at risk for developing Fast Progressing Renal Disease (FPRD) and treating patients to avoid such risk.

### BACKGROUND

Chronic kidney disease (CKD) is highly prevalent but identification of patients at high risk for fast CKD progression, or fast progressing renal disease (FPRD) before reaching end-stage renal disease in the short-term has been challenging. Whether factors associated with fast progression vary by diabetes status is also not well understood.

Accordingly, there is a need in the art to identify when patients will develop fast progressing renal disease so that early therapeutic intervention is possible.

### SUMMARY

In one aspect the disclosure is directed to a method of predicting whether a patient is at risk for developing Fast Progressing Renal Disease (FPRD). The method includes determining the concentration of one or more endocannabinoids selected from Arachidonoyl Ethanolamide (AE), D-erythro-Sphingosine (DeSph), Di-homo-γ-Linolenoyl Ethanolamide (DhyLE), Docosatetraeonoyl Ethanolamide (DE), Linoleoyl Ethanolamide (LE), Palmitoyl Ethanolamide (PE) and Palmitoleoyl Ethanolamine (POEA) in a whole blood, serum or plasma sample from the patient and correlating the concentration of POEA in the sample to the likelihood that the patient will develop FPRD. The patient may be suffering from diabetes or pre-diabetes, and the diabetes may be severe insulin resistant diabetes (SIRD).

In various aspects of the disclosure, the correlating includes determining that the patient will develop FPRD when the level of POEA in the sample exceeds about 0.1 mg, or the correlating includes determine that the patient will develop FPRD when the level of POEA in the sample exceeds about the 90th percentile of FPRD in a population of diabetic patients. In addition, the correlating may include determining that the patient will develop FPRD when the level of POEA in the sample exceeds about the 90th percentile of FPRD in a population of patients suffering from pre-diabetes. Still further, the correlating may include predicting that the patient will develop FPRD in 1-5 years when the level of POEA in the sample exceeds about 0.1 mg.

Another aspect of the disclosure includes a method of identifying a diabetes patient that will develop FPRD. The method includes determining the concentration of palmitoleoyl ethanolamide (POEA) in a whole blood, serum or plasma sample from the patient and determining that that the patient will develop FPRD when the concentration of POEA in the sample exceeds about the 90th percentile of the POEA concentration in a population of diabetes patients.

Still further, aspects of the disclosure include a method of treating a patient that has an increased risk for FPRD. The method includes:
(a) determining the concentration of palmitoylethanolamide (POEA) in a whole blood, serum or plasma sample from the patient,
(b) determining that that the patient has an increased risk of developing FPRD when the concentration of POEA in the sample exceeds about the 90th percentile of the POEA concentration in a population of diabetes patients; and
(c) treating the patient with an antagonist of cannabinoid 1 (CB1) receptor.

Yet another aspect of the disclosure includes method of treating a patient that has SIRD to avoid progression to FPRD. The method includes
(a) determining the concentration of palmitoylethanolamide (POEA) in a whole blood, serum or plasma sample from the patient,
(b) determining an expected onset of FPRD base upon the concentration of POEA in the sample; and
(c) treating the patient with an antagonist of cannabinoid 1 (CB1) receptor.

In the various aspects of the disclosure, treatment with an agonist of CB1 receptor includes treatment with an antibody or antigen binding fragment that binds to CB1 receptor.

### BRIEF DESCRIPTION OF THE FIGURES

The following detailed description of the embodiments of the present disclosure can be read in conjunction with the following drawings.

FIG. 1 shows a cohort overview in which 526 age-, and sex-matched patients were stratified into severe insulin-resistant diabetes (SIRD; with HOMA-IR>5) and non-SIRD (HOMA-IR<5) groups for assessment of endocannabinoid levels.

FIG. 2 shows chemical structures of several of the 13 endocannabinoids measured as described herein.

FIGS. 3A-3C show the distribution of endocannabinoids in the total cohort wherein the y-axis represents the number of patients and the x-axis represents the level of endocannabinoid in relative units. Putative Endocannabinoid 3 was later confirmed to be Palmitoleoyl Ethanolamide (POEA).

FIG. 4 shows that certain endocannabinoids are significantly elevated in severe insulin-resistant diabetes (SIRD) patients over age-, sex- and BMI-matched controls without SIRD (“Non-SIRD”; p≤0.05).

FIG. 5 shows associations among certain endocannabinoids elevated in SIRD patients with HOMA-IR, glucose, age, BMI and baseline glomerular filtration rates (GFR). The presence of a circle at the intersection between a SIRD factor and a specific endocannabinoid indicates a significant association, with larger circles indicating increased significance. Shading indicates the magnitude of the effect (darker shading indicating a greater effect). While not apparent in the grey scale figures, color indicates the direction of the correlation, red being positive correlation and blue being negative correlation.

FIG. 6 shows that GFRs decline modestly faster over time in patients with SIRD as compared to those without SIRD (left panel) and that GFR declines significantly faster in patients with FPRD as compared to those without FPRD (right panel).

FIG. 7 shows that while most endocannabinoids tested (see FIGS. 3A to 3C) demonstrated some level of association with FPRD, only palmitoleoyl ethanolamide (POEA) was predictive of FPRD.

FIG. 8 shows the relationship between FPRD and other aspects of kidney disease, including glomerular filtration rate (GFR), Homeostatic Model Assessment of Insulin Resistance (HOMA-IR), proteinuria, and combinations thereof. Both p-value and hazard ratio calculations show that FPRD is independent of these factors.

FIG. 9A shows a Kaplan-Meier curve of the incidence of FPRD in a population of SIRD patients in which the population (n=526) was divided into two groups, one comprising the top 10% of patients (n=53) stratified by POEA concentration and the other comprising the lower 90% of POEA concentration (n=473). Patients with highly elevated POEA were significantly more likely to develop FPRD (p=0.0021) than those with lower POEA concentrations. FIG. 9B shows an overview of the patient population used for the Kaplan-Meier curve.

FIGS. 10A-10B show triple quadrupole mass spectroscopy of putative endocannabinoid #3 confirming its identity as palmitoleoyl ethanolamide (POEA).

FIG. 11 shows that treatment of a patient with highly elevated POEA with nimacimab significantly decreased POEA levels at a dose of 0.6 mg/kg nimacimab, which is a peripheral antagonist of the CB1 receptor.

### DESCRIPTION

In general, the disclosure is directed to a method for determining, predicting and/or prognosing whether a patient is at risk for developing Fast Progressing Renal Disease (FPRD) and also directed to the treatment of such patients to delay, prevent, or ameliorate FPRD and associated symptoms. The method comprises determining the concentration of one or more cannabinoids in a whole blood, serum or plasma sample from the patient. Treatments include administering the patients identified in the diagnostic method of the disclosure with a therapeutically effective amount of an antagonist of the Cannabinoid 1 (CB1) receptor.

Before describing examples of embodiments of the disclosure in further detail, a number of terms are defined. The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components comprising more than one unit unless specifically stated otherwise.

“Fast Progressing Renal Disease” or “FPRD” refers to a condition in patients that is defined as a decline in glomerular filtration rate of greater than 5 unit/year and resulting in a GFR less than 60.

Cannabinoid 1 (CB1) receptor is a member of the G protein-coupled receptor (GPCR) superfamily. The CB1 receptor is expressed in the central nervous system (CNS), lungs, liver, adipose tissue and kidneys, and has been implicated in many human diseases including obesity, diabetes, fibrosis, liver diseases, cardiovascular disease, cancer, pain, MS spasticity, and glaucoma, among others. Cannabinoid 1 receptor may also be referred to herein as “CB1 receptor” or simply “CB1”.

As used herein, the term “antibody” refers to binding proteins having at least one antigen-binding domain and includes monoclonal antibodies fragments and/or variants thereof including recombinant polypeptides, fusion proteins, and immunoconjugates. Thus, the terms “antibody,” “antibody fragment,” and “antibody variant” are used interchangeably herein. Examples of antibody fragments of the disclosure include, but are not limited to, the Fab fragment, consisting of VL, VH, CL and CH1 domains; the Fc fragment, consisting of the VH and CH1 domains; the Fv fragment consisting of the VL and VH; the dAb fragment consisting of a VH domain; isolated CDR regions; F(ab′)₂ a bivalent fragment comprising two linked Fab fragments; and single chain Fv molecules (scFv). The CB1 receptor binding antibodies provided herein may be generated from any species including, but not limited to, mouse, rat, rabbit, primate, llama and human. The CB1 receptor binding antibodies may be chimeric, humanized, or fully human antibodies.

The antibodies and antigen-binding fragments thereof disclosed herein are specific for cannabinoid 1 (CB1) receptor. By “specific for” is meant that the antibodies and fragments thereof bind CB1 receptor with greater affinity (i.e., a lower binding affinity Kd value) than any other target. Thus, antibodies and fragments thereof that are selective for CB1 receptor bind CB1 receptor with greater affinity (i.e., a lower binding affinity Kd value) than any other cannabinoid receptor or any other GPCR or any other target. The antibodies and fragments or variants thereof may have a binding affinity Kd value for CB1 receptor in the range of about 0.01 nM to about 500 nM, about 0.02 nM to about 250 nM, about 0.02 to about 200 nM, about 0.05 to about 100 nM, about 0.05 to about 50 nM. The antibodies and fragments thereof may have a binding affinity Kd value for CB1 receptor of about 500 nM, about 250 nM, about 200 nM, about 150 nM, about 100 nM, about 75 nM, about 50 nM, about 25 nM, about 10 nM, about 5 nM, about 1 nM, about 500 μM, about 250 μM, about 100 μM, about 50 μM, or about 10 μM. The antibodies and fragments thereof may have a binding affinity Kd value for CB1 receptor of about 100 nM or less, about 75 nM or less, about 50 nM or less, about 10 nM or less, about 1 nM or less, about 500 μM or less, or about 100 μM or less.

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The Kabat definition is based on sequence variability. The IMGT unique numbering for all IG and TR V-regions of all species relies on the high conservation of the structure of the variable region (Lefranc, Mp et al., Dev comp. Immunol. 27:55-77, 2003). IMGT numbering, set up after aligning more than 5,000 sequences takes into account and combines the definition of the framework and CDRs. The Clothia definition is based on the location of the structural loop regions. The Contact definition (MacCallum et al.) is based on an analysis of the complex crystal structures and antibody-antigen interactions. The amino acid residues which encompass the CDRs as defined by each of the above cited references are set forth for comparison. In one embodiment disclosed herein, the term “CDR” is a CDR as defined by the Kabat definition. In another embodiment disclosed herein, the CDR is a CDR as defined by IMGT.

As used herein, the term “antagonist” refers to a compound that inhibits, diminishes or prevents the signaling activity of another compound at a receptor site and more generally refer to a compound that diminishes or prevents the activation and/or the signaling activity of a receptor.

As used herein, the terms “treatment” or “treating” refers to both therapeutic treatment and prophylactic or preventive measures. A subject in need of treatment is a subject that already has the disease or disorder as well as those that may develop the disease or disorder and in whom the object is to prevent, delay, or diminish the disease or disorder. The methods of “treatment” disclosed herein employ administration to a subject, an antibody or antigen binding fragment disclosed herein, for example, a subject determined to have or suspected to be at risk for developing FPRD in order to prevent, cure, delay, reduce the severity of, or ameliorate one or more symptoms or in order to prolong the survival of a subject beyond that expected in the absence of such treatment.

As used herein, the term “subject” denotes a mammal, such as, for example, a rodent, a feline, a canine, a primateor a a human.

A “therapeutically effective amount,” as used herein, refers to the amount of a compound or composition that is necessary to provide a therapeutic and/or preventative benefit to the subject. A therapeutically effective amount will vary depending upon the subject and disease condition being treated, the weight and age of the subject, the severity of the disease condition, the manner of administration and the like, which can readily be determined by one of ordinary skill in the art. Dosage regiments may be adjusted to provide the optimum therapeutic response. An effective amount is also one in which any toxic or detrimental effects (i.e., side effects) of an antibody or antigen binding fragment thereof are minimized or outweighed by the beneficial effects.

Turning now to the various aspects herein, the disclosure includes both methods of diagnosing (e.g., predicting, determining, and/or prognosing) whether a patient has or is at risk for developing Fast Progressing Renal Disease (FPRD) and treating patients that are identified by such diagnosis. The diagnostic aspect of the disclosure includes determining the concentration of one or more cannabinoids, which may be one or more of the following: Arachidonoyl Ethanolamide (AE), D-erythro-Sphingosine (DeSph), Di-homo-γ-Linolenoyl Ethanolamide (DhyLE), Docosatetraeonoyl Ethanolamide (DE), Linoleoyl Ethanolamide (LE), Palmitoyl Ethanolamide (PE), and Palmitoleoyl Ethanolamide (POEA) in a biological sample from the subject. In embodiments, the sample is a whole blood, serum or plasma sample from the patient. The sample may also be a urine sample. Patients that are identified by the method of diagnosis of the disclosure can be treated with a therapeutically effective amount of an antagonist of CB1 receptor. Many but not all patients suffering from or at risk for FPRD have diabetes, including severe insulin resistant diabetes (SIRD).

In various aspects of the disclosure, the cannabinoid that is measured in a patient sample (e.g., whole blood, plasma, serum or urine) is POEA. Patients that currently are not exhibiting symptoms for FPRD may be determined to be at risk for developing FPRD based upon the concentration or amount of a cannabinoid in the sample. For example, the concentration of FPRD in a sample may be correlated with the risk that the patient will develop FPRD. In some embodiments, a quantitative determination of cannabinoid in the sample may correlate to the level of risk that the patient will develop FPRD. In other embodiments, the patient is qualitatively determined to be at risk for developing FPRD when the level of the cannabinoid in the sample exceeds a threshold concentration. Still further, the method of the disclosure can predict how long before a patient will experience an onset of FPRD.

In some embodiments, the disclosure provides a method for determining a diagnosis, prognosis, or method of treatment in an individual that includes: i) determining a concentration of an endocannabinoid in a sample or determining the concentrations of an endocannabinoid in a series of samples from the individual, and ii) determining a diagnosis, prognosis, or method of treatment in the individual, based on the concentration in the sample, or on the concentrations in the series of samples. In some embodiments of the methods of the disclosure, step ii) includes an analysis such as comparing the concentration or series of concentrations to a normal value for the concentration of the endocannabinoid, comparing the concentration or series of concentrations to a predetermined threshold level, comparing the concentration or series of concentrations to a baseline value, and determining a rate of change of concentration for the series of concentrations.

In some embodiments of the methods of the disclosure, step ii) includes comparing the concentration of a cannabinoid in the sample with a predetermined threshold concentration, and determining a diagnosis, prognosis, or method of treatment if the sample concentration is greater than the threshold level. In some embodiments of the methods of the disclosure, the threshold concentration is determined by determining the a threshold percentile concentration of the cannabinoid in a population of individuals suffering from diabetes or pre-diabetes. The threshold concentration may be, for example, the 80th percentile, the 85th percentile, the 90th percentile, the 95th percentile, the 96th percentile, the 97th percentile, the 98th percentile or the 99th percentile of the level of the concentration of the endocannabinoid in a the population. In some embodiments of the methods of the disclosure, the endocannabinoid is selected from Arachidonoyl Ethanolamide (AE), D-erythro-Sphingosine (DeSph), Di-homo-γ-Linolenoyl Ethanolamide (DhyLE), Docosatetraeonoyl Ethanolamide (DE), Linoleoyl Ethanolamide (LE), Palmitoyl Ethanolamine (PE) and Palmitoleoyl Ethanolamide (POEA).

In some embodiments of the methods of the disclosure, the endocannabinoid is POEA and the threshold concentration is the 90th percentile of POEA in a population of patients suffering from diabetes or pre-diabetes. In some embodiments of the methods of the disclosure, the concentration or series of concentrations is determined at or near the time the individual presents to a health professional with symptoms of diabetes or SIRD, or the possibility thereof.

The method of the disclosure include correlating the level of a endocannabinoid to the risk that a patient, for example a patient suffering from diabetes or pre-diabetes, will develop FPRD in 1-5 years. For example, in one aspect of the disclosure, a patient is determined to be at risk for developing FPRD when the level of POEA in a plasma sample exceeds about 0.1 mg, about 1.5 mg or about 2.0 mg. In some embodiments the patient has diabetes or SIRD.

### Methods of Treatment

Methods of treatment according to the disclosure include treating individuals that have been identified to be at risk for developing FPRD. Treatments may include administering to a patient at risk a therapeutically effective amount of an antagonist of CB1 receptor sufficient to prevent FPRD, prolong the time to development of FPRD or, in patients already suffering from FPRD, slow the progression of FPRD.

In one aspect, the diagnosis according to the disclosure can occur 1-5 years prior to onset of FPRD, in particular, one year, two years, three years, four years, or five years prior to the beginning of a rapid decline in a patients GFR, which is indicative of FPRD. In some embodiments, treatment according to the disclosure can prolong the onset FPRD indefinitely, or up to 20 years, 10 years, 5, years, 4, years, 3 years, 2 years, or one year. In patients experiencing FPRD, slowing the progression of FPRD can include slowing the decline in a patients GFR but 1, 2, 3, 4, or 5 or more units per year.

Antagonists suitable for the treatments according to the disclosure include anti-CB1 receptor antibodies (or binding fragments thereof). A number of anti-CB1 antibodies are known in the art. See, e.g., WO2019211665A1, WO2014210205A1 and WO2017058771A1, each of which are incorporated by reference in its entirety. In addition, a number of anti-CB1 antibodies are described in the tables and disclosure following below. In one example, the antibody is nimacimab (Bird Rock Bio, Inc., La Jolla, California, USA), which is disclosed in WO2017058771A1 and further described herein.

In some embodiments of the disclosure, a patient determined to be at risk for FPRD is administered an anti-CB1 receptor antibody every week, every two weeks, every three weeks, every months, every six weeks, every two months, or every three months or more as necessary to prevent or delay the onset of FPRD. Patients suffering from FPRD may receive administration of the antibody on a similar schedule to delay the progressing of the disease including slowing or stopping the decline in a patient's GFR.

The anti-CB1 antibody can be delivered to a patient by in a variety of formulations and routes of administration as further described herein.

### Pharmaceutical Compositions

Administration of an anti-CB1 antibody according to the disclosure includes the administration of pharmaceutical compositions including an anti-CB1 antibody, or fragment thereof.

Methods of preparing and administering antibodies, or fragments thereof, disclosed herein to a subject are well known to or are readily determined by those skilled in the art. The route of administration of the antibodies, or fragments thereof, disclosed herein may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration can be used in certain embodiments. While all these forms of administration are clearly contemplated as being within the scope disclosed herein, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject disclosure, pharmaceutically acceptable carriers include, but are not limited to, 0.01-0.1M (e.g. 0.05M) phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will in an embodiment be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In certain embodiments, isotonic agents are included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

Effective doses of the antibodies, or fragments thereof, disclosed herein, for the treatment of SIRD or prevention of the progression of a diabetic patient to SIRD vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

Subjects can be administered such doses daily, on alternative days, weekly or according to any other schedule determined by empirical analysis. An exemplary treatment entails administration in multiple dosages over a prolonged period, for example, of at least six months or during the lifetime of the patient. Additional exemplary treatment regimens entail administration once per every two weeks or once a month or once every 3 to 6 months.

Intervals between single dosages can be, e.g., daily, weekly, monthly or yearly. Intervals can also be irregular as indicated by measuring blood levels of the endocannabinoid, or the therapeutic antibody in the patient. In some methods, dosage is adjusted to achieve a certain plasma antibody concentration, e.g., 1-1000 μg/ml or 25-300 μg/ml. Alternatively, antibodies, or fragments thereof, can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies. In one embodiment, the antibodies, or fragments thereof, disclosed herein can be administered in unconjugated form. In another embodiment, the antibodies disclosed herein can be administered multiple times in conjugated form. In still another embodiment, the antibodies, or fragments thereof, disclosed herein can be administered in unconjugated form, then in conjugated form, or vice versa.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 250 mg per dose, for example 0.1 mg, 1.0 mg, 10 mg, 50 mg, 100 mg, 150 mg, 200 mg or 250 mg per dose. In another embodiment, patients may receive about 0.1 to about 5.0 mg/kg, for example about 0.1 mg/kg, 0.2 mg/kg, 0.5 mg/kg, 1 mg/kg, 2 mg/kg, 3 mg/kg, 4 mg or 5 mg/kg. Particular examples include 0.6 mg/kg, 1.2 mg/kg and 2.5 mg/kg. Some patients continue to receive treatment for the rest of their lives.

In one embodiment, a subject can be treated with a nucleic acid molecule encoding a polypeptide disclosed herein (e.g., in a vector). Doses for nucleic acids encoding polypeptides range from about 10 ng to 1 g, 100 ng to 100 mg, 1 μg to 10 mg, or 30-300 ug DNA per patient. Doses for infectious viral vectors vary from 10-100, or more, virions per dose.

As previously discussed, the antibodies, or fragments thereof, disclosed herein can be administered in a pharmaceutically effective amount for the in vivo treatment of mammalian disorders. In this regard, it will be appreciated that the disclosed antibodies, or fragments thereof, will be formulated so as to facilitate administration and promote stability of the active agent. In certain embodiments, pharmaceutical compositions in accordance with the present disclosure comprise a pharmaceutically acceptable, non-toxic, sterile carrier such as physiological saline, non-toxic buffers, preservatives and the like. For the purposes of the instant application, a pharmaceutically effective amount of an antibody disclosed herein, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the polypeptide will in certain embodiments be capable of interacting with selected immunoreactive antigens on neoplastic or immunoreactive cells and provide for an increase in the death of those cells. Of course, the pharmaceutical compositions disclosed herein may be administered in single or multiple doses to provide for a pharmaceutically effective amount of the polypeptide.

In keeping with the scope of the present disclosure, the antibodies disclosed herein may be administered to a human or other animal in accordance with the aforementioned methods of treatment in an amount sufficient to produce a therapeutic or prophylactic effect. The polypeptides disclosed herein can be administered to such human or other animal in a conventional dosage form prepared by combining the antibody disclosed herein with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of polypeptides according to the present disclosure may prove to be particularly effective.

In one embodiment, the antibodies and binding fragments or variants thereof disclosed herein may be administered to the subject by at least one route selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intratympanic, intrauterine, intravesical, intravitreal, bolus, subconjunctival, vaginal, rectal, buccal, sublingual, intranasal, and transdermal.

### Methods of Detecting Endocannabinoids

The presence or amount of an endocannabinoid in patient sample can be accomplished by methods known to those of skill in the art. The disclosure herein is no limited to any particular detection method, including liquid chromatography mass spectrometry (LCMS). The Examples here describe detection of 23 endocannabinoids with LCMS from plasma samples and healthy controls. Once the presence or amount of an endocannabinoid has been detected, the value can be used to correlate endocannabinoid levels with SIRD status and prediction of a patient developing FPRD. In various forms of analysis, logistic regression can used and confounders accounted for (including, age, sex, and BMI). In one example, the association of endocannabinoids with fast progressive renal disease can be determined with Cox Linear regression was used and confounders (age, sex, BMI, proteinuria, HOMA-IR and GFR) accounted for. Statistical analysis may be conducted in R studio.

### Anti-CB1 Antibodies

In addition to the antibodies described herein, in some embodiments, CB1 receptor antibodies are generated by methods known to those of skill in the art, including recovering B cells from immunized mice and generating CB1 receptor antibody-producing hybridoma cells or the use of a phage library.

In some embodiments, the antibodies and fragments provided herein are chimeric antibodies or humanized antibodies. A “chimeric antibody” is an antibody having at least a portion of the heavy chain variable region and at least a portion of the light chain variable region derived from one species; and at least a portion of a constant region derived from another species. For example, in one embodiment, a chimeric antibody may comprise murine variable regions and a human constant region. A “humanized antibody” is an antibody containing complementarity determining regions (CDRs) that are derived from a non-human antibody; and framework regions as well as constant regions that are derived from a human antibody.

For example, the CB1 antibodies provided herein may comprise CDRs derived from one or more murine antibodies and human framework and constant regions. Thus, in one embodiment, the humanized antibody provided herein binds to the same epitope on CB1 as the murine antibody from which the antibody's CDRs are derived. Exemplary humanized antibodies are provided herein. Additional humanized CB1 antibodies comprising the heavy and light chain CDRs provided herein, or variants thereof, may be generated using any human framework sequence, and are also encompassed in the present disclosure. In one embodiment, framework sequences suitable for use in the present disclosure include those framework sequences that are structurally similar to the framework sequences provided herein. In some embodiments, human frameworks were selected based on homology between the parent antibody and the human germline VH and VK genes. Selected frameworks, in some embodiments, had the highest homology with the parent antibody VH and VK genes and also were predicted, based on computer modeling or other means, to support the CDR structure predicted to be presented by the parent antibody.

Further modifications in the framework regions may be made to improve the properties of the antibodies provided herein. Such further framework modifications may include chemical modifications; point mutations to reduce immunogenicity or remove T cell epitopes; or back mutation to the residue in the original germline sequence. In one embodiment of the present disclosure, the humanized antibodies and fragments thereof comprise a human framework and grafted CDRs provided herein, without further modifications to the variable region. Humanized antibodies that do not comprise a human framework backmutation are herein termed H0 (e.g., PIC4-H0). In another embodiment of the present disclosure, the humanized antibodies and fragments thereof comprise a human framework and grafted CDRs provided herein, wherein the amino acid at position 27 and/or and 28 of the heavy chain framework region 1 is backmutated. In a further embodiment, the amino acid at position 27 is backmutated from Gly (G) to Tyr (Y); and the amino acid at position 28 is backmutated from Thr (T) to Glu (E). Humanized antibodies having such mutations at positions 27 and 28 are herein described as “H2” or “H2 (YE)” (e.g., P1C4-H2 or P1C4-H2 (YE)). In another embodiment of the present disclosure, the humanized antibodies and fragments thereof comprise a human framework and grafted CDRs provided herein, wherein the amino acid at position 27 and/or and 28 of the heavy chain framework region 1 and the amino acid at position 60 and/or 61 of the heavy chain framework region 3 is backmutated. In a further embodiment, the amino acid at position 27 is backmutated from Gly (G) to Tyr (Y); the amino acid at position 28 is backmutated from Thr (T) to Glu (E); the amino acid at position 60 is backmutated from Ala (A) to Asn (N); and the amino acid at position 61 is backmutated from Gln (Q) to Gly (G). Humanized antibodies having such mutations at positions 27, 28, 60, and 61 are herein described as “H4” or “H4 (YENG)” (e.g., P1C4-H4 or P1C4-H4 (YENG)). In one embodiment of the present disclosure, the antibodies and antigen binding fragments thereof comprise framework modifications such as backmutations in the light chain. For example, in one embodiment, the antibodies comprise a mutation at position 45 and/or 47 of the light chain framework region 2. In a further embodiment, the amino acid at position 45 is mutated from Arg (R) to Lys (K) and the amino acid at position 47 is mutated from Leu (L) to Trp (W). The present disclosure also encompasses humanized antibodies that bind to CB1 and comprise framework modifications corresponding to the exemplary modifications described herein with respect to any suitable framework sequence, as well as other framework modifications that otherwise improve the properties of the antibodies. The CB1 antibodies and fragments thereof disclosed herein may be of an IgG1, IgG2, IgG3, or IgG4 isotype, or any combination thereof. The term “isotype” refers to the antibody class encoded by the heavy chain constant region genes. In addition, the heavy chain constant region may be derived from any species including, but not limited to, mouse, rat, rabbit, hamster, guinea pig, primate, llama or human. For example, in one embodiment, the CB1 antibodies and fragments thereof of the present disclosure comprise a human IgG1 Fc constant region. In another embodiment, the CB1 antibodies and fragments thereof comprise a human IgG2, human IgG4, or hybrid IgG2-IgG4 Fc constant region.

### Effector Functions and Fc Modifications

In some embodiments, present disclosure provides CB1 antibodies comprising variant Fc regions. The Fc region of an antibody is the portion of the antibody that binds to Fcγ receptors (FcγRs) and the complement molecule Clq. The Fc region plays a role in mediating antibody effector functions. “Effector functions,” as used herein in connection with antibody Fc, refers to antibody functions such as, for example, Clq binding; complement dependent cytotoxicity (CDC); Fc receptor binding; antibody-dependent cell-mediated cytotoxicity (ADCC); phagocytosis; opsonization; transcytosis; and down-regulation of cell surface receptors (e.g. B cell receptor). Such effector functions generally require the Fc region to be combined with a binding domain (e.g. an antibody variable domain) and can be assessed using various assays known in the art for evaluating such antibody effector functions. Variant Fc regions are Fc regions that comprise modifications that alter effector functions. In some embodiments, the CB1 antibodies provided herein comprise Fc region modifications that reduce, impair, or eliminate one or more effector functions. For example, in one embodiment, the antibodies and fragments thereof disclosed herein bind CB1 and exhibit reduced, impaired, or absent Clq binding and/or CDC and/or ADCC. Fc modifications may be amino acid insertions, deletions, or substitutions, or may be chemical modifications. For example, Fc region modifications may be made to increase or decrease complement binding; to increase or decrease antibody-dependent cellular cytoxicity; or to modify glycosylation. Any of the Fc modifications known in the art may be applied to the exemplary CB1 antibodies disclosed herein to alter effector function. Any of the Fc modifications known in the art may be applied to the CB1 receptor antibodies provided herein to alter effector function, antibody half life, or other antibody properties.

In one embodiment, the CB1 antibody exhibits reduced effector function. In a further embodiment, the CB1 antibody includes an IgG4 Fc region having a mutation at position 228. In a further embodiment, the amino acid at position 228 is mutated from serine(S) to proline (P) (i.e., S228P). In another embodiment, the CB1 antibody exhibits reduced effector function and includes an IgG2 Fc region having a mutation at position 330 and/or 331. In a further embodiment, the amino acid at position 330 is mutated from alanine (A) to serine(S), and/or the amino acid at position 331 is mutated from proline (P) to serine(S). In a further embodiment, the CB1 antibody includes an IgG2 Fc domain having both A330S and P331S mutations. In another embodiment, the CB1 antibody includes an IgG2/IgG4 hybrid Fc region. For example, in one embodiment, the CB1 antibody includes a CH1 and hinge region derived from IgG2, and a CH2 and CH3 region derived from IgG4.

In one embodiment, the disclosure provides an isolated nucleic acid encoding any one of the antibodies and antigen binding fragments or variants thereof disclosed herein. In some embodiments, a vector comprising the isolated nucleic acid is provided. In some embodiments, a host cell transformed with the vector is provided. In some embodiments, the host cell is a prokaryotic cell. In further embodiments, the host cell is Escherichia coli. In some embodiments, the host cell is a eukaryotic cell. In further embodiments, the eukaryotic cell is selected from the group consisting of protist cell, animal cell, plant cell and fungal cell. In some embodiments, the host cell is a mammalian cell including, but not limited to, 293, COS, NS0, and CHO and; or a fungal cell such as Saccharomyces cerevisiae; or an insect cell such as Sf9. One embodiment of the disclosure provides methods of producing the antibodies and fragments or variants described herein comprising culturing any one of the host cells also herein in a culture medium under conditions sufficient to produce the binding protein.

In some embodiments, the anti-CB1 receptor antibodies provided herein comprise the sequences provided herein or conservative variants thereof. “Conservative variants,” as used herein, include conservative amino acid substitutions, insertions, or deletions. The person of skill in the art will recognize that a conservative amino acid substitution is a substitution of one amino acid with another amino acid that has a similar structural or chemical properties, such as, for example, a similar side chain; and a conservative amino acid substitution, insertion or deletion results in a sequence that retains the biological activity of the reference sequence.

Amino acid and nucleic acid sequences of anita-CB1 antibodies useful for treatment according to the disclosure can be found at Tables 1-9, which are presented after the Examples herein. Experimental data related to the discovery, testing and validation of these antibodies is available in WO2017058771A1. Additional anti-CB1 antibodies are known and may be useful in the methods of the disclosure.

Nimacimab is in an anti-CB1 antibody that includes a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and a light chain variable region comprising a CDR1 amino acid sequence of SEQ ID NO: 355, a CDR2 amino acid sequence of SEQ ID NO: 356, and a CDR3 amino acid sequence of SEQ ID NO: 357.

In addition, nimacimab includes a heavy chain variable region comprising an amino acid sequence of SEQ ID NO: 340, and a light chain variable region comprising an amino acid sequence of SEQ ID NO:337. The antibody heavy chain amino acid sequence and light chain amino acid sequence are SEQ ID NO: 437 and SEQ ID NO:338, respectively.

In other embodiments, the anti-CB1 antibody is an antibody similar to nimacimab but including at least one mutation identified in Table 7. In addition, other antibodies useful in the methods of the disclosure include the following the following combinations of heavy and light chain variable regions:

### Combination 1

a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 694, and a CDR3 amino acid sequence of SEQ ID NO: 836.

### Combination 2

a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 835, and a CDR3 amino acid sequence of SEQ ID NO: 836.

### Combination 3

a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 694, and a CDR3 amino acid sequence of SEQ ID NO: 357.

### Combination 4

a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 834, and a CDR3 amino acid sequence of SEQ ID NO: 779.

Combinations 1, 2, 3 and 4 may include a light chain according to SEQ ID NO: 829, 830, 831 and 832, respectively and, in addition may include a heavy chain according to SEQ ID NO: 437.

Further exemplary CB1 receptor binding antibodies of the disclosure are provided below in Tables 1-9, herein, and as described in WO2017058771A1.

### EXAMPLES

The Examples that follow are illustrative of specific embodiments of the disclosure, and various uses thereof. They are set forth for explanatory purposes only and should not be construed as limiting the scope of the disclosure in any way.

### Example 1—Cohort Selection

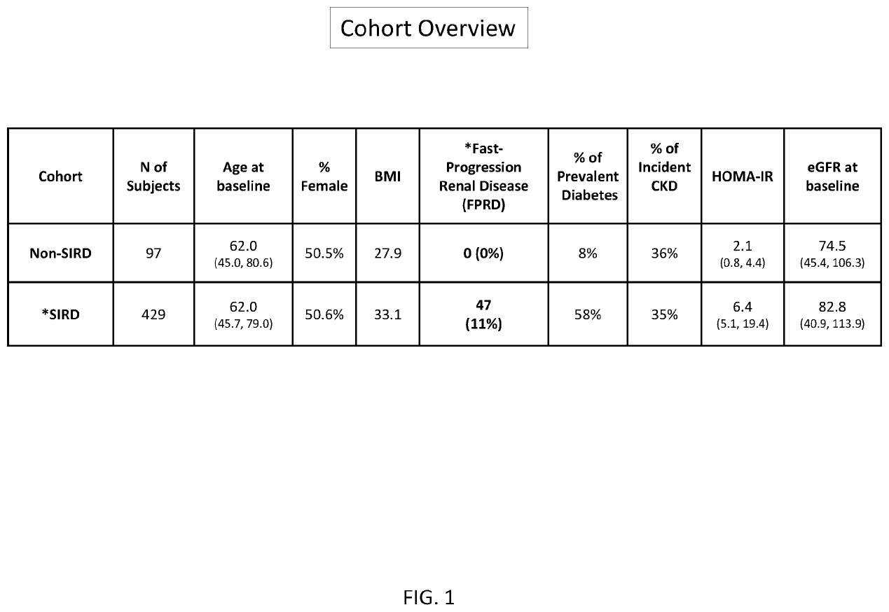

A subset of 526 plasma samples were selected from a community cohort of >5000 individuals. 429 of these samples were SIRD individuals (defined as HOMA-IR>5.0). Ninety-seven control (non-SIRD) samples were also included, as described in FIG. 1.

### Example 2—Sample Preparation

Plasma samples were prepared in 96-well plate format using an automated liquid handler. Samples underwent protein precipitation via the addition of chilled organic solvent (methanol+0.1% acetic acid) containing 6 isotopically labelled internal standards. These samples were subsequently vortexed, centrifuged at 4° C., supernatant recovered, and dried under vacuum at 40° C. Prior to analysis, samples were resuspended in methanol: water (80:20) with 0.1% acetic acid, heat-sealed, and stored at 4° C. in an autosampler awaiting imminent injection.

### Example 3—Liquid Chromatography Tandem Mass Spectroscopy

An Agilent Ultivo triple quadrupole mass spectrometer coupled to a 1290 infinity II liquid chromatography system was equipped with a Phenomenex Kinetex 1.7 um C18 100A, 100×2.1 mm column and a Waters Acquity UPLC BEH Shield RP18 1.7 um, 2.1×5 mm guard column and operated in ESI positive mode for the duration of acquisition. A linear gradient at a flow rate of 0.4 mL/min was used, consisting of water with 0.1% formic acid for mobile phase A, and acetonitrile (ACN) 0.1% formic acid for mobile phase B.

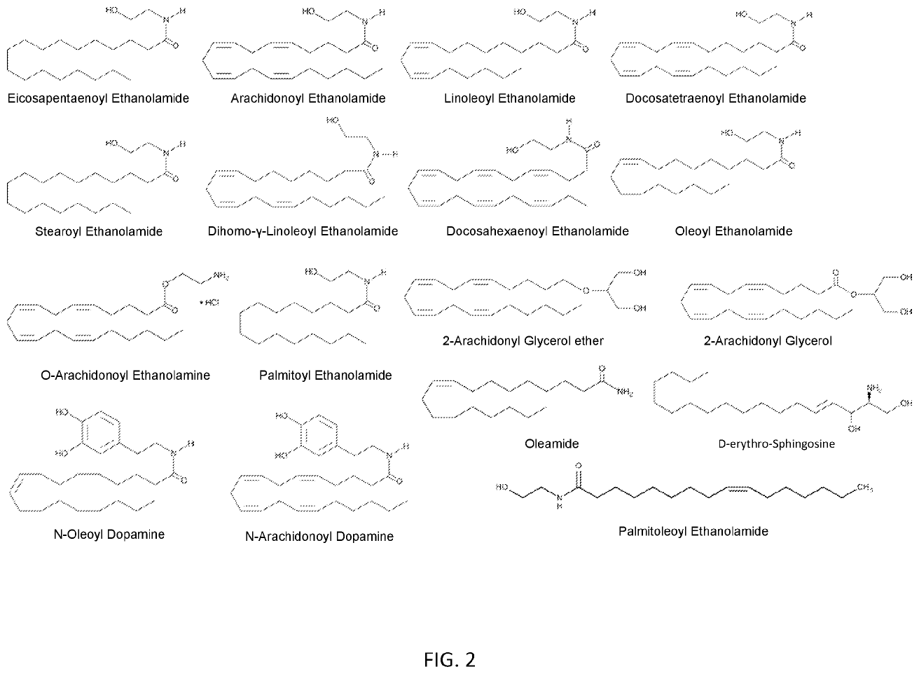

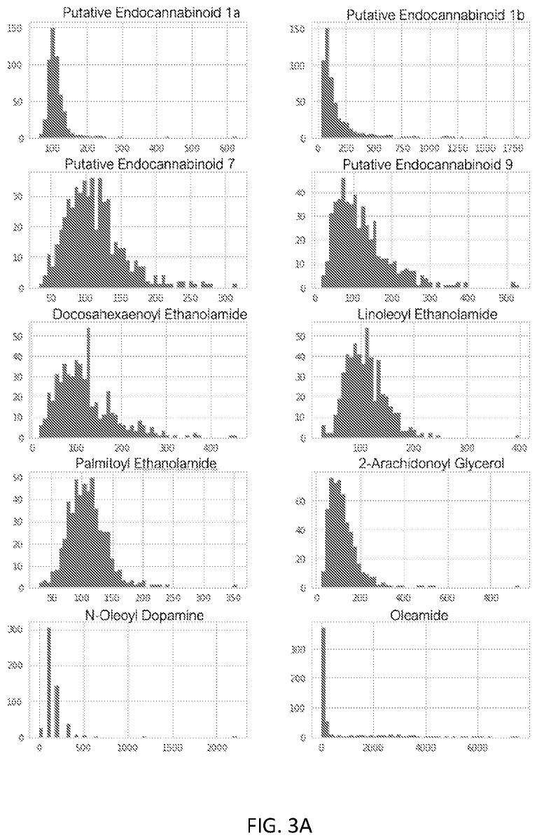

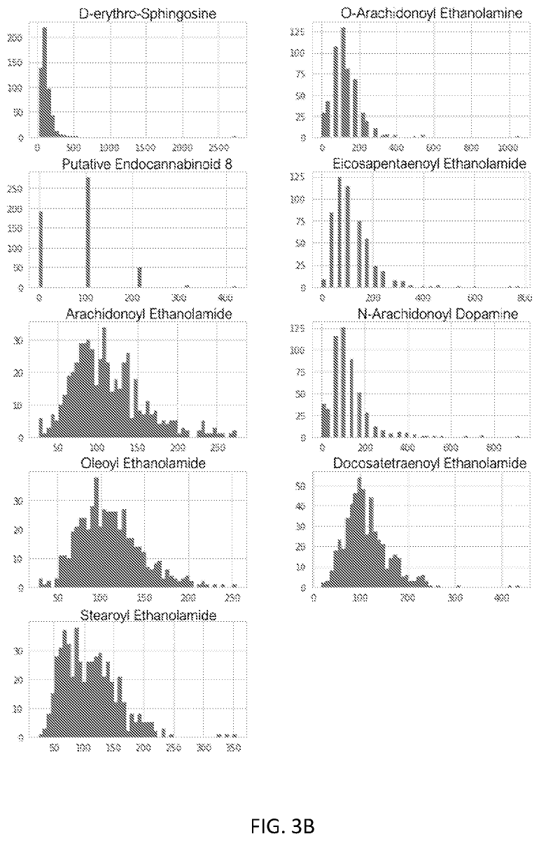

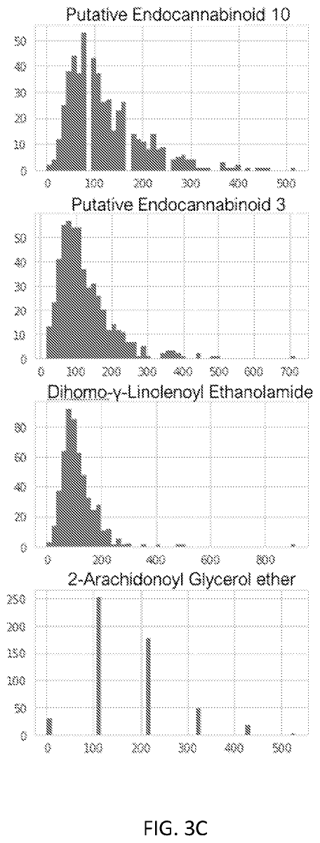

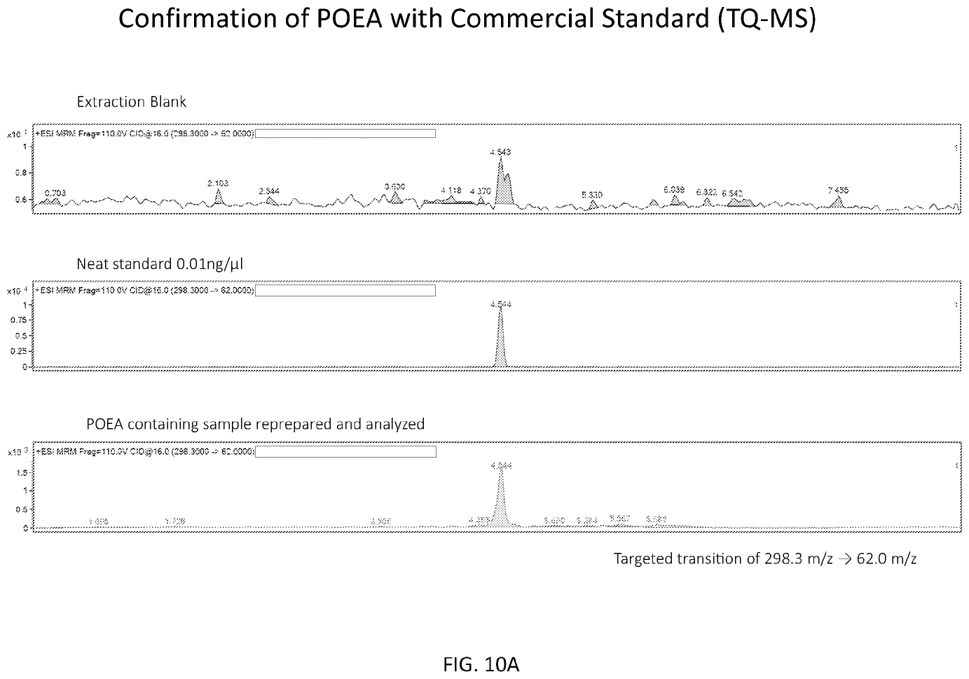

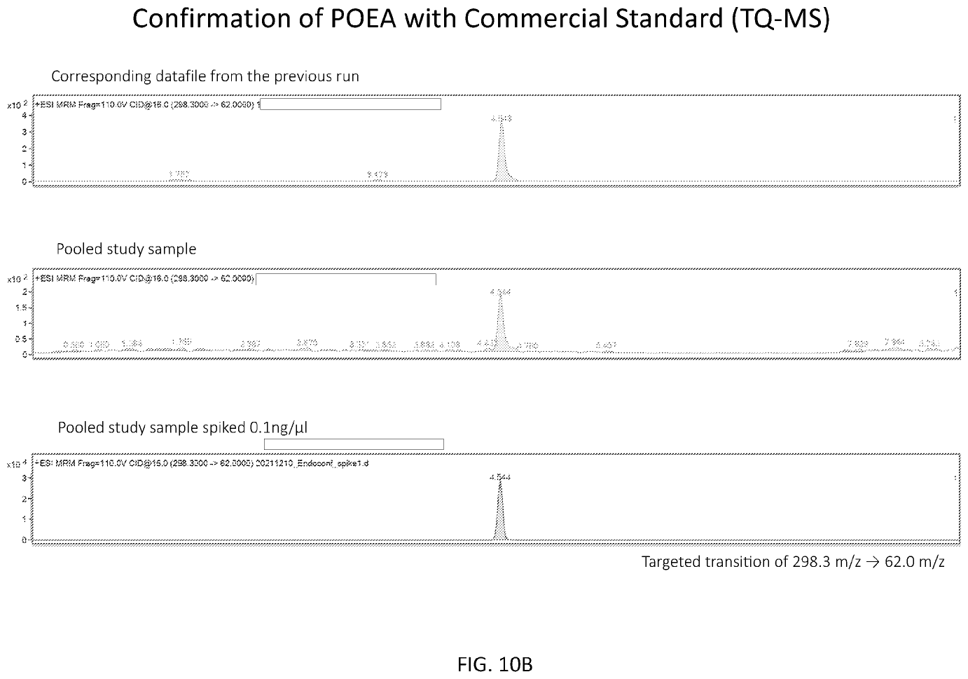

A subset of endocannabinoid structures that were measured are shown in FIG. 2, and experimental results are shown in FIG. 3A to FIG. 3C. In addition to the established group of endocannabinoids (FIG. 2, excepting pamitoleoyl ethanolamide (POEA)), several other unknown compounds were measured in the SIRD population (“putative endocannabinoids” 1a, 1b, 3, 7, 8, 9, 10; FIG. 3A to FIG. 3C). The identity of putative endocannabinoid 3 was confirmed as palmitoleoyl ethanolamide (POEA) via liquid chromatography and triple quadrupole mass spectroscopy (FIGS. 10A to 10B).

### Example 4—Statistical Analysis

For association of endocannabinoid levels with SIRD status, logistic regression was used, and confounders accounted for (including, age, sex, and BMI). Association of endocannabinoids with fast progressive renal disease, Cox Linear regression was used, and confounders (age, sex, BMI, proteinuria, HOMA-IR and GFR) accounted for. All statistical analysis was conducted in R studio.

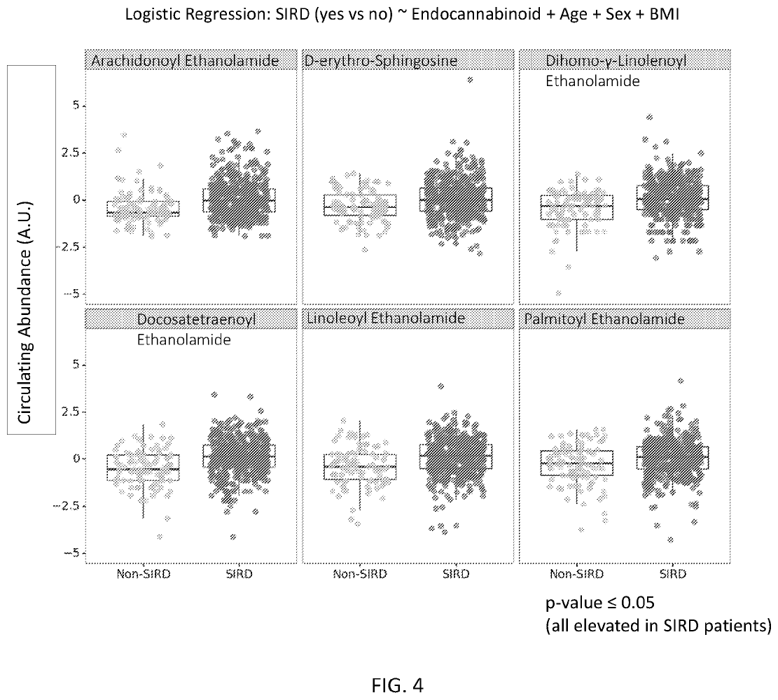

As shown in FIG. 4, six circulating endocannabinoids were significantly associated with SIRD status (adjusting for age, sex, and BMI), including arachidonoyl ethanolamide (AE), D-erythro-sphingosine (DeSph), Di-homo-γ-linolenoyl ethanolamide (DhyLE), docosatetraconoyl ethanolamide (DE), linoleoyl ethanolamide (LE), and palmitoyl ethanolamide (PE). All endocannabinoids were higher in SIRD individuals than non-SIRD controls (p<0.05), demonstrating that circulating plasma endocannabinoid levels are significantly altered in individuals with SIRD.

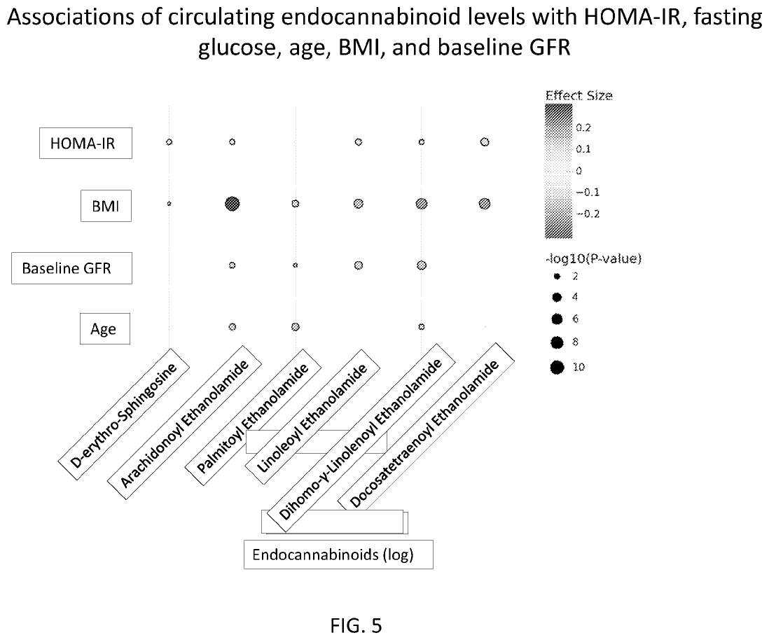

As shown in FIG. 5, of these six endocannabinoids, AE, PE, LE, DhyLE, DE showed significant association with BMI (p<0.05, effect size>0), while AE, LE and DhyLE, were associated with baseline GFR (p<0.05) though with a more modest effect size (above 0 but less than 0.2).

### Example 5—an Endocannabinoid Marker for Fast Progressive Renal Disease (FPRD)

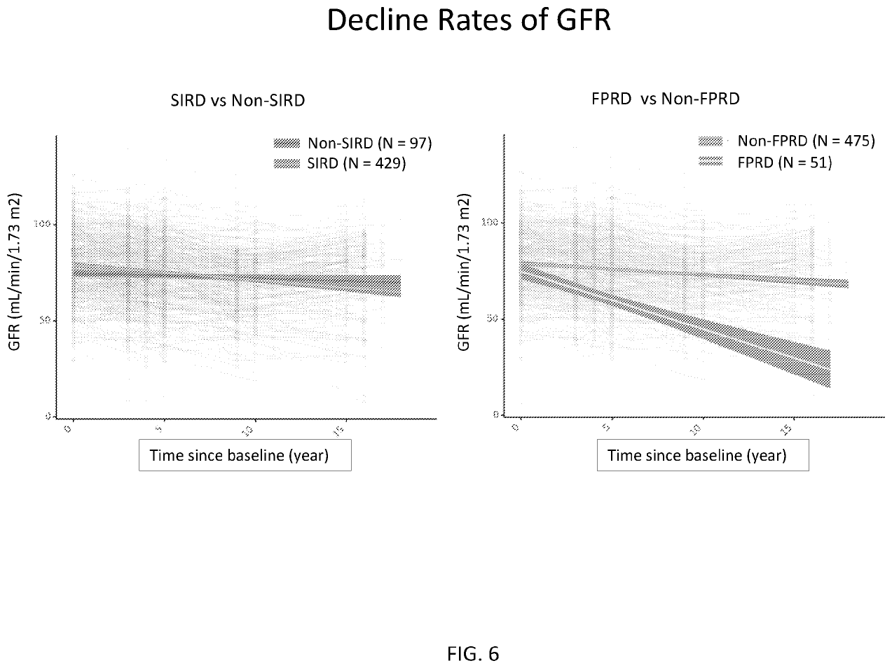

Of the 429 SIRD samples (HOMA-IR>5.0) included in the study, 11.9% of these individuals (n=51) went onto develop FPRD (defined as a GFR decline>5 units/year and end GFR<60; FIG. 6).

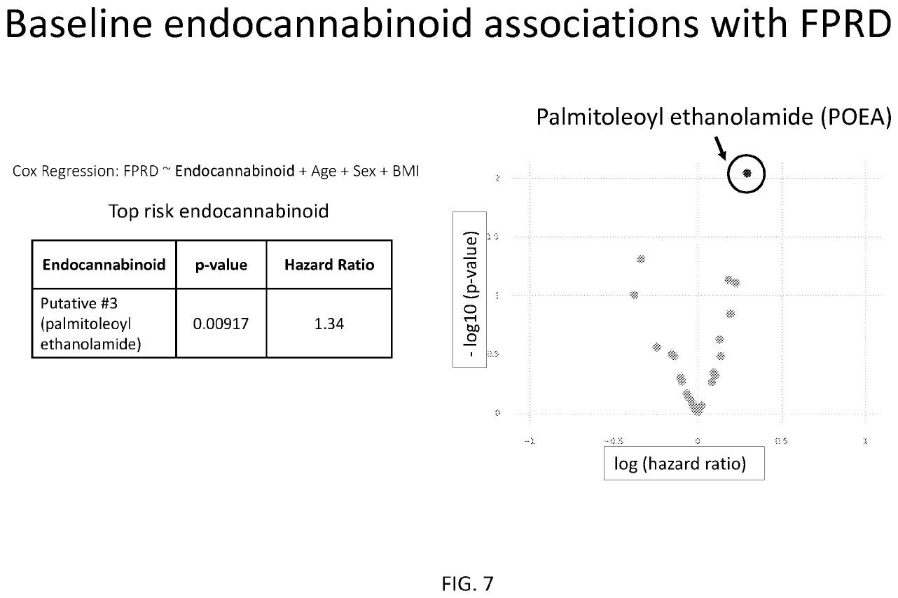

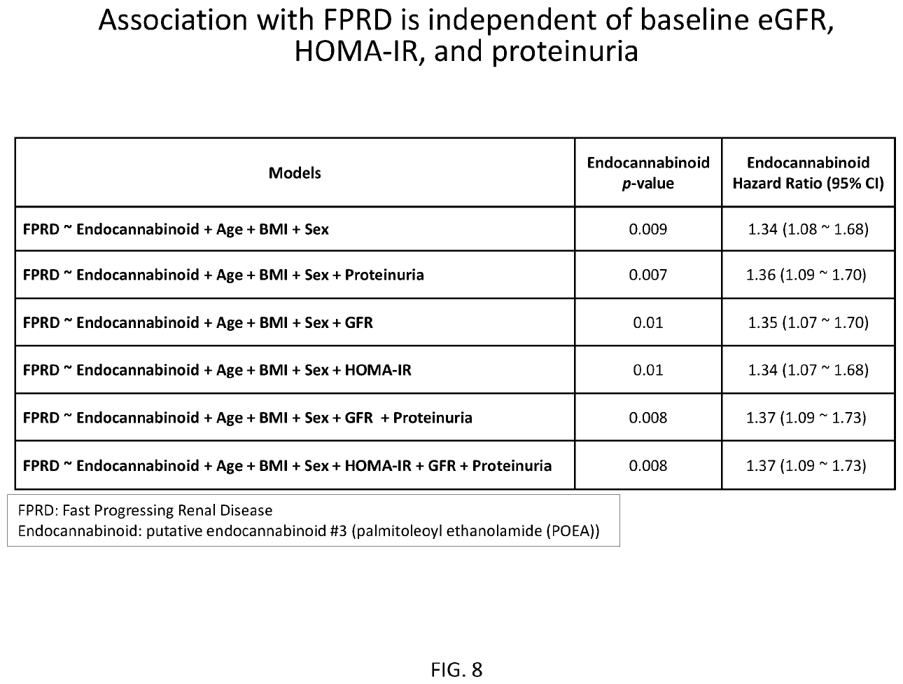

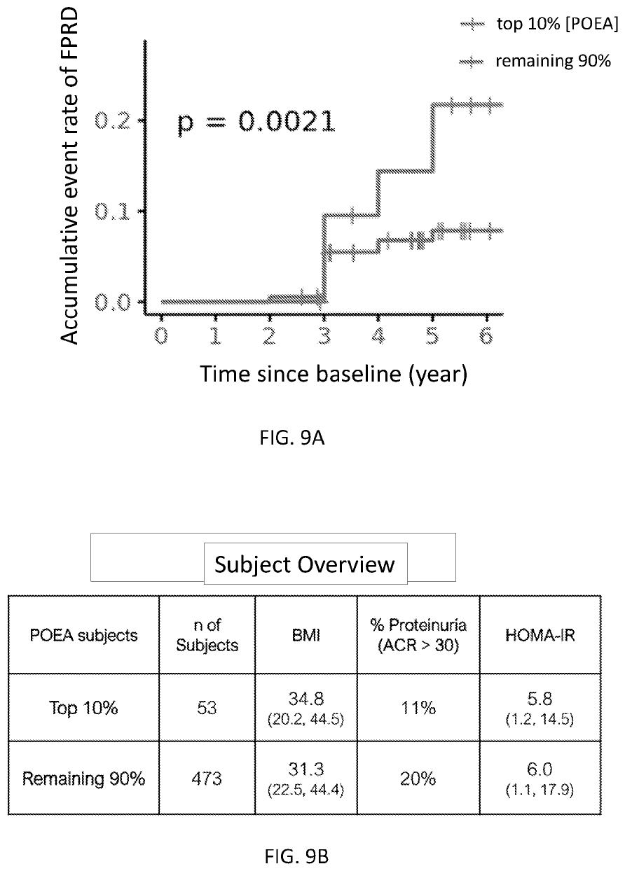

One endocannabinoid, Pamitoleoyl ethanolamide (POEA; “putative endocannabinoid 3”), was significantly associated with FPRD development (p<0.01, hazard ratio 1.34; FIG. 7, left panel), adjusted for age, sex, and BMI. POEA continued to show significant association with FPRD, independent of baseline GFR, HOMA-IR and proteinuria (p<0.01 and hazard ratio>1.3; FIG. 8). Individuals with elevated POEA at baseline had a significantly higher risk of FPRD events (p<0.01) over a six-year period as shown in the Kaplan-Meier curve of FIG. 9A, showing that in a specific sub-population of SIRD individuals that go onto develop FPRD, POEA can be a useful biomarker for predicting FPRD events and/or identifying high-risk individuals.

### Example 6—Effect of Nimacimab of POEA

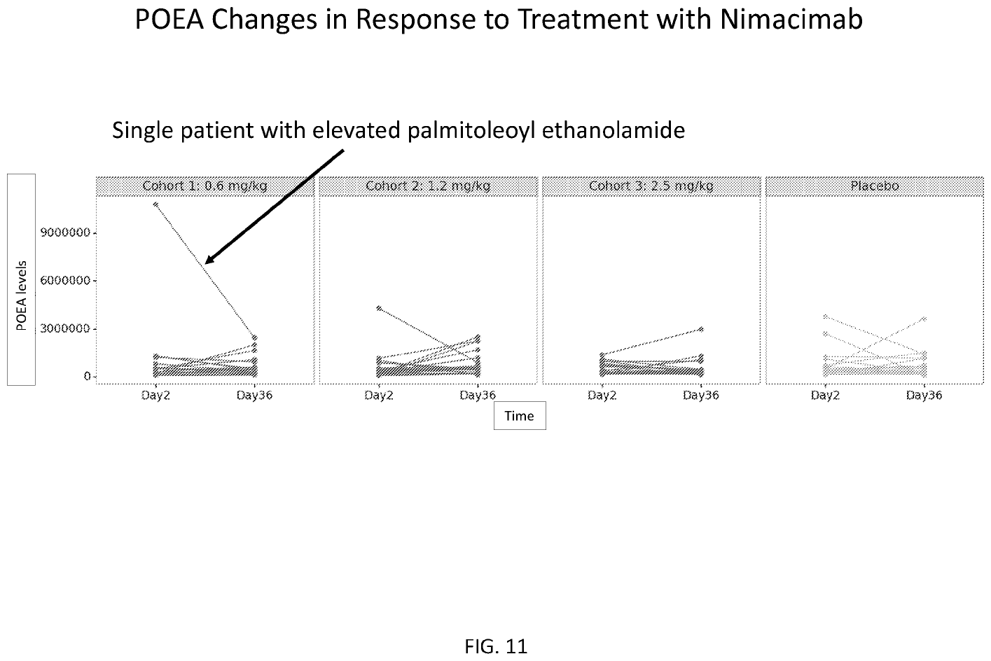

Patients received 4 weekly doses of 0.6 mg/kg, 1.2 mg/kg or 2.5 mg/kg nimacimab IV. (FIG. 11).

The clinical dose for nimacimab that showed a responder was 0.6 mg/kg. Clinical doses of 1.2 and 2.5 g/kg were also tested, but the cohorts did not capture high POEA levels in those subjects at baseline.

Having described the disclosure in detail and by reference to specific embodiments thereof, it will be apparent that modifications and variations are possible without departing from the scope of the disclosure defined in the appended claims. More specifically, although some aspects of the present disclosure are identified herein as particularly advantageous, it is contemplated that the present disclosure is not necessarily limited to these particular aspects of the disclosure.

Amino acid and nucleic acid sequences of the disclosure follow herein.

### SEQUENCES

TABLE 1
Nucleic acid and amino acid sequences of heavy chain variable regions and light
chain variable regions of exemplary CB1 receptor binding antibodies

| | | SEQ | |
|---|---|---|---|
| | Sequence | ID | |
| Name | description | NO | Sequence |
| PA13R3 - | Heavy | 1 | GAGGTCCAGCTGCAGCAGTCTGGGGCTGAGCTGGTGAGGCC |
| P1C4 | chain (HC) | | TGGGGTCTCAGTGAAGATTTCCTGCAAGGCTTCTGGCTATG |
| chimeric) | variable | | AATTCAGTTACTACTGGATGAACTGGGTGAAGCAGAGGCCT |
| | region | | GGACAGGGTCTTGAGTGGATTGGACAGATTTATCCTGGAGA |
| | nucleic | | TGGTGAAACTAAGTACAATGGAAAGTTCAAGGGTAAAGCCA |
| | acid | | CACTGACTGCAGACAAATCCTCCAACACAGCCTATATGCAG |
| | sequence | | CTCAGCAGCCTAACATCTGAGGACTCTGCGGTCTATTTCTG |
| | | | TGCAAGATCCCATGGTAACTACCTTCCTTACTGGGGCCAAG |
| | | | GGACTCTGGTCACTGTCTCTGCA |
| | HC | 2 | EVQLQQSGAELVRPGVSVKISCKASGYEFSYYWMNWVKQRP |
| | variable | | GQGLEWIGQIYPGDGETKYNGKFKGKATLTADKSSNTAYMQ |
| | region | | LSSLTSEDSAVYFCARSHGNYLPYWGQGTLVTVSA |
| | amino acid | | |
| | sequence | | |
| | HC | 3 | GCCTCCACCAAGGGCCCATCGGTCTTCCCCCTGGCACCCTC |
| | constant | | CTCCAAGAGCACCTCTGGGGGCACAGCGGCCCTGGGCTGCC |
| | region | | TGGTCAAGGACTACTTCCCCGAACCGGTGACGGTGTCATGG |
| | nucleic | | AACTCAGGCGCCCTGACCAGCGGCGTGCACACCTTCCCGGC |
| | acid | | TGTCCTACAGTCCTCAGGACTCTACTCCCTCAGCAGCGTGG |
| | sequence | | TGACCGTGCCCTCCAGCAGCTTGGGCACCCAGACCTACATC |
| | | | TGCAACGTGAATCACAAGCCCAGCAACACCAAGGTGGACAA |
| | | | GAAAGTTGAGCCCAAATCTTGTGACAAAACTCACACATGCC |
| | | | CACCGTGCCCAGCACCTGAACTCCTGGGGGGACCGTCAGTC |
| | | | TTCCTCTTCCCCCCAAAACCCAAGGACACCCTCATGATCTC |
| | | | CCGGACCCCTGAGGTCACATGCGTGGTGGTGGACGTGAGCC |
| | | | ACGAAGACCCTGAGGTCAAGTTCAACTGGTACGTGGACGGC |
| | | | GTGGAGGTGCATAATGCCAAGACAAAGCCGCGGGAGGAGCA |
| | | | GTACAACAGCACGTACCGTGTGGTCAGCGTCCTCACCGTCC |
| | | | TGCACCAGGACTGGCTGAATGGCAAGGAGTACAAGTGCAAG |
| | | | GTCTCCAACAAAGCCCTCCCAGCCCCCATCGAGAAAACCAT |
| | | | CTCCAAAGCCAAAGGGCAGCCCCGAGAACCACAGGTGTACA |
| | | | CCCTGCCCCCATCCCGGGATGAGCTGACCAAGAACCAGGTC |
| | | | AGCCTGACCTGCCTGGTCAAAGGCTTCTATCCCAGCGACAT |
| | | | CGCCGTGGAGTGGGAGAGCAATGGGCAGCCGGAGAACAACT |
| | | | ACAAGACCACGCCTCCCGTGCTGGACTCCGACGGCTCCTTC |
| | | | TTCCTCTACAGCAAGCTCACCGTGGACAAGAGCAGGTGGCA |
| | | | GCAGGGGAACGTCTTCTCATGCTCCGTGATGCATGAGGCTC |
| | | | TGCACAACCACTACACGCAGAAGAGCCTCTCCCTGTCTCCG |
| | | | GGTAAATGA |
| | HC | 4 | ASTKGPSVFPLAPSSKSTSGGTAALGCLVKDYFPEPVTVSW |
| | constant | | NSGALTSGVHTFPAVLQSSGLYSLSSVVTVPSSSLGTQTYI |
| | region | | CNVNHKPSNTKVDKKVEPKSCDKTHTCPPCPAPELLGGPSV |
| | amino acid | | FLFPPKPKDTLMISRTPEVTCVVVDVSHEDPEVKFNWYVDG |
| | sequence | | VEVHNAKTKPREEQYNSTYRVVSVLTVLHQDWLNGKEYKCK |
| | | | VSNKALPAPIEKTISKAKGQPREPQVYTLPPSRDELTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| | Light | 5 | GATATTGTTCTCACCCAGTCTCCAGCAATCATGTCTGCATC |
| | chain (LC) | | TCTAGGGGAACGGGTCACCATGACCTGCACTGCCAGCTCAA |
| | variable | | GTGTAAGTTCCAGTTACTTGCACTGGTACCAGCAGAAGCCA |
| | region | | GGATCCTCCCCCAAACTCTGGATTTATAGCACATCCAACCT |
| | nucleic | | GGCTTCTGGAGTCCCAGCTCGCTTCAGTGGCAGTGGGTCTG |
| | acid | | GGACCTCTTACTCTCTCACAATCAGCAGCATGGAGGCTGAA |
| | sequence | | GATGCTGCCACTTATTACTGCCACCAGTATCATCGTTCCCC |
| | | | ACCCACGTTCGGTGCTGGGACCAAGCTGGAGCTGAAA |
| | LC | 6 | DIVLTQSPAIMSASLGERVTMTCTASSSVSSSYLHWYQQKP |
| | variable | | GSSPKLWIYSTSNLASGVPARFSGSGSGTSYSLTISSMEAE |
| | region | | DAATYYCHQYHRSPPTFGAGTKLELK |
| | amino acid | | |
| | sequence | | |
| | LC | 7 | CGAACTGTGGCTGCACCATCTGTCTTCATCTTCCCGCCATC |
| | constant | | TGATGAGCAGTTGAAATCTGGAACTGCCTCTGTTGTGTGCC |
| | region | | TGCTGAATAACTTCTATCCCAGAGAGGCCAAAGTACAGTGG |
| | nucleic | | AAGGTGGATAACGCCCTCCAATCGGGTAACTCCCAGGAGAG |
| | acid | | TGTCACAGAGCAGGACAGCAAGGACAGCACCTACAGCCTCA |
| | sequence | | GCAGCACCCTGACGCTGAGCAAAGCAGACTACGAGAAACAC |
| | | | AAAGTCTACGCCTGCGAAGTCACCCATCAGGGCCTGAGCTT |
| | | | GCCCGTCACAAAGAGCTTCAACAGGGGAGAGTGTTAA |
| | LC | 8 | RTVAAPSVFIFPPSDEQLKSGTASVVCLLNNFYPREAKVQW |
| | constant | | KVDNALQSGNSQESVTEQDSKDSTYSLSSTLTLSKADYEKH |
| | region | | KVYACEVTHQGLSLPVTKSFNRGEC |
| | amino acid | | |
| | sequence | | |
| 36E12B6C2 | Heavy | 9 | CAGGTTCAGCTGCAGCAGTCTGGGGCTGAGCTGGTGAGGCC |
| (chimeric) | chain (HC) | | TGGGTCCTCAGTGAAGATTTCCTGCAAGGCTTCTGGCTATG |
| | variable | | AATTCAGTTACTACTGGATGAACTGGGTGAAGCAGAGGCCT |
| | region | | GGACAGGGTCTTCAGTGGATTGGACAGATTTATCCTGGAGA |
| | nucleic | | TGGTGATACTAACTACAATGGAAAGTTCAAGGGTAAAGCCA |
| | acid | | CACTGACTGCAGACAAATCCTCCAGCACAGCCTACATGCAC |
| | sequence | | CTCACCAGCCTAACGTCTGAGGACTCTGCGGTCTATTTCTG |
| | | | TGCAAGATCGGGGGGTAACCCCTTTGCTTTCTGGGGCCAAG |
| | | | GGACTCTGGTCACTGTCTCTGCA |
| | HC | 10 | QVQLQQSGAELVRPGSSVKISCKASGYEFSYYWMNWVKQRP |
| | variable | | GQGLQWIGQIYPGDGDTNYNGKFKGKATLTADKSSSTAYMH |
| | region | | LTSLTSEDSAVYFCARSGGNPFAFWGQGTLVTVSA |
| | amino acid | | |
| | sequence | | |
| | HC | 11 | GCCTCCACCAAGGGCCCATCGGTCTTCCCCCTGGCACCCTC |
| | constant | | CTCCAAGAGCACCTCTGGGGGCACAGCGGCCCTGGGCTGCC |
| | region | | TGGTCAAGGACTACTTCCCCGAACCGGTGACGGTGTCATGG |
| | nucleic | | AACTCAGGCGCCCTGACCAGCGGCGTGCACACCTTCCCGGC |
| | acid | | TGTCCTACAGTCCTCAGGACTCTACTCCCTCAGCAGCGTGG |
| | sequence | | TGACCGTGCCCTCCAGCAGCTTGGGCACCCAGACCTACATC |
| | | | TGCAACGTGAATCACAAGCCCAGCAACACCAAGGTGGACAA |
| | | | GAAAGTTGAGCCCAAATCTTGTGACAAAACTCACACATGCC |
| | | | CACCGTGCCCAGCACCTGAACTCCTGGGGGGACCGTCAGTC |
| | | | TTCCTCTTCCCCCCAAAACCCAAGGACACCCTCATGATCTC |
| | | | CCGGACCCCTGAGGTCACATGCGTGGTGGTGGACGTGAGCC |
| | | | ACGAAGACCCTGAGGTCAAGTTCAACTGGTACGTGGACGGC |
| | | | GTGGAGGTGCATAATGCCAAGACAAAGCCGCGGGAGGAGCA |
| | | | GTACAACAGCACGTACCGTGTGGTCAGCGTCCTCACCGTCC |
| | | | TGCACCAGGACTGGCTGAATGGCAAGGAGTACAAGTGCAAG |
| | | | GTCTCCAACAAAGCCCTCCCAGCCCCCATCGAGAAAACCAT |
| | | | CTCCAAAGCCAAAGGGCAGCCCCGAGAACCACAGGTGTACA |
| | | | CCCTGCCCCCATCCCGGGATGAGCTGACCAAGAACCAGGTC |
| | | | AGCCTGACCTGCCTGGTCAAAGGCTTCTATCCCAGCGACAT |
| | | | CGCCGTGGAGTGGGAGAGCAATGGGCAGCCGGAGAACAACT |
| | | | ACAAGACCACGCCTCCCGTGCTGGACTCCGACGGCTCCTTC |
| | | | TTCCTCTACAGCAAGCTCACCGTGGACAAGAGCAGGTGGCA |
| | | | GCAGGGGAACGTCTTCTCATGCTCCGTGATGCATGAGGCTC |
| | | | TGCACAACCACTACACGCAGAAGAGCCTCTCCCTGTCTCCG |
| | | | GGTAAATGA |
| | HC | 12 | ASTKGPSVFPLAPSSKSTSGGTAALGCLVKDYFPEPVTVSW |
| | constant | | NSGALTSGVHTFPAVLQSSGLYSLSSVVTVPSSSLGTQTYI |
| | region | | CNVNHKPSNTKVDKKVEPKSCDKTHTCPPCPAPELLGGPSV |
| | amino acid | | FLFPPKPKDTLMISRTPEVTCVVVDVSHEDPEVKFNWYVDG |
| | sequence | | VEVHNAKTKPREEQYNSTYRVVSVLTVLHQDWLNGKEYKCK |
| | | | VSNKALPAPIEKTISKAKGQPREPQVYTLPPSRDELTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| | Light | 13 | GATATCCAGATGACACAGACTTCATCCTCCCTGTCTGCCTC |
| | chain (LC) | | TCTGGGAGACAGAGTCACCTTCAGTTGCAGGGCAAGTCAGG |
| | variable | | ACATTAGCAATTATTTAAACTGGTATCAGCAGAAACCAGAT |
| | region | | GGAACTGTTAAACTCCTGATCTACTACACATCAAGATTACA |
| | nucleic | | CTCAGGAGTCACATCAAGGTTCCGTGGCAGTGGGTCTGGAA |
| | acid | | CAGATTATTCTCTCACCATTAGCAACCTGGAGCAAGAAGAC |
| | sequence | | GTTGCCACTTACTTTTGCCAACAGGGTCATACGCTTCCGTG |
| | | | GTCGTTCGGTGGAGGCACCAAGCTGGAAATCAAA |
| | LC | 14 | DIQMTQTSSSLSASLGDRVTFSCRASQDISNYLNWYQQKPD |
| | variable | | GTVKLLIYYTSRLHSGVTSRFRGSGSGTDYSLTISNLEQED |
| | region | | VATYFCQQGHTLPWSFGGGTKLEIK |
| | amino acid | | |
| | sequence | | |
| | LC | 15 | CGTACGGTGGCTGCACCATCTGTCTTCATCTTCCCGCCATC |
| | constant | | TGATGAGCAGTTGAAATCTGGAACTGCCTCTGTTGTGTGCC |
| | region | | TGCTGAATAACTTCTATCCCAGAGAGGCCAAAGTACAGTGG |
| | nucleic | | AAGGTGGATAACGCCCTCCAATCGGGTAACTCCCAGGAGAG |
| | acid | | TGTCACAGAGCAGGACAGCAAGGACAGCACCTACAGCCTCA |
| | sequence | | GCAGCACCCTGACGCTGAGCAAAGCAGACTACGAGAAACAC |
| | | | AAAGTCTACGCCTGCGAAGTCACCCATCAGGGCCTGAGCTC |
| | | | GCCCGTCACAAAGAGCTTCAACAGGGGAGAGTGTTAG |
| | LC | 16 | RTVAAPSVFIFPPSDEQLKSGTASVVCLLNNFYPREAKVQW |
| | constant | | KVDNALQSGNSQESVTEQDSKDSTYSLSSTLTLSKADYEKH |
| | region | | KVYACEVTHQGLSSPVTKSFNRGEC |
| | amino acid | | |
| | sequence | | |
| 36E12B6C2 | Heavy | 17 | CAGGTTCAGCTGCAGCAGTCTGGGGCTGAGCTGGTGAGGCC |
| (mouse) | chain (HC) | | TGGGTCCTCAGTGAAGATTTCCTGCAAGGCTTCTGGCTATG |
| | variable | | AATTCAGTTACTACTGGATGAACTGGGTGAAGCAGAGGCCT |
| | region | | GGACAGGGTCTTCAGTGGATTGGACAGATTTATCCTGGAGA |
| | nucleic | | TGGTGATACTAACTACAATGGAAAGTTCAAGGGTAAAGCCA |
| | acid | | CACTGACTGCAGACAAATCCTCCAGCACAGCCTACATGCAC |
| | sequence | | CTCACCAGCCTAACGTCTGAGGACTCTGCGGTCTATTTCTG |
| | | | TGCAAGATCGGGGGGTAACCCCTTTGCTTTCTGGGGCCAAG |
| | | | GGACTCTGGTCACTGTCTCTGCA |
| | HC | 18 | QVQLQQSGAELVRPGSSVKISCKASGYEFSYYWMNWVKQRP |
| | variable | | GQGLQWIGQIYPGDGDTNYNGKFKGKATLTADKSSSTAYMH |
| | region | | LTSLTSEDSAVYFCARSGGNPFAFWGQGTLVTVSA |
| | amino acid | | |
| | sequence | | |
| | HC | 19 | GCTAAAACAACAGCCCCATCGGTCTATCCACTGGCCCCTGT |
| | constant | | GTGTGGAGATACAACTGGCTCCTCGGTGACTCTAGGATGCC |
| | region | | TGGTCAAGGGTTATTTCCCTGAGCCAGTGACCTTGACCTGG |
| | nucleic | | AACTCTGGATCCCTGTCCAGTGGTGTGCACACCTTCCCAGC |
| | acid | | TGTCCTGCAGTCTGACCTCTACACCCTCAGCAGCTCAGTGA |
| | sequence | | CTGTAACCTCGAGCACCTGGCCCAGCCAGTCCATCACCTGC |
| | | | AATGTGGCCCACCCGGCAAGCAGCACCAAGGTGGACAAGAA |
| | | | AATTGAGCCCAGAGGGCCCACAATCAAGCCCTGTCCTCCAT |
| | | | GCAAATGCCCAGCACCTAACCTCTTGGGTGGACCATCCGTC |
| | | | TTCATCTTCCCTCCAAAGATCAAGGATGTACTCATGATCTC |
| | | | CCTGAGCCCCATAGTCACATGTGTGGTGGTGGATGTGAGCG |
| | | | AGGATGACCCAGATGTCCAGATCAGCTGGTTTGTGAACAAC |
| | | | GTGGAAGTACACACAGCTCAGACACAAACCCATAGAGAGGA |
| | | | TTACAACAGTACTCTCCGGGTGGTCAGTGCCCTCCCCATCC |
| | | | AGCACCAGGACTGGATGAGTGGCAAGGAGTTCAAATGCAAG |
| | | | GTCAACAACAAAGACCTCCCAGCGCCCATCGAGAGAACCAT |
| | | | CTCAAAACCCAAAGGGTCAGTAAGAGCTCCACAGGTATATG |
| | | | TCTTGCCTCCACCAGAAGAAGAGATGACTAAGAAACAGGTC |
| | | | ACTCTGACCTGCATGGTCACAGACTTCATGCCTGAAGACAT |
| | | | TTACGTGGAGTGGACCAACAACGGGAAAACAGAGCTAAACT |
| | | | ACAAGAACACTGAACCAGTCCTGGACTCTGATGGTTCTTAC |
| | | | TTCATGTACAGCAAGCTGAGAGTGGAAAAGAAGAACTGGGT |
| | | | GGAAAGAAATAGCTACTCCTGTTCAGTGGTCCACGAGGGTC |
| | | | TGCACAATCACCACACGACTAAGAGCTTCTCCCGGACTCCG |
| | | | GGTAAATGA |
| | HC | 20 | AKTTAPSVYPLAPVCGDTTGSSVTLGCLVKGYFPEPVTLTW |
| | constant | | NSGSLSSGVHTFPAVLQSDLYTLSSSVTVTSSTWPSQSITC |
| | region | | NVAHPASSTKVDKKIEPRGPTIKPCPPCKCPAPNLLGGPSV |
| | amino acid | | FIFPPKIKDVLMISLSPIVTCVVVDVSEDDPDVQISWFVNN |
| | sequence | | VEVHTAQTQTHREDYNSTLRVVSALPIQHQDWMSGKEFKCK |
| | | | VNNKDLPAPIERTISKPKGSVRAPQVYVLPPPEEEMTKKQV |
| | | | TLTCMVTDFMPEDIYVEWTNNGKTELNYKNTEPVLDSDGSY |
| | | | FMYSKLRVEKKNWVERNSYSCSVVHEGLHNHHTTKSFSRTP |
| | | | GK |
| | Light | 21 | GATATCCAGATGACACAGACTTCATCCTCCCTGTCTGCCTC |
| | chain (LC) | | TCTGGGAGACAGAGTCACCTTCAGTTGCAGGGCAAGTCAGG |
| | variable | | ACATTAGCAATTATTTAAACTGGTATCAGCAGAAACCAGAT |
| | region | | GGAACTGTTAAACTCCTGATCTACTACACATCAAGATTACA |
| | nucleic | | CTCAGGAGTCACATCAAGGTTCCGTGGCAGTGGGTCTGGAA |
| | acid | | CAGATTATTCTCTCACCATTAGCAACCTGGAGCAAGAAGAC |
| | sequence | | GTTGCCACTTACTTTTGCCAACAGGGTCATACGCTTCCGTG |
| | | | GTCGTTCGGTGGAGGCACCAAGCTGGAAATCAAA |
| | LC | 22 | DIQMTQTSSSLSASLGDRVTFSCRASQDISNYLNWYQQKPD |
| | variable | | GTVKLLIYYTSRLHSGVTSRFRGSGSGTDYSLTISNLEQED |
| | region | | VATYFCQQGHTLPWSFGGGTKLEIK |
| | amino acid | | |
| | sequence | | |
| | LC | 23 | CGGGCAGATGCTGCACCAACTGTATCCATCTTCCCACCATC |
| | constant | | CAGTGAGCAGTTAACATCTGGAGGTGCCTCAGTCGTGTGCT |
| | region | | TCTTGAACAACTTCTACCCCAAAGACATCAATGTCAAGTGG |
| | nucleic | | AAGATTGATGGCAGTGAACGACAAAATGGCGTCCTGAACAG |
| | acid | | TTGGACTGATCAGGACAGCAAAGACAGCACCTACAGCATGA |
| | sequence | | GCAGCACCCTCACGTTGACCAAGGACGAGTATGAACGACAT |
| | | | AACAGCTATACCTGTGAGGCCACTCACAAGACATCAACTTC |
| | | | ACCCATTGTCAAGAGCTTCAACAGGAATGAGTGTTAG |
| | LC | 24 | RADAAPTVSIFPPSSEQLTSGGASVVCFLNNFYPKDINVKW |
| | constant | | KIDGSERQNGVLNSWTDQDSKDSTYSMSSTLTLTKDEYERH |
| | region | | NSYTCEATHKTSTSPIVKSFNRNEC |
| | amino acid | | |
| | sequence | | |
| PA2LR3 - | Heavy | 25 | GAGGTTCAGCTGCAGCAGTCTGGGGCTGAACTGGTGAGGCC |
| P4B5 | chain (HC) | | TGGGTCCTCAGTGAAGATTTCCTGCAAGGCTTCTGGCTATG |
| chimeric) | variable | | CATTCAGTTATTACTGGATGAACTGGGTGAAGCAGAGGCCT |
| | region | | GGACAGGGTCTTGAGTGGATTGGACAGATTTATCCTGGAGA |
| | nucleic | | TGGTGATACTAACTACAGTGGAAGGTTCAAGGGTAAAGCCA |
| | acid | | CACTGACTGCAGACAAATCCTCCAGCACAGCCTACATTCAG |
| | sequence | | CTCAGCAGCCTAACATCTGAGGACTCTGCGGTCTATTTCTG |
| | | | TGCAAGATCGCACGGTAACTATTTTCCTTACTGGGGCCAAG |
| | | | GGACTCTGGTCACTGTCTCTGCA |
| | HC | 26 | EVQLQQSGAELVRPGSSVKISCKASGYAFSYYWMNWVKQRP |
| | variable | | GQGLEWIGQIYPGDGDTNYSGRFKGKATLTADKSSSTAYIQ |
| | region | | LSSLTSEDSAVYFCARSHGNYFPYWGQGTLVTVSA |
| | amino acid | | |
| | sequence | | |
| | HC | 27 | GCCTCCACCAAGGGCCCATCGGTCTTCCCCCTGGCACCCTC |
| | constant | | CTCCAAGAGCACCTCTGGGGGCACAGCGGCCCTGGGCTGCC |
| | region | | TGGTCAAGGACTACTTCCCCGAACCGGTGACGGTGTCATGG |
| | nucleic | | AACTCAGGCGCCCTGACCAGCGGCGTGCACACCTTCCCGGC |
| | acid | | TGTCCTACAGTCCTCAGGACTCTACTCCCTCAGCAGCGTGG |
| | sequence | | TGACCGTGCCCTCCAGCAGCTTGGGCACCCAGACCTACATC |
| | | | TGCAACGTGAATCACAAGCCCAGCAACACCAAGGTGGACAA |
| | | | GAAAGTTGAGCCCAAATCTTGTGACAAAACTCACACATGCC |
| | | | CACCGTGCCCAGCACCTGAACTCCTGGGGGGACCGTCAGTC |
| | | | TTCCTCTTCCCCCCAAAACCCAAGGACACCCTCATGATCTC |
| | | | CCGGACCCCTGAGGTCACATGCGTGGTGGTGGACGTGAGCC |
| | | | ACGAAGACCCTGAGGTCAAGTTCAACTGGTACGTGGACGGC |
| | | | GTGGAGGTGCATAATGCCAAGACAAAGCCGCGGGAGGAGCA |
| | | | GTACAACAGCACGTACCGTGTGGTCAGCGTCCTCACCGTCC |
| | | | TGCACCAGGACTGGCTGAATGGCAAGGAGTACAAGTGCAAG |
| | | | GTCTCCAACAAAGCCCTCCCAGCCCCCATCGAGAAAACCAT |
| | | | CTCCAAAGCCAAAGGGCAGCCCCGAGAACCACAGGTGTACA |
| | | | CCCTGCCCCCATCCCGGGATGAGCTGACCAAGAACCAGGTC |
| | | | AGCCTGACCTGCCTGGTCAAAGGCTTCTATCCCAGCGACAT |
| | | | CGCCGTGGAGTGGGAGAGCAATGGGCAGCCGGAGAACAACT |
| | | | ACAAGACCACGCCTCCCGTGCTGGACTCCGACGGCTCCTTC |
| | | | TTCCTCTACAGCAAGCTCACCGTGGACAAGAGCAGGTGGCA |
| | | | GCAGGGGAACGTCTTCTCATGCTCCGTGATGCATGAGGCTC |
| | | | TGCACAACCACTACACGCAGAAGAGCCTCTCCCTGTCTCCG |
| | | | GGTAAATGA |
| | HC | 28 | ASTKGPSVFPLAPSSKSTSGGTAALGCLVKDYFPEPVTVSW |
| | constant | | NSGALTSGVHTFPAVLQSSGLYSLSSVVTVPSSSLGTQTYI |
| | region | | CNVNHKPSNTKVDKKVEPKSCDKTHTCPPCPAPELLGGPSV |
| | amino acid | | FLFPPKPKDTLMISRTPEVTCVVVDVSHEDPEVKFNWYVDG |
| | sequence | | VEVHNAKTKPREEQYNSTYRVVSVLTVLHQDWLNGKEYKCK |
| | | | VSNKALPAPIEKTISKAKGQPREPQVYTLPPSRDELTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| | Light | 29 | GACATTGTTCTCAACCAGTCTCCAGCAATCATGTCTGCATC |
| | chain (LC) | | TCTAGGGGAACGGGTCACCATGACCTGCACTGCCAGCTCAA |
| | variable | | GTGTAAGTTCCAGTTACTTGCACTGGTACCAGCAGAAGCCA |
| | region | | GGATCCTCCCCCAAACTCTGGATTTATAGCACATCCAACCT |
| | nucleic | | GGCTTCTGGAGTCCCAGCTCGCTTCAGTGGCAGTGGGTCTG |
| | acid | | GGACCTCTTACTCTCTCACAATCAGCAGCATGGAGGCTGAA |
| | sequence | | GATGCTGCCACTTATTACTGCCACCAGTATCATCGTTCCCC |
| | | | GCTCACGTTCGGTGCTGGGACCAAACTGGAAATAAAA |
| | LC | 30 | DIVLNQSPAIMSASLGERVTMTCTASSSVSSSYLHWYQQKP |
| | variable | | GSSPKLWIYSTSNLASGVPARFSGSGSGTSYSLTISSMEAE |
| | region | | DAATYYCHQYHRSPLTFGAGTKLEIK |
| | amino acid | | |
| | sequence | | |
| | LC | 31 | CGAACTGTGGCTGCACCATCTGTCTTCATCTTCCCGCCATC |
| | constant | | TGATGAGCAGTTGAAATCTGGAACTGCCTCTGTTGTGTGCC |
| | region | | TGCTGAATAACTTCTATCCCAGAGAGGCCAAAGTACAGTGG |
| | nucleic | | AAGGTGGATAACGCCCTCCAATCGGGTAACTCCCAGGAGAG |
| | acid | | TGTCACAGAGCAGGACAGCAAGGACAGCACCTACAGCCTCA |
| | sequence | | GCAGCACCCTGACGCTGAGCAAAGCAGACTACGAGAAACAC |
| | | | AAAGTCTACGCCTGCGAAGTCACCCATCAGGGCCTGAGCTT |
| | | | GCCCGTCACAAAGAGCTTCAACAGGGGAGAGTGTTAA |
| | LC | 32 | RTVAAPSVFIFPPSDEQLKSGTASVVCLLNNFYPREAKVQW |
| | constant | | KVDNALQSGNSQESVTEQDSKDSTYSLSSTLTLSKADYEKH |
| | region | | KVYACEVTHQGLSLPVTKSFNRGEC |
| | amino acid | | |
| | sequence | | |
| PA2LR3 - | Heavy | 33 | GAGGTTCAGCTGCAGCAGTCTGGGGCTGAGCTGGTGAGGCC |
| P2D3 | chain (HC) | | TGGGTCCTCAGTGAAGATTTCCTGCAAGGCTTCTGGCTATG |
| (chimeric) | variable | | CATTCAGTTACTACTGGATGAACTGGGTGAAGCAGAGGCCT |
| | region | | GGACAGGGTCTTGAGTGGATTGGACAGATTTATCCTGGAGA |
| | nucleic | | TGGTGATACTAACTACAATGGAAAGTTCAAGGGTAAAGCCA |
| | acid | | CACTGACTGCAGACAAATCCTCCAGTACAGCCTACATGCAG |
| | sequence | | CTCAGCAGCCTAACATCTGAGGACTCTGCGGTCTATTTCTG |
| | | | TGCAAGATCGCACGGTAGCTATTTTGCTTACTGGGGCCAAG |
| | | | GGACTCTGGTCACTGTCTCTGCA |
| | HC | 34 | EVQLQQSGAELVRPGSSVKISCKASGYAFSYYWMNWVKQRP |
| | variable | | GQGLEWIGQIYPGDGDTNYNGKFKGKATLTADKSSSTAYMQ |
| | region | | LSSLTSEDSAVYFCARSHGSYFAYWGQGTLVTVSA |
| | amino acid | | |
| | sequence | | |
| | HC | 35 | GCCTCCACCAAGGGCCCATCGGTCTTCCCCCTGGCACCCTC |
| | constant | | CTCCAAGAGCACCTCTGGGGGCACAGCGGCCCTGGGCTGCC |
| | region | | TGGTCAAGGACTACTTCCCCGAACCGGTGACGGTGTCATGG |
| | nucleic | | AACTCAGGCGCCCTGACCAGCGGCGTGCACACCTTCCCGGC |
| | acid | | TGTCCTACAGTCCTCAGGACTCTACTCCCTCAGCAGCGTGG |
| | sequence | | TGACCGTGCCCTCCAGCAGCTTGGGCACCCAGACCTACATC |
| | | | TGCAACGTGAATCACAAGCCCAGCAACACCAAGGTGGACAA |
| | | | GAAAGTTGAGCCCAAATCTTGTGACAAAACTCACACATGCC |
| | | | CACCGTGCCCAGCACCTGAACTCCTGGGGGGACCGTCAGTC |
| | | | TTCCTCTTCCCCCCAAAACCCAAGGACACCCTCATGATCTC |
| | | | CCGGACCCCTGAGGTCACATGCGTGGTGGTGGACGTGAGCC |
| | | | ACGAAGACCCTGAGGTCAAGTTCAACTGGTACGTGGACGGC |
| | | | GTGGAGGTGCATAATGCCAAGACAAAGCCGCGGGAGGAGCA |
| | | | GTACAACAGCACGTACCGTGTGGTCAGCGTCCTCACCGTCC |
| | | | TGCACCAGGACTGGCTGAATGGCAAGGAGTACAAGTGCAAG |
| | | | GTCTCCAACAAAGCCCTCCCAGCCCCCATCGAGAAAACCAT |
| | | | CTCCAAAGCCAAAGGGCAGCCCCGAGAACCACAGGTGTACA |
| | | | CCCTGCCCCCATCCCGGGATGAGCTGACCAAGAACCAGGTC |
| | | | AGCCTGACCTGCCTGGTCAAAGGCTTCTATCCCAGCGACAT |
| | | | CGCCGTGGAGTGGGAGAGCAATGGGCAGCCGGAGAACAACT |
| | | | ACAAGACCACGCCTCCCGTGCTGGACTCCGACGGCTCCTTC |
| | | | TTCCTCTACAGCAAGCTCACCGTGGACAAGAGCAGGTGGCA |
| | | | GCAGGGGAACGTCTTCTCATGCTCCGTGATGCATGAGGCTC |
| | | | TGCACAACCACTACACGCAGAAGAGCCTCTCCCTGTCTCCG |
| | | | GGTAAATGA |
| | HC | 36 | ASTKGPSVFPLAPSSKSTSGGTAALGCLVKDYFPEPVTVSW |
| | constant | | NSGALTSGVHTFPAVLQSSGLYSLSSVVTVPSSSLGTQTYI |
| | region | | CNVNHKPSNTKVDKKVEPKSCDKTHTCPPCPAPELLGGPSV |
| | amino acid | | FLFPPKPKDTLMISRTPEVTCVVVDVSHEDPEVKFNWYVDG |
| | sequence | | VEVHNAKTKPREEQYNSTYRVVSVLTVLHQDWLNGKEYKCK |
| | | | VSNKALPAPIEKTISKAKGQPREPQVYTLPPSRDELTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| | Light | 37 | GATATTGAGCTGGCCCAATCTCCAGCTTCTTTGGCTGTGTC |
| | chain (LC) | | TCTAGGGCAGAGGGCCACCATATCCTGCAGAGCCAGTGAAA |
| | variable | | GTGTTGATAGTTATGGCAATAGTTTTATGCACTGGTACCAG |
| | region | | CAGAAACCAGGACAGCCACCCAAACTCCTCATCTATCTTGC |
| | nucleic | | ATCCAACCTAGAATCTGGGGTCCCTGCCAGGTTCAGCGGCA |
| | acid | | GTGGGTCTAGGGCAGACTTCACCCTCACCATTGATCCTGTG |
| | sequence | | GAGGCTGATGATGCTGCAACCTATTACTGTCTACAATATGC |
| | | | TAGTTCTCCTCCTACGTTCGGTGCTGGGACCAAACTGGAAA |
| | | | TAAAA |
| | LC | 38 | DIELAQSPASLAVSLGQRATISCRASESVDSYGNSFMHWYQ |
| | variable | | QKPGQPPKLLIYLASNLESGVPARFSGSGSRADFTLTIDPV |
| | region | | EADDAATYYCLQYASSPPTFGAGTKLEIK |
| | amino acid | | |
| | sequence | | |
| | LC | 39 | CGAACTGTGGCTGCACCATCTGTCTTCATCTTCCCGCCATC |
| | constant | | TGATGAGCAGTTGAAATCTGGAACTGCCTCTGTTGTGTGCC |
| | region | | TGCTGAATAACTTCTATCCCAGAGAGGCCAAAGTACAGTGG |
| | nucleic | | AAGGTGGATAACGCCCTCCAATCGGGTAACTCCCAGGAGAG |
| | acid | | TGTCACAGAGCAGGACAGCAAGGACAGCACCTACAGCCTCA |
| | sequence | | GCAGCACCCTGACGCTGAGCAAAGCAGACTACGAGAAACAC |
| | | | AAAGTCTACGCCTGCGAAGTCACCCATCAGGGCCTGAGCTT |
| | | | GCCCGTCACAAAGAGCTTCAACAGGGGAGAGTGTTAA |
| | LC | 40 | RTVAAPSVFIFPPSDEQLKSGTASVVCLLNNFYPREAKVQW |
| | constant | | KVDNALQSGNSQESVTEQDSKDSTYSLSSTLTLSKADYEKH |
| | region | | KVYACEVTHQGLSLPVTKSFNRGEC |
| | amino acid | | |
| | sequence | | |
| PA2LR3 - | Heavy | 41 | GAGGTCCAGCTTCAGCAATCTGGGGCTGAGCTGGTGAGGCC |
| P4B1 | chain (HC) | | TGGGTCCTCAGTGAAGATTTCCTGCAAGGCTTCTGGCTTTG |
| chimeric) | variable | | CATTCAGTAACTACTGGATGAACTGGGTGAAGCAGAGGCCT |
| | region | | GGACAGGGTCTTGAGTGGATTGGACAGATTTATCCTGGAGA |
| | nucleic | | TGGTGATACTAACTTCAATGGAAAGTTCAAGGGTAGAGCCA |
| | acid | | TACTGACTGCAGACATATCCTCCAACACAGCCTACATGCAG |
| | sequence | | CTCAGCAGCCTAACATCTGAGGACTCTGCGGTCTATTTCTG |
| | | | TGCAAGATCGCACGGTAACTATTTTCCTTACTGGGGCCAAG |
| | | | GGACTCTGGTCACTGTCTCTGCA |
| | HC | 42 | EVQLQQSGAELVRPGSSVKISCKASGFAFSNYWMNWVKQRP |
| | variable | | GQGLEWIGQIYPGDGDTNFNGKFKGRAILTADISSNTAYMQ |
| | region | | LSSLTSEDSAVYFCARSHGNYFPYWGQGTLVTVSA |
| | amino acid | | |
| | sequence | | |
| | HC | 43 | GCCTCCACCAAGGGCCCATCGGTCTTCCCCCTGGCACCCTC |
| | constant | | CTCCAAGAGCACCTCTGGGGGCACAGCGGCCCTGGGCTGCC |
| | region | | TGGTCAAGGACTACTTCCCCGAACCGGTGACGGTGTCATGG |
| | nucleic | | AACTCAGGCGCCCTGACCAGCGGCGTGCACACCTTCCCGGC |
| | acid | | TGTCCTACAGTCCTCAGGACTCTACTCCCTCAGCAGCGTGG |
| | sequence | | TGACCGTGCCCTCCAGCAGCTTGGGCACCCAGACCTACATC |
| | | | TGCAACGTGAATCACAAGCCCAGCAACACCAAGGTGGACAA |
| | | | GAAAGTTGAGCCCAAATCTTGTGACAAAACTCACACATGCC |
| | | | CACCGTGCCCAGCACCTGAACTCCTGGGGGGACCGTCAGTC |
| | | | TTCCTCTTCCCCCCAAAACCCAAGGACACCCTCATGATCTC |
| | | | CCGGACCCCTGAGGTCACATGCGTGGTGGTGGACGTGAGCC |
| | | | ACGAAGACCCTGAGGTCAAGTTCAACTGGTACGTGGACGGC |
| | | | GTGGAGGTGCATAATGCCAAGACAAAGCCGCGGGAGGAGCA |
| | | | GTACAACAGCACGTACCGTGTGGTCAGCGTCCTCACCGTCC |
| | | | TGCACCAGGACTGGCTGAATGGCAAGGAGTACAAGTGCAAG |
| | | | GTCTCCAACAAAGCCCTCCCAGCCCCCATCGAGAAAACCAT |
| | | | CTCCAAAGCCAAAGGGCAGCCCCGAGAACCACAGGTGTACA |
| | | | CCCTGCCCCCATCCCGGGATGAGCTGACCAAGAACCAGGTC |
| | | | AGCCTGACCTGCCTGGTCAAAGGCTTCTATCCCAGCGACAT |
| | | | CGCCGTGGAGTGGGAGAGCAATGGGCAGCCGGAGAACAACT |
| | | | ACAAGACCACGCCTCCCGTGCTGGACTCCGACGGCTCCTTC |
| | | | TTCCTCTACAGCAAGCTCACCGTGGACAAGAGCAGGTGGCA |
| | | | GCAGGGGAACGTCTTCTCATGCTCCGTGATGCATGAGGCTC |
| | | | TGCACAACCACTACACGCAGAAGAGCCTCTCCCTGTCTCCG |
| | | | GGTAAATGA |
| | HC | 44 | ASTKGPSVFPLAPSSKSTSGGTAALGCLVKDYFPEPVTVSW |
| | constant | | NSGALTSGVHTFPAVLQSSGLYSLSSVVTVPSSSLGTQTYI |
| | region | | CNVNHKPSNTKVDKKVEPKSCDKTHTCPPCPAPELLGGPSV |
| | amino acid | | FLFPPKPKDTLMISRTPEVTCVVVDVSHEDPEVKFNWYVDG |
| | sequence | | VEVHNAKTKPREEQYNSTYRVVSVLTVLHQDWLNGKEYKCK |
| | | | VSNKALPAPIEKTISKAKGQPREPQVYTLPPSRDELTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| | Light | 45 | CAAATTGTGTTGACACAGTCTCCAGCAATCATGTCTGCATC |
| | chain (LC) | | TCTAGGGGAACGGGTCACCATGACCTGCACTGCCAGCTCAA |
| | variable | | GTGTAAGTTCCAGTTACTTGCACTGGTACCAGCAGAAGCCA |
| | region | | GGATCCTCCCCCAAACTCTGGATTTATAGCACATCCAACCT |
| | nucleic | | GGCTTCTGGAGTCCCAGCTCGCTTCAGTGGCAGTGGGTCTG |
| | acid | | GGACCTCTTACTCTCTCACAATCAGCAGCATGGAGGCTGAA |
| | sequence | | GATGCTGCCACTTATTACTGCCACCAGTATCATCGTTCCCC |
| | | | GCTCACGTTCGGTGCTGGGACCAAGCTGGAGCTGAAA |
| | LC | 46 | QIVLTQSPAIMSASLGERVTMTCTASSSVSSSYLHWYQQKP |
| | variable | | GSSPKLWIYSTSNLASGVPARFSGSGSGTSYSLTISSMEAE |
| | region | | DAATYYCHQYHRSPLTFGAGTKLELK |
| | amino acid | | |
| | sequence | | |
| | LC | 47 | CGAACTGTGGCTGCACCATCTGTCTTCATCTTCCCGCCATC |
| | constant | | TGATGAGCAGTTGAAATCTGGAACTGCCTCTGTTGTGTGCC |
| | region | | TGCTGAATAACTTCTATCCCAGAGAGGCCAAAGTACAGTGG |
| | nucleic | | AAGGTGGATAACGCCCTCCAATCGGGTAACTCCCAGGAGAG |
| | acid | | TGTCACAGAGCAGGACAGCAAGGACAGCACCTACAGCCTCA |
| | sequence | | GCAGCACCCTGACGCTGAGCAAAGCAGACTACGAGAAACAC |
| | | | AAAGTCTACGCCTGCGAAGTCACCCATCAGGGCCTGAGCTT |
| | | | GCCCGTCACAAAGAGCTTCAACAGGGGAGAGTGTTAA |
| | LC | 48 | RTVAAPSVFIFPPSDEQLKSGTASVVCLLNNFYPREAKVQW |
| | constant | | KVDNALQSGNSQESVTEQDSKDSTYSLSSTLTLSKADYEKH |
| | region | | KVYACEVTHQGLSLPVTKSFNRGEC |
| | amino acid | | |
| | sequence | | |
| PA2LR3 - | Heavy | 49 | GAGGTCCAGCTTCAGCAATCTGGGGCTGAGCTGGTGAGGCC |
| P6B12 | chain (HC) | | TGGGTCCTCAGTGAAGATTTCCTGCAAGGCTTCTGGCTATG |
| chimeric) | variable | | CATTCAGTTACTACTGGATGAACTGGGTGAAGCAGAGGCCT |
| | region | | GGACAGGGTCTTGAGTGGATTGGACAGATTTATCCTGGAGA |
| | nucleic | | TGGTGATACTAACTACAATGGAAAGTTCAAGGGTAAAGCCA |
| | acid | | CACTGACTGCAGACAAATCCTCCAGTACAGCCTACATGCAG |
| | sequence | | CTCAGCAGCCTAACATCTGAGGACTCTGCGGTCTATTTCTG |
| | | | TGCAAGATCGCACGGTAACTATTTTGCTTACTGGGGCCAAG |
| | | | GGACTCTGGTCACTGTCTCTGCA |
| | HC | 50 | EVQLQQSGAELVRPGSSVKISCKASGYAFSYYWMNWVKQRP |
| | variable | | GQGLEWIGQIYPGDGDTNYNGKFKGKATLTADKSSSTAYMQ |
| | region | | LSSLTSEDSAVYFCARSHGNYFAYWGQGTLVTVSA |
| | amino acid | | |
| | sequence | | |
| | HC | 51 | GCCTCCACCAAGGGCCCATCGGTCTTCCCCCTGGCACCCTC |
| | constant | | CTCCAAGAGCACCTCTGGGGGCACAGCGGCCCTGGGCTGCC |
| | region | | TGGTCAAGGACTACTTCCCCGAACCGGTGACGGTGTCATGG |
| | nucleic | | AACTCAGGCGCCCTGACCAGCGGCGTGCACACCTTCCCGGC |
| | acid | | TGTCCTACAGTCCTCAGGACTCTACTCCCTCAGCAGCGTGG |
| | sequence | | TGACCGTGCCCTCCAGCAGCTTGGGCACCCAGACCTACATC |
| | | | TGCAACGTGAATCACAAGCCCAGCAACACCAAGGTGGACAA |
| | | | GAAAGTTGAGCCCAAATCTTGTGACAAAACTCACACATGCC |
| | | | CACCGTGCCCAGCACCTGAACTCCTGGGGGGACCGTCAGTC |
| | | | TTCCTCTTCCCCCCAAAACCCAAGGACACCCTCATGATCTC |
| | | | CCGGACCCCTGAGGTCACATGCGTGGTGGTGGACGTGAGCC |
| | | | ACGAAGACCCTGAGGTCAAGTTCAACTGGTACGTGGACGGC |
| | | | GTGGAGGTGCATAATGCCAAGACAAAGCCGCGGGAGGAGCA |
| | | | GTACAACAGCACGTACCGTGTGGTCAGCGTCCTCACCGTCC |
| | | | TGCACCAGGACTGGCTGAATGGCAAGGAGTACAAGTGCAAG |
| | | | GTCTCCAACAAAGCCCTCCCAGCCCCCATCGAGAAAACCAT |
| | | | CTCCAAAGCCAAAGGGCAGCCCCGAGAACCACAGGTGTACA |
| | | | CCCTGCCCCCATCCCGGGATGAGCTGACCAAGAACCAGGTC |
| | | | AGCCTGACCTGCCTGGTCAAAGGCTTCTATCCCAGCGACAT |
| | | | CGCCGTGGAGTGGGAGAGCAATGGGCAGCCGGAGAACAACT |
| | | | ACAAGACCACGCCTCCCGTGCTGGACTCCGACGGCTCCTTC |
| | | | TTCCTCTACAGCAAGCTCACCGTGGACAAGAGCAGGTGGCA |
| | | | GCAGGGGAACGTCTTCTCATGCTCCGTGATGCATGAGGCTC |
| | | | TGCACAACCACTACACGCAGAAGAGCCTCTCCCTGTCTCCG |
| | | | GGTAAATGA |
| | HC | 52 | ASTKGPSVFPLAPSSKSTSGGTAALGCLVKDYFPEPVTVSW |
| | constant | | NSGALTSGVHTFPAVLQSSGLYSLSSVVTVPSSSLGTQTYI |
| | region | | CNVNHKPSNTKVDKKVEPKSCDKTHTCPPCPAPELLGGPSV |
| | amino acid | | FLFPPKPKDTLMISRTPEVTCVVVDVSHEDPEVKFNWYVDG |
| | sequence | | VEVHNAKTKPREEQYNSTYRVVSVLTVLHQDWLNGKEYKCK |
| | | | VSNKALPAPIEKTISKAKGQPREPQVYTLPPSRDELTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| | Light | 53 | CAAATTGTACTCACCCAGTCTCCAGCAATCATGTCTGCATC |
| | chain (LC) | | TCTAGGGGAACGGGTCACCATGACCTGCACTGCCAGCTCAA |
| | variable | | GTGTAAGTTCCAGTTACTTGCACTGGTACCAGCAGAAGCCA |
| | region | | GGATCCTCCCCCAAACTCTGGATTTATAGCACATCCAACCT |
| | nucleic | | GGCTTCTGGAGTCCCAGCTCGCTTCAGTGGCAGTGGGTCTG |
| | acid | | GGACCTCTTACTCTCTCACAATCAGCAGCATGGAGGCTGAA |
| | sequence | | GATGCTGCCACTTATTACTGCCACCAGTATCATCGTTCCCC |
| | | | CCTCGCGTTCGGTGCTGGGACCAAGCTGGAGCTGAAA |
| | LC | 54 | QIVLTQSPAIMSASLGERVTMTCTASSSVSSSYLHWYQQKP |
| | variable | | GSSPKLWIYSTSNLASGVPARFSGSGSGTSYSLTISSMEAE |
| | region | | DAATYYCHQYHRSPLAFGAGTKLELK |
| | amino acid | | |
| | sequence | | |
| | LC | 55 | CGAACTGTGGCTGCACCATCTGTCTTCATCTTCCCGCCATC |
| | constant | | TGATGAGCAGTTGAAATCTGGAACTGCCTCTGTTGTGTGCC |
| | region | | TGCTGAATAACTTCTATCCCAGAGAGGCCAAAGTACAGTGG |
| | nucleic | | AAGGTGGATAACGCCCTCCAATCGGGTAACTCCCAGGAGAG |
| | acid | | TGTCACAGAGCAGGACAGCAAGGACAGCACCTACAGCCTCA |
| | sequence | | GCAGCACCCTGACGCTGAGCAAAGCAGACTACGAGAAACAC |
| | | | AAAGTCTACGCCTGCGAAGTCACCCATCAGGGCCTGAGCTT |
| | | | GCCCGTCACAAAGAGCTTCAACAGGGGAGAGTGTTAA |
| | LC | 56 | RTVAAPSVFIFPPSDEQLKSGTASVVCLLNNFYPREAKVQW |
| | constant | | KVDNALQSGNSQESVTEQDSKDSTYSLSSTLTLSKADYEKH |
| | region | | KVYACEVTHQGLSLPVTKSFNRGEC |
| | amino acid | | |
| | sequence | | |
| PA2LR3 - | Heavy | 57 | GAGGTTCAGCTTCAGCAATCTGGGGCTGAGCTGGTGAGGCC |
| P6G7 | chain (HC) | | TGGGTCCTCAGTGAAGATTTCCTGCAAGGCTTCTGGCTTTG |
| (chimeric) | variable | | CATTCAGTAACTACTGGATGAACTGGGTGAAGCAGAGGCCC |
| | region | | GGACAGGGTCTTGAGTGGATTGGACAGATTTATCCTGGAGA |
| | nucleic | | TGGTGATACTAACTTCAATGGAAAGTTCAAGGGTAGAGCCA |
| | acid | | TACTGACTGCAGACATATCCTCCAACACAGCCTACATGCAG |
| | sequence | | CTCAGCAGCCTAACATCTGAGGACTCTGCGGTCTATTTCTG |
| | | | TGCAAGATCGCACGGTAACTATTTTCCTTACTGGGGCCAAG |
| | | | GGACTCTGGTCACTGTCTCTGCA |
| | HC | 58 | EVQLQQSGAELVRPGSSVKISCKASGFAFSNYWMNWVKQRP |
| | variable | | GQGLEWIGQIYPGDGDTNFNGKFKGRAILTADISSNTAYMQ |
| | region | | LSSLTSEDSAVYFCARSHGNYFPYWGQGTLVTVSA |
| | amino acid | | |
| | sequence | | |
| | HC | 59 | GCCTCCACCAAGGGCCCATCGGTCTTCCCCCTGGCACCCTC |
| | constant | | CTCCAAGAGCACCTCTGGGGGCACAGCGGCCCTGGGCTGCC |
| | region | | TGGTCAAGGACTACTTCCCCGAACCGGTGACGGTGTCATGG |
| | nucleic | | AACTCAGGCGCCCTGACCAGCGGCGTGCACACCTTCCCGGC |
| | acid | | TGTCCTACAGTCCTCAGGACTCTACTCCCTCAGCAGCGTGG |
| | sequence | | TGACCGTGCCCTCCAGCAGCTTGGGCACCCAGACCTACATC |
| | | | TGCAACGTGAATCACAAGCCCAGCAACACCAAGGTGGACAA |
| | | | GAAAGTTGAGCCCAAATCTTGTGACAAAACTCACACATGCC |
| | | | CACCGTGCCCAGCACCTGAACTCCTGGGGGGACCGTCAGTC |
| | | | TTCCTCTTCCCCCCAAAACCCAAGGACACCCTCATGATCTC |
| | | | CCGGACCCCTGAGGTCACATGCGTGGTGGTGGACGTGAGCC |
| | | | ACGAAGACCCTGAGGTCAAGTTCAACTGGTACGTGGACGGC |
| | | | GTGGAGGTGCATAATGCCAAGACAAAGCCGCGGGAGGAGCA |
| | | | GTACAACAGCACGTACCGTGTGGTCAGCGTCCTCACCGTCC |
| | | | TGCACCAGGACTGGCTGAATGGCAAGGAGTACAAGTGCAAG |
| | | | GTCTCCAACAAAGCCCTCCCAGCCCCCATCGAGAAAACCAT |
| | | | CTCCAAAGCCAAAGGGCAGCCCCGAGAACCACAGGTGTACA |
| | | | CCCTGCCCCCATCCCGGGATGAGCTGACCAAGAACCAGGTC |
| | | | AGCCTGACCTGCCTGGTCAAAGGCTTCTATCCCAGCGACAT |
| | | | CGCCGTGGAGTGGGAGAGCAATGGGCAGCCGGAGAACAACT |
| | | | ACAAGACCACGCCTCCCGTGCTGGACTCCGACGGCTCCTTC |
| | | | TTCCTCTACAGCAAGCTCACCGTGGACAAGAGCAGGTGGCA |
| | | | GCAGGGGAACGTCTTCTCATGCTCCGTGATGCATGAGGCTC |
| | | | TGCACAACCACTACACGCAGAAGAGCCTCTCCCTGTCTCCG |
| | | | GGTAAATGA |
| | HC | 60 | ASTKGPSVFPLAPSSKSTSGGTAALGCLVKDYFPEPVTVSW |
| | constant | | NSGALTSGVHTFPAVLQSSGLYSLSSVVTVPSSSLGTQTYI |
| | region | | CNVNHKPSNTKVDKKVEPKSCDKTHTCPPCPAPELLGGPSV |
| | amino acid | | FLFPPKPKDTLMISRTPEVTCVVVDVSHEDPEVKFNWYVDG |
| | sequence | | VEVHNAKTKPREEQYNSTYRVVSVLTVLHQDWLNGKEYKCK |
| | | | VSNKALPAPIEKTISKAKGQPREPQVYTLPPSRDELTKNQV |
| | | | SLTCLVKGFYPSDIAVEWESNGQPENNYKTTPPVLDSDGSF |
| | | | FLYSKLTVDKSRWQQGNVFSCSVMHEALHNHYTQKSLSLSP |
| | | | GK |
| | Light | 61 | GATATTGTGCTAACTCAGTCTCCAGCAATCATGTCCGCATC |
| | chain (LC) | | TCTAGGGGAACGGGTCACCATGACCTGCACTGCCAGCTCAA |
| | variable | | GTGTAAGTTCCAGTTACTTACACTGGTACCAGCAGAAGCCA |
| | region | | GGATCCTCCCCCAAACTCTGGATTTATAGCACCTCCAACCT |
| | nucleic | | GGCTTCTGGAGTCCCAGCTCGCTTCAGTGGCAGTGGGTCTG |
| | acid | | GGACCTCTTACTCTCTCACAATCAGCAGCATGGAGGCTGAA |
| | sequence | | GATGCTGCCACTTATTACTGCCACCAGCATCATCGTTCCCC |
| | | | ACCCACGTTCGGTGCTGGGACCAAGCTGGAGCTGAAA |
| | LC | 62 | DIVLTQSPAIMSASLGERVTMTCTASSSVSSSYLHWYQQKP |
| | variable | | GSSPKLWIYSTSNLASGVPARFSGSGSGTSYSLTISSMEAE |
| | region | | DAATYYCHQHHRSPPTFGAGTKLELK |
| | amino acid | | |
| | sequence | | |
| | LC | 63 | CGAACTGTGGCTGCACCATCTGTCTTCATCTTCCCGCCATC |
| | constant | | TGATGAGCAGTTGAAATCTGGAACTGCCTCTGTTGTGTGCC |
| | region | | TGCTGAATAACTTCTATCCCAGAGAGGCCAAAGTACAGTGG |
| | nucleic | | AAGGTGGATAACGCCCTCCAATCGGGTAACTCCCAGGAGAG |
| | acid | | TGTCACAGAGCAGGACAGCAAGGACAGCACCTACAGCCTCA |
| | sequence | | GCAGCACCCTGACGCTGAGCAAAGCAGACTACGAGAAACAC |
| | | | AAAGTCTACGCCTGCGAAGTCACCCATCAGGGCCTGAGCTT |
| | | | GCCCGTCACAAAGAGCTTCAACAGGGGAGAGTGTTAA |
| | LC | 64 | RTVAAPSVFIFPPSDEQLKSGTASVVCLLNNFYPREAKVQW |
| | constant | | KVDNALQSGNSQESVTEQDSKDSTYSLSSTLTLSKADYEKH |
| | region | | KVYACEVTHQGLSLPVTKSFNRGEC |
| | amino acid | | |
| | sequence | | |

TABLE 2
SEQ ID NOs for additional CB1 receptor binding antibodies

| | | SEQ ID |
|---|---|---|
| Name | Sequence description | NO |
| PA2LR3-P1G6 (chimeric) | Heavy chain (HC) variable nucleic acid | 65 |
| | HC variable region amino acid | 66 |
| | HC constant region nucleic acid | 67 |
| | HC constant region amino acid | 68 |
| | Light chain (LC) variable region nucleic acid | 69 |
| | LC variable region amino acid | 70 |
| | LC constant region nucleic acid | 71 |
| | LC constant region amino acid | 72 |
| PA2LR3-P1H4 (chimeric) | Heavy chain (HC) variable nucleic acid | 73 |
| | HC variable region amino acid | 74 |
| | HC constant region nucleic acid | 75 |
| | HC constant region amino acid | 76 |
| | Light chain (LC) variable region nucleic acid | 77 |
| | LC variable region amino acid | 78 |
| | LC constant region nucleic acid | 79 |
| | LC constant region amino acid | 80 |
| PA2LR3-P2B8 (chimeric) | Heavy chain (HC) variable nucleic acid | 81 |
| | HC variable region amino acid | 82 |
| | HC constant region nucleic acid | 83 |
| | HC constant region amino acid | 84 |
| | Light chain (LC) variable region nucleic acid | 85 |
| | LC variable region amino acid | 86 |
| | LC constant region nucleic acid | 87 |
| | LC constant region amino acid | 88 |
| PA2LR3-P2E5 (chimeric) | Heavy chain (HC) variable nucleic acid | 89 |
| | HC variable region amino acid | 90 |
| | HC constant region nucleic acid | 91 |
| | HC constant region amino acid | 92 |
| | Light chain (LC) variable region nucleic acid | 93 |
| | LC variable region amino acid | 94 |
| | LC constant region nucleic acid | 95 |
| | LC constant region amino acid | 96 |
| PA2LR3-P3A8 (chimeric) | Heavy chain (HC) variable nucleic acid | 97 |
| | HC variable region amino acid | 98 |
| | HC constant region nucleic acid | 99 |
| | HC constant region amino acid | 100 |
| | Light chain (LC) variable region nucleic acid | 101 |
| | LC variable region amino acid | 102 |
| | LC constant region nucleic acid | 103 |
| | LC constant region amino acid | 104 |
| PA2LR3-P3B10 (chimeric) | Heavy chain (HC) variable nucleic acid | 105 |
| | HC variable region amino acid | 106 |
| | HC constant region nucleic acid | 107 |
| | HC constant region amino acid | 108 |
| | Light chain (LC) variable region nucleic acid | 109 |
| | LC variable region amino acid | 110 |
| | LC constant region nucleic acid | 111 |
| | LC constant region amino acid | 112 |
| PA2LR3-P3B8 (chimeric) | Heavy chain (HC) variable nucleic acid | 113 |
| | HC variable region amino acid | 114 |
| | HC constant region nucleic acid | 115 |
| | HC constant region amino acid | 116 |
| | Light chain (LC) variable region nucleic acid | 117 |
| | LC variable region amino acid | 118 |
| | LC constant region nucleic acid | 119 |
| | LC constant region amino acid | 120 |
| PA2LR3-P3F8 (chimeric) | Heavy chain (HC) variable nucleic acid | 121 |
| | HC variable region amino acid | 122 |
| | HC constant region nucleic acid | 123 |
| | HC constant region amino acid | 124 |
| | Light chain (LC) variable region nucleic acid | 125 |
| | LC variable region amino acid | 126 |
| | LC constant region nucleic acid | 127 |
| | LC constant region amino acid | 128 |
| PA2LR3-P4C6 (chimeric) | Heavy chain (HC) variable nucleic acid | 129 |
| | HC variable region amino acid | 130 |
| | HC constant region nucleic acid | 131 |
| | HC constant region amino acid | 132 |
| | Light chain (LC) variable region nucleic acid | 133 |
| | LC variable region amino acid | 134 |
| | LC constant region nucleic acid | 135 |
| | LC constant region amino acid | 136 |
| PA2LR3-P4G10 (chimeric) | Heavy chain (HC) variable nucleic acid | 137 |
| | HC variable region amino acid | 138 |
| | HC constant region nucleic acid | 139 |
| | HC constant region amino acid | 140 |
| | Light chain (LC) variable region nucleic acid | 141 |
| | LC variable region amino acid | 142 |
| | LC constant region nucleic acid | 143 |
| | LC constant region amino acid | 144 |
| PA2LR3-P5E7 (chimeric) | Heavy chain (HC) variable nucleic acid | 145 |
| | HC variable region amino acid | 146 |
| | HC constant region nucleic acid | 147 |
| | HC constant region amino acid | 148 |
| | Light chain (LC) variable region nucleic acid | 149 |
| | LC variable region amino acid | 150 |
| | LC constant region nucleic acid | 151 |
| | LC constant region amino acid | 152 |
| PA2LR3-P6D7 (chimeric) | Heavy chain (HC) variable nucleic acid | 153 |
| | HC variable region amino acid | 154 |
| | HC constant region nucleic acid | 155 |
| | HC constant region amino acid | 156 |
| | Light chain (LC) variable region nucleic acid | 157 |
| | LC variable region amino acid | 158 |
| | LC constant region nucleic acid | 159 |
| | LC constant region amino acid | 160 |
| PA2R3-P1A7 (chimeric) | Heavy chain (HC) variable nucleic acid | 161 |
| | HC variable region amino acid | 162 |
| | HC constant region nucleic acid | 163 |
| | HC constant region amino acid | 164 |
| | Light chain (LC) variable region nucleic acid | 165 |
| | LC variable region amino acid | 166 |
| | LC constant region nucleic acid | 167 |
| | LC constant region amino acid | 168 |
| PA2R3-P1F1 (chimeric) | Heavy chain (HC) variable nucleic acid | 169 |
| | HC variable region amino acid | 170 |
| | HC constant region nucleic acid | 171 |
| | HC constant region amino acid | 172 |
| | Light chain (LC) variable region nucleic acid | 173 |
| | LC variable region amino acid | 174 |
| | LC constant region nucleic acid | 175 |
| | LC constant region amino acid | 176 |
| PA13R3-P3A7 (chimeric) | Heavy chain (HC) variable nucleic acid | 177 |
| | HC variable region amino acid | 178 |
| | HC constant region nucleic acid | 179 |
| | HC constant region amino acid | 180 |
| | Light chain (LC) variable region nucleic acid | 181 |
| | LC variable region amino acid | 182 |
| | LC constant region nucleic acid | 183 |
| | LC constant region amino acid | 184 |
| PA13R3-P3C3 (chimeric) | Heavy chain (HC) variable nucleic acid | 185 |
| | HC variable region amino acid | 186 |
| | HC constant region nucleic acid | 187 |
| | HC constant region amino acid | 188 |
| | Light chain (LC) variable region nucleic acid | 189 |
| | LC variable region amino acid | 190 |
| | LC constant region nucleic acid | 191 |
| | LC constant region amino acid | 192 |
| PA13R3-P3D10 (chimeric) | Heavy chain (HC) variable nucleic acid | 193 |
| | HC variable region amino acid | 194 |
| | HC constant region nucleic acid | 195 |
| | HC constant region amino acid | 196 |
| | Light chain (LC) variable region nucleic acid | 197 |
| | LC variable region amino acid | 198 |
| | LC constant region nucleic acid | 199 |
| | LC constant region amino acid | 200 |
| PA13R3-P3D11 (chimeric) | Heavy chain (HC) variable nucleic acid | 201 |
| | HC variable region amino acid | 202 |
| | HC constant region nucleic acid | 203 |
| | HC constant region amino acid | 204 |
| | Light chain (LC) variable region nucleic acid | 205 |
| | LC variable region amino acid | 206 |
| | LC constant region nucleic acid | 207 |
| | LC constant region amino acid | 208 |
| PA13R3-P3F6 (chimeric) | Heavy chain (HC) variable nucleic acid | 209 |
| | HC variable region amino acid | 210 |
| | HC constant region nucleic acid | 211 |
| | HC constant region amino acid | 212 |
| | Light chain (LC) variable region nucleic acid | 213 |
| | LC variable region amino acid | 214 |
| | LC constant region nucleic acid | 215 |
| | LC constant region amino acid | 216 |
| PA13R3-P4C4 (chimeric) | Heavy chain (HC) variable nucleic acid | 217 |
| | HC variable region amino acid | 218 |
| | HC constant region nucleic acid | 219 |
| | HC constant region amino acid | 220 |
| | Light chain (LC) variable region nucleic acid | 221 |
| | LC variable region amino acid | 222 |
| | LC constant region nucleic acid | 223 |
| | LC constant region amino acid | 224 |
| PA13R3-P4F8 (chimeric) | Heavy chain (HC) variable nucleic acid | 225 |
| | HC variable region amino acid | 226 |
| | HC constant region nucleic acid | 227 |
| | HC constant region amino acid | 228 |
| | Light chain (LC) variable region nucleic acid | 229 |
| | LC variable region amino acid | 230 |
| | LC constant region nucleic acid | 231 |
| | LC constant region amino acid | 232 |
| PA13R3-P4G11 (chimeric) | Heavy chain (HC) variable nucleic acid | 233 |
| | HC variable region amino acid | 234 |
| | HC constant region nucleic acid | 235 |
| | HC constant region amino acid | 236 |
| | Light chain (LC) variable region nucleic acid | 237 |
| | LC variable region amino acid | 238 |
| | LC constant region nucleic acid | 239 |
| | LC constant region amino acid | 240 |
| PA13R3-P4H10 (chimeric) | Heavy chain (HC) variable nucleic acid | 241 |
| | HC variable region amino acid | 242 |
| | HC constant region nucleic acid | 243 |
| | HC constant region amino acid | 244 |
| | Light chain (LC) variable region nucleic acid | 245 |
| | LC variable region amino acid | 246 |
| | LC constant region nucleic acid | 247 |
| | LC constant region amino acid | 248 |
| PA15R3-P3A6 (chimeric) | Heavy chain (HC) variable nucleic acid | 249 |
| | HC variable region amino acid | 250 |
| | HC constant region nucleic acid | 251 |
| | HC constant region amino acid | 252 |
| | Light chain (LC) variable region nucleic acid | 253 |
| | LC variable region amino acid | 254 |
| | LC constant region nucleic acid | 255 |
| | LC constant region amino acid | 256 |
| PA15R3-P3A7(chimeric) | Heavy chain (HC) variable nucleic acid | 257 |
| | HC variable region amino acid | 258 |
| | HC constant region nucleic acid | 259 |
| | HC constant region amino acid | 260 |
| | Light chain (LC) variable region nucleic acid | 261 |
| | LC variable region amino acid | 262 |
| | LC constant region nucleic acid | 263 |
| | LC constant region amino acid | 264 |
| PA15R3-P3C9 (chimeric) | Heavy chain (HC) variable nucleic acid | 265 |
| | HC variable region amino acid | 266 |
| | HC constant region nucleic acid | 267 |
| | HC constant region amino acid | 268 |
| | Light chain (LC) variable region nucleic acid | 269 |
| | LC variable region amino acid | 270 |
| | LC constant region nucleic acid | 271 |
| | LC constant region amino acid | 272 |
| PA16R3-P2G6 (chimeric) | Heavy chain (HC) variable nucleic acid | 273 |
| | HC variable region amino acid | 274 |
| | HC constant region nucleic acid | 275 |
| | HC constant region amino acid | 276 |
| | Light chain (LC) variable region nucleic acid | 277 |
| | LC variable region amino acid | 278 |
| | LC constant region nucleic acid | 279 |
| | LC constant region amino acid | 280 |
| PA16R3-P1A6 (chimeric) | Heavy chain (HC) variable nucleic acid | 281 |
| | HC variable region amino acid | 282 |
| | HC constant region nucleic acid | 283 |
| | HC constant region amino acid | 284 |
| | Light chain (LC) variable region nucleic acid | 285 |
| | LC variable region amino acid | 286 |
| | LC constant region nucleic acid | 287 |
| | LC constant region amino acid | 288 |
| PA16R3-P1B5 (chimeric) | Heavy chain (HC) variable nucleic acid | 289 |
| | HC variable region amino acid | 290 |
| | HC constant region nucleic acid | 291 |
| | HC constant region amino acid | 292 |
| | Light chain (LC) variable region nucleic acid | 293 |
| | LC variable region amino acid | 294 |
| | LC constant region nucleic acid | 295 |
| | LC constant region amino acid | 296 |
| PA16R3-P1E5 (chimeric) | Heavy chain (HC) variable nucleic acid | 297 |
| | HC variable region amino acid | 298 |
| | HC constant region nucleic acid | 299 |
| | HC constant region amino acid | 300 |
| | Light chain (LC) variable region nucleic acid | 301 |
| | LC variable region amino acid | 302 |
| | LC constant region nucleic acid | 303 |
| | LC constant region amino acid | 304 |
| PA16R3-P1H5 (chimeric) | Heavy chain (HC) variable nucleic acid | 305 |
| | HC variable region amino acid | 306 |
| | HC constant region nucleic acid | 307 |
| | HC constant region amino acid | 308 |
| | Light chain (LC) variable region nucleic acid | 309 |
| | LC variable region amino acid | 310 |
| | LC constant region nucleic acid | 311 |
| | LC constant region amino acid | 312 |
| PA18R3-P1D8 (chimeric) | Heavy chain (HC) variable nucleic acid | 313 |
| | HC variable region amino acid | 314 |
| | HC constant region nucleic acid | 315 |
| | HC constant region amino acid | 316 |
| | Light chain (LC) variable region nucleic acid | 317 |
| | LC variable region amino acid | 318 |
| | LC constant region nucleic acid | 319 |
| | LC constant region amino acid | 320 |
| PA18R3-P1E5 (chimeric) | Heavy chain (HC) variable nucleic acid | 321 |
| | HC variable region amino acid | 322 |
| | HC constant region nucleic acid | 323 |
| | HC constant region amino acid | 324 |
| | Light chain (LC) variable region nucleic acid | 325 |
| | LC variable region amino acid | 326 |
| | LC constant region nucleic acid | 327 |
| | LC constant region amino acid | 328 |
| PA18R3-P1H5 (chimeric) | Heavy chain (HC) variable nucleic acid | 329 |
| | HC variable region amino acid | 330 |
| | HC constant region nucleic acid | 331 |
| | HC constant region amino acid | 332 |
| | Light chain (LC) variable region nucleic acid | 333 |
| | LC variable region amino acid | 334 |
| | LC constant region nucleic acid | 335 |
| | LC constant region amino acid | 336 |

TABLE 3
Sequences of exemplary humanized antibodies

| Name/ | SEQ | |
|---|---|---|
| sequence | ID | |
| description | NO | Sequence |
| Humanized | 337 | EIVLTQSPATLSLSPGERATLSCRASQSVSSSYLHWYQQKPGQAPRLLIYSTSN |
| P1C4 light | | LASGIPARFSGSGSGTDFTLTISRLEPEDFAVYYCHQYHRSPPTFGQGTKVEIK |
| chain | | |
| variable | | |
| region | | |
| Humanized | 338 | EIVLTQSPATLSLSPGERATLSCRASQSVSSSYLHWYQQKPGQAPRLLIYSTSN |
| P1C4 full | | LASGIPARFSGSGSGTDFTLTISRLEPEDFAVYYCHQYHRSPPTFGQGTKVEIK |
| light chain | | RTVAAPSVFIFPPSDEQLKSGTASVVCLLNNFYPREAKVQWKVDNALQSGNSQE |
| | | SVTEQDSKDSTYSLSSTLTLSKADYEKHKVYACEVTHQGLSSPVTKSFNRGEC |
| Humanized | 339 | QVQLVQSGAEVKKPGSSVKVSCKASGGTFSYYWMNWVRQAPGQGLEWMGQIYPG |
| P1C4-H0 | | DGETKYAQKFQGRVTITADKSTSTAYMELSSLRSEDTAVYYCARSHGNYLPYWG |
| heavy chain | | QGTLVTVSS |
| variable | | |
| region | | |
| Humanized | 340 | QVQLVQSGAEVKKPGSSVKVSCKASGYEFSYYWMNWVRQAPGQGLEWMGQIYPG |
| P1C4-H2 | | DGETKYAQKFQGRVTITADKSTSTAYMELSSLRSEDTAVYYCARSHGNYLPYWG |
| heavy chain | | QGTLVTVSS |
| variable | | |
| region | | |
| Humanized | 341 | QVQLVQSGAEVKKPGSSVKVSCKASGYEFSYYWMNWVRQAPGQGLEWMGQIYPG |
| P1C4-H4 | | DGETKYNGKFQGRVTITADKSTSTAYMELSSLRSEDTAVYYCARSHGNYLPYWG |
| heavy chain | | QGTLVTVSS |
| variable | | |
| region | | |
| Humanized | 342 | ASTKGPSVFPLAPSSKSTSGGTAALGCLVKDYFPEPVTVSWNSGALTSGVHTFP |
| P1C4 heavy | | AVLQSSGLYSLSSVVTVPSSSLGTQTYICNVNHKPSNTKVDKKVEPKSCDKTHT |
| chain | | CPPCPAPELLGGPSVFLFPPKPKDTLMISRTPEVTCVVVDVSHEDPEVKFNWYV |
| constant | | DGVEVHNAKTKPREEQYNSTYRVVSVLTVLHQDWLNGKEYKCKVSNKALPAPIE |
| region | | KTISKAKGQPREPQVYTLPPSRDELTKNQVSLTCLVKGFYPSDIAVEWESNGQP |
| | | ENNYKTTPPVLDSDGSFFLYSKLTVDKSRWQQGNVFSCSVMHEALHNHYTQKSL |
| | | SLSPGK |
| Humanized | 343 | QVQLVQSGAEVKKPGSSVKVSCKASGGTFSYYWMNWVRQAPGQGLEWMGQIYPG |
| P1C4 H0 | | DGETKYAQKFQGRVTITADKSTSTAYMELSSLRSEDTAVYYCARSHGNYLPYWG |
| IgG2-4 | | QGTLVTVSSASTKGPSVFPLAPCSRSTSESTAALGCLVKDYFPEPVTVSWNSGA |
| Hybrid | | LTSGVHTFPAVLQSSGLYSLSSVVTVPSSNFGTQTYTCNVDHKPSNTKVDKTVE |
| | | RKCCVECPPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEALHNH |
| | | YTQKSLSLSLGK |
| Humanized | 344 | QVQLVQSGAEVKKPGSSVKVSCKASGGTFSYYWMNWVRQAPGQGLEWMGQIYPG |
| P1C4 H0 | | DGETKYAQKFQGRVTITADKSTSTAYMELSSLRSEDTAVYYCARSHGNYLPYWG |
| IgG2A330S/ | | QGTLVTVSSASTKGPSVFPLAPCSRSTSESTAALGCLVKDYFPEPVTVSWNSGA |
| P331S | | LTSGVHTFPAVLQSSGLYSLSSVVTVPSSNFGTQTYTCNVDHKPSNTKVDKTVE |
| | | RKCCVECPPCPAPPVAGPSVFLFPPKPKDTLMISRTPEVTCVVVDVSHEDPEVQ |
| | | FNWYVDGVEVHNAKTKPREEQFNSTFRVVSVLTVVHQDWLNGKEYKCKVSNKGL |
| | | PSSIEKTISKTKGQPREPQVYTLPPSREEMTKNQVSLTCLVKGFYPSDIAVEWE |
| | | SNGQPENNYKTTPPMLDSDGSFFLYSKLTVDKSRWQQGNVFSCSVMHEALHNHY |
| | | TQKSLSLSPGK |
| Humanized | 345 | QVQLVQSGAEVKKPGSSVKVSCKASGGTFSYYWMNWVRQAPGQGLEWMGQIYPG |
| P1C4 H0 | | DGETKYAQKFQGRVTITADKSTSTAYMELSSLRSEDTAVYYCARSHGNYLPYWG |
| IgG4S228P | | QGTLVTVSSASTKGPSVFPLAPCSRSTSESTAALGCLVKDYFPEPVTVSWNSGA |
| | | LTSGVHTFPAVLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEALHNH |
| | | YTQKSLSLSLGK |
| Humanized | 346 | QVQLVQSGAEVKKPGSSVKVSCKASGYEFSYYWMNWVRQAPGQGLEWMGQIYPG |
| P1C4 H2 | | DGETKYAQKFQGRVTITADKSTSTAYMELSSLRSEDTAVYYCARSHGNYLPYWG |
| IgG2-4 | | QGTLVTVSSASTKGPSVFPLAPCSRSTSESTAALGCLVKDYFPEPVTVSWNSGA |
| Hybrid | | LTSGVHTFPAVLQSSGLYSLSSVVTVPSSNFGTQTYTCNVDHKPSNTKVDKTVE |
| | | RKCCVECPPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEALHNH |
| | | YTQKSLSLSLGK |
| Humanized | 347 | QVQLVQSGAEVKKPGSSVKVSCKASGYEFSYYWMNWVRQAPGQGLEWMGQIYPG |
| P1C4 H2 | | DGETKYAQKFQGRVTITADKSTSTAYMELSSLRSEDTAVYYCARSHGNYLPYWG |
| IgG2A330S/ | | QGTLVTVSSASTKGPSVFPLAPCSRSTSESTAALGCLVKDYFPEPVTVSWNSGA |
| P331S | | LTSGVHTFPAVLQSSGLYSLSSVVTVPSSNFGTQTYTCNVDHKPSNTKVDKTVE |
| | | RKCCVECPPCPAPPVAGPSVFLFPPKPKDTLMISRTPEVTCVVVDVSHEDPEVQ |
| | | FNWYVDGVEVHNAKTKPREEQFNSTFRVVSVLTVVHQDWLNGKEYKCKVSNKGL |
| | | PSSIEKTISKTKGQPREPQVYTLPPSREEMTKNQVSLTCLVKGFYPSDIAVEWE |
| | | SNGQPENNYKTTPPMLDSDGSFFLYSKLTVDKSRWQQGNVFSCSVMHEALHNHY |
| | | TQKSLSLSPGK |
| Humanized | 348 | QVQLVQSGAEVKKPGSSVKVSCKASGYEFSYYWMNWVRQAPGQGLEWMGQIYPG |
| P1C4 H2 | | DGETKYAQKFQGRVTITADKSTSTAYMELSSLRSEDTAVYYCARSHGNYLPYWG |
| IgG4S228P | | QGTLVTVSSASTKGPSVFPLAPCSRSTSESTAALGCLVKDYFPEPVTVSWNSGA |
| | | LTSGVHTFPAVLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEALHNH |
| | | YTQKSLSLSLGK |
| Humanized | 349 | QVQLVQSGAEVKKPGSSVKVSCKASGYEFSYYWMNWVRQAPGQGLEWMGQIYPG |
| P1C4 H4 | | DGETKYNGKFQGRVTITADKSTSTAYMELSSLRSEDTAVYYCARSHGNYLPYWG |
| IgG2-4 | | QGTLVTVSSASTKGPSVFPLAPCSRSTSESTAALGCLVKDYFPEPVTVSWNSGA |
| Hybrid | | LTSGVHTFPAVLQSSGLYSLSSVVTVPSSNFGTQTYTCNVDHKPSNTKVDKTVE |
| | | RKCCVECPPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEALHNH |
| | | YTQKSLSLSLGK |
| Humanized | 350 | QVQLVQSGAEVKKPGSSVKVSCKASGYEFSYYWMNWVRQAPGQGLEWMGQIYPG |
| P1C4 H4 | | DGETKYNGKFQGRVTITADKSTSTAYMELSSLRSEDTAVYYCARSHGNYLPYWG |
| IgG2A330S/ | | QGTLVTVSSASTKGPSVFPLAPCSRSTSESTAALGCLVKDYFPEPVTVSWNSGA |
| P331S | | LTSGVHTFPAVLQSSGLYSLSSVVTVPSSNFGTQTYTCNVDHKPSNTKVDKTVE |
| | | RKCCVECPPCPAPPVAGPSVFLFPPKPKDTLMISRTPEVTCVVVDVSHEDPEVQ |
| | | FNWYVDGVEVHNAKTKPREEQFNSTFRVVSVLTVVHQDWLNGKEYKCKVSNKGL |
| | | PSSIEKTISKTKGQPREPQVYTLPPSREEMTKNQVSLTCLVKGFYPSDIAVEWE |
| | | SNGQPENNYKTTPPMLDSDGSFFLYSKLTVDKSRWQQGNVFSCSVMHEALHNHY |
| | | TQKSLSLSPGK |
| Humanized | 351 | QVQLVQSGAEVKKPGSSVKVSCKASGYEFSYYWMNWVRQAPGQGLEWMGQIYPG |
| P1C4 H4 | | DGETKYNGKFQGRVTITADKSTSTAYMELSSLRSEDTAVYYCARSHGNYLPYWG |
| IgG4S228P | | QGTLVTVSSASTKGPSVFPLAPCSRSTSESTAALGCLVKDYFPEPVTVSWNSGA |
| | | LTSGVHTFPAVLQSSGLYSLSSVVTVPSSSLGTKTYTCNVDHKPSNTKVDKRVE |
| | | SKYGPPCPPCPAPEFLGGPSVFLFPPKPKDTLMISRTPEVTCVVVDVSQEDPEV |
| | | QFNWYVDGVEVHNAKTKPREEQFNSTYRVVSVLTVLHQDWLNGKEYKCKVSNKG |
| | | LPSSIEKTISKAKGQPREPQVYTLPPSQEEMTKNQVSLTCLVKGFYPSDIAVEW |
| | | ESNGQPENNYKTTPPVLDSDGSFFLYSRLTVDKSRWQEGNVFSCSVMHEALHNH |
| | | YTQKSLSLSLGK |

TABLE 4
Design of Fc variants

| Antibody/ | | |
|---|---|---|
| Fragment | | |
| Name or | | SEQ |
| Sequence | | ID |
| Description | Sequence | NO: |
| Humanized | QVQLVQSGAEVKKPGSSVKVSCKASGYEFSYYWMNWVRQAPGQGLEWMGQI | 340 |
| P1C4-H2 | YPGDGETKYAQKFQGRVTITADKSTSTAYMELSSLRSEDTAVYYCARSHGN | |
| heavy chain | YLPYWGQGTLVTVSS | |
| variable | | |
| region | | |
| Humanized | QVQLVQSGAEVKKPGSSVKVSCKASGYEFSYYWMNWVRQAPGQGLEWMGQI | 341 |
| P1C4-H4 | YPGDGETKYNGKFQGRVTITADKSTSTAYMELSSLRSEDTAVYYCARSHGN | |
| heavy chain | YLPYWGQGTLVTVSS | |
| variable | | |
| region | | |
| Humanized | EIVLTQSPATLSLSPGERATLSCRASQSVSSSYLHWYQQKPGQAPRLLIYS | 338 |
| P1C4 full | TSNLASGIPARFSGSGSGTDFTLTISRLEPEDFAVYYCHQYHRSPPTFGQG | |
| light chain | TKVEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSS | |
| | PVTKSFNRGEC | |
| IgG2 heavy | ASTKGPSVFPLAPCSRSTSESTAALGCLVKDYFPEPVTVSWNSGALTSGVH | 433 |
| chain | TFPAVLQSSGLYSLSSVVTVPSSNFGTQTYTCNVDHKPSNTKVDKTVERKC | |
| constant | CVECPPCPAPPVAGPSVFLFPPKPKDTLMISRTPEVTCVVVDVSHEDPEVQ | |
| region | FNWYVDGVEVHNAKTKPREEQFNSTFRVVSVLTVVHQDWLNGKEYKCKVSN | |
| | KGLPSSIEKTISKTKGQPREPQVYTLPPSREEMTKNQVSLTCLVKGFYPSD | |
| | IAVEWESNGQPENNYKTTPPMLDSDGSFFLYSKLTVDKSRWQQGNVFSCSV | |
| | MHEALHNHYTQKSLSLSPGK | |
| IgG4 heavy | ASTKGPSVFPLAPCSRSTSESTAALGCLVKDYFPEPVTVSWNSGALTSGVH | 434 |
| chain | TFPAVLQSSGLYSLSSVVTVPSSSLGTKTYTCNVDHKPSNTKVDKRVESKY | |
| constant | GPPCPPCPAPEFLGGPSVFLFPPKPKDTLMISRTPEVTCVVVDVSQEDPEV | |
| region | QFNWYVDGVEVHNAKTKPREEQFNSTYRVVSVLTVLHQDWLNGKEYKCKVS | |
| | NKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCS | |
| | VMHEALHNHYTQKSLSLSLGK | |
| Hybrid | ASTKGPSVFPLAPCSRSTSESTAALGCLVKDYFPEPVTVSWNSGALTSGVH | 435 |
| IgG2/4 heavy | TFPAVLQSSGLYSLSSVVTVPSSNFGTQTYTCNVDHKPSNTKVDKTVERKC | |
| chain | CVECPPCPAPEFLGGPSVFLFPPKPKDTLMISRTPEVTCVVVDVSQEDPEV | |
| constant | QFNWYVDGVEVHNAKTKPREEQFNSTYRVVSVLTVLHQDWLNGKEYKCKVS | |
| region | NKGLPSSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGNVFSCS | |
| | VMHEALHNHYTQKSLSLSLGK | |
| P1C4-H2- | QVQLVQSGAEVKKPGSSVKVSCKASGYEFSYYWMNWVRQAPGQGLEWMGQI | 436 |
| IgG2 | YPGDGETKYAQKFQGRVTITADKSTSTAYMELSSLRSEDTAVYYCARSHGN | |
| | YLPYWGQGTLVTVSSASTKGPSVFPLAPCSRSTSESTAALGCLVKDYFPEP | |
| | VTVSWNSGALTSGVHTFPAVLQSSGLYSLSSVVTVPSSNFGTQTYTCNVDH | |
| | KPSNTKVDKTVERKCCVECPPCPAPPVAGPSVFLFPPKPKDTLMISRTPEV | |
| | TCVVVDVSHEDPEVQFNWYVDGVEVHNAKTKPREEQFNSTFRVVSVLTVVH | |
| | QDWLNGKEYKCKVSNKGLPSSIEKTISKTKGQPREPQVYTLPPSREEMTKN | |
| | QVSLTCLVKGFYPSDIAVEWESNGQPENNYKTTPPMLDSDGSFFLYSKLTV | |
| | DKSRWQQGNVFSCSVMHEALHNHYTQKSLSLSPGK | |
| P1C4-H2- | QVQLVQSGAEVKKPGSSVKVSCKASGYEFSYYWMNWVRQAPGQGLEWMGQI | 437 |
| IgG4 | YPGDGETKYAQKFQGRVTITADKSTSTAYMELSSLRSEDTAVYYCARSHGN | |
| | YLPYWGQGTLVTVSSASTKGPSVFPLAPCSRSTSESTAALGCLVKDYFPEP | |
| | VTVSWNSGALTSGVHTFPAVLQSSGLYSLSSVVTVPSSSLGTKTYTCNVDH | |
| | KPSNTKVDKRVESKYGPPCPPCPAPEFLGGPSVFLFPPKPKDTLMISRTPE | |
| | VTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGK | |
| P1C4-H2- | QVQLVQSGAEVKKPGSSVKVSCKASGYEFSYYWMNWVRQAPGQGLEWMGQI | 438 |
| IgG2/4 | YPGDGETKYAQKFQGRVTITADKSTSTAYMELSSLRSEDTAVYYCARSHGN | |
| | YLPYWGQGTLVTVSSASTKGPSVFPLAPCSRSTSESTAALGCLVKDYFPEP | |
| | VTVSWNSGALTSGVHTFPAVLQSSGLYSLSSVVTVPSSNFGTQTYTCNVDH | |
| | KPSNTKVDKTVERKCCVECPPCPAPEFLGGPSVFLFPPKPKDTLMISRTPE | |
| | VTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGK | |
| P1C4-H4- | QVQLVQSGAEVKKPGSSVKVSCKASGYEFSYYWMNWVRQAPGQGLEWMGQI | 439 |
| IgG2 | YPGDGETKYNGKFQGRVTITADKSTSTAYMELSSLRSEDTAVYYCARSHGN | |
| | YLPYWGQGTLVTVSSASTKGPSVFPLAPCSRSTSESTAALGCLVKDYFPEP | |
| | VTVSWNSGALTSGVHTFPAVLQSSGLYSLSSVVTVPSSNFGTQTYTCNVDH | |
| | KPSNTKVDKTVERKCCVECPPCPAPPVAGPSVFLFPPKPKDTLMISRTPEV | |
| | TCVVVDVSHEDPEVQFNWYVDGVEVHNAKTKPREEQFNSTFRVVSVLTVVH | |
| | QDWLNGKEYKCKVSNKGLPSSIEKTISKTKGQPREPQVYTLPPSREEMTKN | |
| | QVSLTCLVKGFYPSDIAVEWESNGQPENNYKTTPPMLDSDGSFFLYSKLTV | |
| | DKSRWQQGNVFSCSVMHEALHNHYTQKSLSLSPGK | |
| PIC4-H4- | QVQLVQSGAEVKKPGSSVKVSCKASGYEFSYYWMNWVRQAPGQGLEWMGQI | 440 |
| IgG4 | YPGDGETKYNGKFQGRVTITADKSTSTAYMELSSLRSEDTAVYYCARSHGN | |
| | YLPYWGQGTLVTVSSASTKGPSVFPLAPCSRSTSESTAALGCLVKDYFPEP | |
| | VTVSWNSGALTSGVHTFPAVLQSSGLYSLSSVVTVPSSSLGTKTYTCNVDH | |
| | KPSNTKVDKRVESKYGPPCPPCPAPEFLGGPSVFLFPPKPKDTLMISRTPE | |
| | VTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | 441 |
| | VDKSRWQEGNVFSCSVMHEALHNHYTQKSLSLSLGK | |
| P1C4-H4- | QVQLVQSGAEVKKPGSSVKVSCKASGYEFSYYWMNWVRQAPGQGLEWMGQI | |
| IgG2/4 | YPGDGETKYNGKFQGRVTITADKSTSTAYMELSSLRSEDTAVYYCARSHGN | |
| | YLPYWGQGTLVTVSSASTKGPSVFPLAPCSRSTSESTAALGCLVKDYFPEP | |
| | VTVSWNSGALTSGVHTFPAVLQSSGLYSLSSVVTVPSSNFGTQTYTCNVDH | |
| | KPSNTKVDKTVERKCCVECPPCPAPEFLGGPSVFLFPPKPKDTLMISRTPE | |
| | VTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGK | |
| P1C4-Lc | EIVLTQSPATLSLSPGERATLSCRASQSVSSSYLHWYQQKPGQAPRLLIYS | 442 |
| humanized | TSNLASGIPARFSGSGSGTDFTLTISRLEPEDFAVYYCHQYHRSPPTFGQG | |
| | TKVEIKRTVAAPSVFIFPPSDEQLKSGTASVVCLLNNFYPREAKVQWKVDN | |
| | ALQSGNSQESVTEQDSKDSTYSLSSTLTLSKADYEKHKVYACEVTHQGLSS | |
| | PVTKSFNRGEC | |

TABLE 5
Allowable mutations PA13R3-P1C4 Heavy Chain CDRs

| CDR | Position | Allowable change (s) |
|---|---|---|
| HCDR1 | Y1 | H, W |
| | Y2 | F, I, K, N |
| | W3 | A, F |
| | M4 | A, E, F, L, N, Q, T, V |
| | N5 | I, K, L, S, W |
| HCDR2 | Q1 | D, E |
| | I2 | A, D, E, F, G, H, K, L, N, Q, R, S, T, W, Y |
| | Y3 | F |
| | P4 | A, F, H, K |
| | G5 | A, C, D, E, F, H, I, K, L, M, Q, R, S, T, V, W, Y |
| | D6 | I, L, M, N, P, Q, V, W, Y |
| | G7 | A, D, E, F, H, I, K, L, M, N, P, Q, R, S, T, V, Y |
| | E8 | A, D, M, Q, V, Y |
| | T9 | A, D, E, F, G, H, I, K, Q, R, S, T, W, Y |
| | K10 | D, E, H, I, L, M, N, P, Q, R, S, T, V, W, Y |
| | Y11 | C, D, E, F, G, H, I, L, N, P, Q, R, T, W |
| HCDR3 | S1 | N, T, Y |
| | Y5 | A, C, F, H, N, S |
| | L6 | D, E, F, G, H, I, K, M, N, Q, S, W, Y |
| | P7 | A, E, F, G, H, KL, Q, R, S, V, W, Y |
| | Y8 | A, D, E, F, G, H, I, K, L, M, R, S, V |

TABLE 6
Allowable mutations PA13R3-P1C4 Light Chain CDRs

| CDR | Position | Allowable change (s) |
|---|---|---|
| LCDR1 | S1 | A, D, F, L, M, R, T, V, W, Y |
| | S2 | A, D, E, F, G, H, K, M, N, P, Q, V, W |
| | Y3 | F |
| | L4 | F, H, I, K, N, P, Q, R, S, T, W, Y |
| LCDR2 | S1 | A, H, N, R |
| | T2 | A, D, F, G, H, L, M, S, V, W, Y |
| | S3 | D, F, H, I, K, LN, Q, Y |
| | N4 | D, E, F, G, H, I, K, Q, R, S, V |
| | L5 | D, E, F, G, H, I, K, M, N, P, Q, V, W, Y |
| | A6 | D, F, G, I, K, Q, R, S, V, W |
| | S7 | A, D, F, G, H, K, L, R, T, V, W |
| | G8 | A, F, I, N, P, R, S, T, V, Y |
| LCDR3 | H1 | A, C, D, E, F, I, K, L, N, R, S, T, V, W, Y |
| | Q2 | A, C, E, G, N, S, T, V |
| | Y3 | A, F, G, H, Q, W |
| | H4 | A, E, G, K, L, N, Q, S, T, V, W |
| | R5 | A, C, D, E, F, I, L, M, N, Q, V, W, Y |
| | S6 | A, C, E, F, G, I, M, P, R, T, V, W, Y |
| | P7 | K, W, Y |
| | P8 | D, H |
| | T9 | D, F, G, I, L, M, N, Q, R, S, V, Y |

TABLE 7
Sequences of allowable mutations within PA13R3-P1C4 CDRs

| Source Sequence | | | |
|---|---|---|---|
| Description | Sequence | Mutation | SEQ ID NO |
| Heavy chain | YYWMN | | 352 |
| CDR1 sequence | HYWMN | Y1→H | 443 |
| | IYWMN | Y1→I | 444 |
| | WYWMN | Y1→W | 445 |
| | YFWMN | Y2→F | 446 |
| | YKWMN | Y2→K | 447 |
| | YNWMN | Y2→N | 448 |
| | YYAMN | W3→A | 449 |
| | YYFMN | W3→F | 450 |
| | YYWAN | M4→A | 451 |
| | YYWEN | M4→E | 452 |
| | YYWFN | M4→F | 453 |
| | YYWLN | M4→L | 454 |
| | YYWNN | M4→N | 455 |
| | YYWQN | M4→Q | 456 |
| | YYWTN | M4→T | 457 |
| | YYWVN | M4→V | 458 |
| | YYWMI | N5→I | 459 |
| | YYWMK | N5→K | 460 |
| | YYWML | N5→L | 461 |
| | YYWMS | N5→S | 462 |
| | YYWMW | N5→W | 463 |
| Heavy chain | QIYPGDGETKY | | 353 |
| CDR2 sequence | DIYPGDGETKY | Q1→D | 464 |
| | EIYPGDGETKY | Q1→E | 465 |
| | QAYPGDGETKY | I2→A | 466 |
| | QDYPGDGETKY | I2→D | 467 |
| | QEYPGDGETKY | I2→E | 468 |
| | QFYPGDGETKY | I2→F | 469 |
| | QGYPGDGETKY | I2→G | 470 |
| | QHYPGDGETKY | I2→H | 471 |
| | QKYPGDGETKY | I2→K | 472 |
| | QLYPGDGETKY | I2→L | 473 |
| | QNYPGDGETKY | I2→N | 474 |
| | QQYPGDGETKY | I2→Q | 475 |
| | QRYPGDGETKY | I2→R | 476 |
| | QSYPGDGETKY | I2→S | 477 |
| | QTYPGDGETKY | I2→T | 478 |
| | QWYPGDGETKY | I2→W | 479 |
| | QYYPGDGETKY | I2→Y | 480 |
| | QIFPGDGETKY | Y3→F | 481 |
| | QIYAGDGETKY | P4→A | 482 |
| | QIYFGDGETKY | P4→F | 483 |
| | QIYHGDGETKY | P4→H | 484 |
| | QIYKGDGETKY | P4→K | 485 |
| | QIYPADGETKY | G5→A | 486 |
| | QIYPCDGETKY | G5→C | 487 |
| | QIYPDDGETKY | G5→D | 488 |
| | QIYPEDGETKY | G5→E | 489 |
| | QIYPFDGETKY | G5→F | 490 |
| | QIYPHDGETKY | G5→H | 49 |
| | QIYPIDGETKY | G5→I | 492 |
| | QIYPKDGETKY | G5→K | 493 |
| | QIYPLDGETKY | G5→L | 494 |
| | QIYPMDGETKY | G5→M | 495 |
| | QIYPQDGETKY | G5→Q | 496 |
| | QIYPRDGETKY | G5→R | 497 |
| | QIYPSDGETKY | G5→S | 498 |
| | QIYPTDGETKY | G5→T | 499 |
| | QIYPVDGETKY | G5→V | 500 |
| | QIYPWDGETKY | G5→W | 501 |
| | QIYPYDGETKY | G5→Y | 502 |
| | QIYPGIGETKY | D6→I | 503 |
| | QIYPGLGETKY | D6→L | 504 |
| | QIYPGMGETKY | D6→M | 505 |
| | QIYPGNGETKY | D6→N | 506 |
| | QIYPGPGETKY | D6→P | 507 |
| | QIYPGQGETKY | D6→Q | 508 |
| | QIYPGVGETKY | D6→V | 509 |
| | QIYPGWGETKY | D6→W | 510 |
| | QIYPGYGETKY | D6→Y | 511 |
| | QIYPGDAETKY | G7→A | 512 |
| | QIYPGDDETKY | G7→D | 513 |
| | QIYPGDEETKY | G7→E | 514 |
| | QIYPGDFETKY | G7→F | 515 |
| | QIYPGDHETKY | G7→H | 516 |
| | QIYPGDIETKY | G7→I | 517 |
| | QIYPGDKETKY | G7→K | 518 |
| | QIYPGDLETKY | G7→L | 519 |
| | QIYPGDMETKY | G7→M | 520 |
| | QIYPGDNETKY | G7→N | 521 |
| | QIYPGDPETKY | G7→P | 522 |
| | QIYPGDQETKY | G7→Q | 523 |
| | QIYPGDRETKY | G7→R | 524 |
| | QIYPGDSETKY | G7→S | 525 |
| | QIYPGDTETKY | G7→T | 526 |
| | QIYPGDVETKY | G7→V | 527 |
| | QIYPGDYETKY | G7→Y | 528 |
| | QIYPGDGATKY | E8→A | 529 |
| | QIYPGDGDTKY | E8→D | 530 |
| | QIYPGDGMTKY | E8→M | 531 |
| | QIYPGDGQTKY | E8→Q | 532 |
| | QIYPGDGVTKY | E8→V | 533 |
| | QIYPGDGYTKY | E8→Y | 534 |
| | QIYPGDGEAKY | T9→A | 535 |
| | QIYPGDGEDKY | T9→D | 536 |
| | QIYPGDGEEKY | T9→E | 537 |
| | QIYPGDGEFKY | T9→F | 538 |
| | QIYPGDGEGKY | T9→G | 539 |
| | QIYPGDGEHKY | T9→H | 540 |
| | QIYPGDGEIKY | T9→I | 541 |
| | QIYPGDGEKKY | T9→K | 542 |
| | QIYPGDGEQKY | T9→Q | 543 |
| | QIYPGDGERKY | T9→R | 544 |
| | QIYPGDGESKY | T9→S | 545 |
| | QIYPGDGETKY | T9→T | 546 |
| | QIYPGDGEVKY | T9→W | 547 |
| | QIYPGDGEYKY | T9→Y | 548 |
| | QIYPGDGETDY | K10→D | 549 |
| | QIYPGDGETEY | K10→E | 550 |
| | QIYPGDGETHY | K10→H | 551 |
| | QIYPGDGETIY | K10→I | 552 |
| | QIYPGDGETLY | K10→L | 553 |
| | QIYPGDGETMY | K10→M | 554 |
| | QIYPGDGETNY | K10→N | 555 |
| | QIYPGDGETPY | K10→P | 556 |
| | QIYPGDGETQY | K10→Q | 557 |
| | QIYPGDGETRY | K10→R | 558 |
| | QIYPGDGETSY | K10→S | 559 |
| | QIYPGDGETTY | K10→T | 560 |
| | QIYPGDGETVY | K10→V | 561 |
| | QIYPGDGETWY | K10→W | 562 |
| | QIYPGDGETYY | K10→Y | 563 |
| | QIYPGDGETKC | Y11→C | 564 |
| | QIYPGDGETKD | Y11→D | 565 |
| | QIYPGDGETKE | Y11→E | 566 |
| | QIYPGDGETKF | Y11→F | 567 |
| | QIYPGDGETKG | Y11→G | 568 |
| | QIYPGDGETKH | Y11→H | 569 |
| | QIYPGDGETKI | Y11→I | 570 |
| | QIYPGDGETKL | Y11→L | 571 |
| | QIYPGDGETKN | Y11→N | 572 |
| | QIYPGDGETKP | Y11→P | 573 |
| | QIYPGDGETKQ | Y11→Q | 574 |
| | QIYPGDGETKR | Y11→R | 575 |
| | QIYPGDGETKT | Y11→T | 576 |
| | QIYPGDGETKW | Y11→W | 577 |
| Heavy chain | SHGNYLPY | | 354 |
| CDR3 sequence | NHGNYLPY | S1→N | 578 |
| | THGNYLPY | S1→T | 579 |
| | YHGNYLPY | S1→Y | 580 |
| | SHGNALPY | Y5→A | 58 |
| | SHGNCLPY | Y5→C | 582 |
| | SHGNFLPY | Y5→F | 583 |
| | SHGNHLPY | Y5→H | 584 |
| | SHGNNLPY | Y5→N | 585 |
| | SHGNSLPY | Y5→S | 586 |
| | SHGNYDPY | L6→D | 587 |
| | SHGNYEPY | L6→E | 588 |
| | SHGNYFPY | L6→F | 589 |
| | SHGNYGPY | L6→G | 590 |
| | SHGNYHPY | L6→H | 591 |
| | SHGNYIPY | L6→I | 592 |
| | SHGNYKPY | L6→K | 593 |
| | SHGNYMPY | L6→M | 594 |
| | SHGNYNPY | L6→N | 595 |
| | SHGNYQPY | L6→Q | 596 |
| | SHGNYSPY | L6→S | 597 |
| | SHGNYWPY | L6→W | 598 |
| | SHGNYYPY | L6→Y | 599 |
| | SHGNYLAY | P7→A | 600 |
| | SHGNYLEY | P7→E | 601 |
| | SHGNYLFY | P7→F | 602 |
| | SHGNYLGY | P7→G | 603 |
| | SHGNYLHY | P7→H | 604 |
| | SHGNYLKY | P7→K | 605 |
| | SHGNYLLY | P7→L | 606 |
| | SHGNYLQY | P7→Q | 607 |
| | SHGNYLRY | P7→R | 608 |
| | SHGNYLSY | P7→S | 609 |
| | SHGNYLVY | P7→V | 610 |
| | SHGNYLWY | P7→W | 611 |
| | SHGNYLYY | P7→Y | 612 |
| | SHGNYLPA | Y8→A | 613 |
| | SHGNYLPD | Y8→D | 614 |
| | SHGNYLPE | Y8→E | 615 |
| | SHGNYLPF | Y8→F | 616 |
| | SHGNYLPG | Y8→G | 617 |
| | SHGNYLPH | Y8→H | 618 |
| | SHGNYLPI | Y8→I | 619 |
| | SHGNYLPK | Y8→K | 620 |
| | SHGNYLPL | Y8→L | 621 |
| | SHGNYLPM | Y8→M | 622 |
| | SHGNYLPR | Y8→R | 623 |
| | SHGNYLPS | Y8→S | 624 |
| | SHGNYLPV | Y8→V | 625 |
| Light chain | SSYLH | | 355 |
| CDR1 sequence | ASYLH | S1→A | 626 |
| | DSYLH | S1→D | 627 |
| | FSYLH | S1→F | 628 |
| | LSYLH | S1→L | 629 |
| | MSYLH | S1→M | 630 |
| | RSYLH | S1→R | 631 |
| | TSYLH | S1→T | 632 |
| | VSYLH | S1→V | 633 |
| | WSYLH | S1→W | 634 |
| | YSYLH | S1→Y | 635 |
| | SAYLH | S2→A | 636 |
| | SDYLH | S2→D | 637 |
| | SEYLH | S2→E | 638 |
| | SFYLH | S2→F | 639 |
| | SGYLH | S2→G | 640 |
| | SHYLH | S2→H | 641 |
| | SKYLH | S2→K | 642 |
| | SMYLH | S2→M | 643 |
| | SNYLH | S2→N | 644 |
| | SPYLH | S2→P | 645 |
| | SQYLH | S2→Q | 646 |
| | SRYLH | S2→R | 833 |
| | SVYLH | S2→V | 647 |
| | SWYLH | S2→W | 648 |
| | SSFLH | Y3→F | 649 |
| | SSYFH | L4→F | 650 |
| | SSYHH | L4→H | 651 |
| | SSYIH | L4→I | 652 |
| | SSYKH | L4→K | 653 |
| | SSYNH | L4→N | 654 |
| | SSYPH | L4→P | 655 |
| | SSYQH | L4→Q | 656 |
| | SSYRH | L4→R | 657 |
| | SSYSH | L4→S | 658 |
| | SSYTH | L4→T | 659 |
| | SSYWH | L4→W | 660 |
| | SSYYH | L4→Y | 661 |
| Light chain | STSNLAS | | 356 |
| CDR2 sequence | ATSNLAS | S1→A | 662 |
| | GTSNLAS | S1→G | 834 |
| | HTSNLAS | S1→H | 663 |
| | NTSNLAS | S1→N | 664 |
| | RTSNLAS | S1→R | 665 |
| | SASNLAS | T2→A | 666 |
| | SDSNLAS | T2→D | 667 |
| | SFSNLAS | T2→F | 668 |
| | SGSNLAS | T2→G | 669 |
| | SHSNLAS | T2→H | 670 |
| | SLSNLAS | T2→L | 671 |
| | SMSNLAS | T2→M | 672 |
| | SSSNLAS | T2→S | 673 |
| | SVSNLAS | T2→V | 674 |
| | SWSNLAS | T2→W | 675 |
| | SYSNLAS | T2→Y | 676 |
| | STDNLAS | S3→D | 677 |
| | STENLAS | S3→F | 678 |
| | STHNLAS | S3→H | 679 |
| | STINLAS | S3→I | 680 |
| | STKNLAS | S3→K | 681 |
| | STLNLAS | S3→L | 682 |
| | STNNLAS | S3→N | 683 |
| | STQNLAS | S3→Q | 684 |
| | STYNLAS | S3→Y | 685 |
| | STSDLAS | N4→D | 686 |
| | STSELAS | N4→E | 687 |
| | STSFLAS | N4→F | 688 |
| | STSGLAS | N4→G | 689 |
| | STSHLAS | N4→H | 690 |
| | STSILAS | N4→I | 691 |
| | STSKLAS | N4→K | 692 |
| | STSQLAS | N4→Q | 693 |
| | STSRLAS | N4→R | 694 |
| | STSSLAS | N4→S | 695 |
| | STSVLAS | N4→V | 696 |
| | STSNDAS | L5→D | 697 |
| | STSNEAS | L5→E | 698 |
| | STSNFAS | L5→F | 699 |
| | STSNGAS | L5→G | 700 |
| | STSNHAS | L5→H | 701 |
| | STSNIAS | L5→I | 702 |
| | STSNKAS | L5→K | 703 |
| | STSNMAS | L5→M | 704 |
| | STSNNAS | L5→N | 705 |
| | STSNPAS | L5→P | 706 |
| | STSNQAS | L5→Q | 707 |
| | STSNRAS | L5→R | 835 |
| | STSNVAS | L5→V | 708 |
| | STSNWAS | L5→W | 709 |
| | STSNYAS | L5→Y | 710 |
| | STSNLDS | A6→D | 711 |
| | STSNLFS | A6→F | 712 |
| | STSNLGS | A6→G | 713 |
| | STSNLIS | A6→I | 714 |
| | STSNLKS | A6→K | 715 |
| | STSNLQS | A6→Q | 716 |
| | STSNLRS | A6→R | 717 |
| | STSNLSS | A6→S | 718 |
| | STSNLVS | A6→V | 719 |
| | STSNLWS | A6→W | 720 |
| | STSNLAA | S7→A | 721 |
| | STSNLAD | S7→D | 722 |
| | STSNLAF | S7→F | 723 |
| | STSNLAG | S7→G | 724 |
| | STSNLAH | S7→H | 725 |
| | STSNLAK | S7→K | 726 |
| | STSNLAL | S7→L | 727 |
| | STSNLAR | S7→R | 728 |
| | STSNLAT | S7→T | 729 |
| | STSNLAV | S7→V | 730 |
| | STSNLAW | S7→W | 731 |
| | STSNLASG | | 732 |
| | STSNLASA | G8→A | 733 |
| | STSNLASF | G8→F | 734 |
| | STSNLASI | G8→I | 735 |
| | STSNLASN | G8→N | 736 |
| | STSNLASP | G8→P | 737 |
| | STSNLASR | G8→R | 738 |
| | STSNLASS | G8→S | 739 |
| | STSNLAST | G8→T | 740 |
| | STSNLASV | G8→V | 741 |
| | STSNLASY | G8→Y | 742 |
| Light chain | HQYHRSPPTF | | 357 |
| CDR3 sequence | AQYHRSPPTF | H1→A | 743 |
| | CQYHRSPPTF | H1→C | 744 |
| | DQYHRSPPTF | H1→D | 745 |
| | EQYHRSPPTF | H1→E | 746 |
| | FQYHRSPPTF | H1→F | 747 |
| | IQYHRSPPTF | H1→I | 748 |
| | KQYHRSPPTF | H1→K | 749 |
| | LQYHRSPPTF | H1→L | 750 |
| | NQYHRSPPTF | H1→N | 751 |
| | QQYHRSPPTF | H1→Q | 836 |
| | RQYHRSPPTF | H1→R | 752 |
| | SQYHRSPPTF | H1→S | 753 |
| | TQYHRSPPTF | H1→T | 754 |
| | VQYHRSPPTF | H1→V | 755 |
| | WQYHRSPPTF | H1→W | 756 |
| | YQYHRSPPTF | H1→Y | 757 |
| | HAYHRSPPTF | Q2→A | 758 |
| | HCYHRSPPTF | Q2→C | 759 |
| | HEYHRSPPTF | Q2→E | 760 |
| | HGYHRSPPTF | Q2→G | 761 |
| | HNYHRSPPTF | Q2→N | 762 |
| | HSYHRSPPTF | Q2→S | 763 |
| | HTYHRSPPTF | Q2→T | 764 |
| | HVYHRSPPTF | Q2→V | 765 |
| | HQAHRSPPTF | Y3→A | 766 |
| | HQFHRSPPTF | Y3→F | 767 |
| | HQGHRSPPTF | Y3→G | 768 |
| | HQHHRSPPTF | Y3→H | 769 |
| | HQQHRSPPTF | Y3→Q | 770 |
| | HQWHRSPPTF | Y3→W | 771 |
| | HQYARSPPTF | H4→A | 772 |
| | HQYERSPPTF | H4→E | 773 |
| | HQYGRSPPTF | H4→G | 774 |
| | HQYKRSPPTF | H4→K | 775 |
| | HQYLRSPPTF | H4→L | 776 |
| | HQYNRSPPTF | H4→N | 777 |
| | HQYQRSPPTF | H4→Q | 778 |
| | HQYSRSPPTF | H4→S | 779 |
| | HQYTRSPPTF | H4→T | 780 |
| | HQYVRSPPTF | H4→V | 781 |
| | HQYWRSPPTF | H4→W | 782 |
| | HQYHASPPTF | R5→A | 783 |
| | HQYHCSPPTF | R5→C | 784 |
| | HQYHDSPPTF | R5→D | 785 |
| | HQYHESPPTF | R5→E | 786 |
| | HQYHFSPPTF | R5→F | 787 |
| | HQYHISPPTF | R5→I | 788 |
| | HQYHLSPPTF | R5→L | 789 |
| | HQYHMSPPTF | R5→M | 790 |
| | HQYHNSPPTF | R5→N | 791 |
| | HQYHQSPPTF | R5→Q | 792 |
| | HQYHVSPPTF | R5→V | 793 |
| | HQYHWSPPTF | R5→W | 794 |
| | HQYHYSPPTF | R5→Y | 795 |
| | HQYHRAPPTF | S6→A | 796 |
| | HQYHRCPPTF | S6→C | 797 |
| | HQYHREPPTF | S6→E | 798 |
| | HQYHRFPPTF | S6→F | 799 |
| | HQYHRGPPTF | S6→G | 800 |
| | HQYHRIPPTF | S6→I | 801 |
| | HQYHRMPPTF | S6→M | 802 |
| | HQYHRPPPTF | S6→P | 803 |
| | HQYHRRPPTF | S6→R | 804 |
| | HQYHRTPPTF | S6→T | 805 |
| | HQYHRVPPTF | S6→V | 806 |
| | HQYHRWPPTF | S6→W | 807 |
| | HQYHRYPPTF | S6→Y | 808 |
| | HQYHRSKPTF | P7→K | 809 |
| | HQYHRSWPTF | P7→W | 810 |
| | HQYHRSYPTF | P7→Y | 811 |
| | HQYHRSPDTF | P8→D | 812 |
| | HQYHRSPHTF | P8→H | 813 |
| | HQYHRSPPDF | T9→D | 814 |
| | HQYHRSPPFF | T9→F | 815 |
| | HQYHRSPPGF | T9→G | 816 |
| | HQYHRSPPIF | T9→I | 817 |
| | HQYHRSPPLF | T9→L | 818 |
| | HQYHRSPPMF | T9→M | 819 |
| | HQYHRSPPNF | T9→N | 820 |
| | HQYHRSPPQF | T9→Q | 821 |
| | HQYHRSPPRF | T9→R | 822 |
| | HQYHRSPPSF | T9→S | 823 |
| | HQYHRSPPVF | T9→V | 824 |
| | HQYHRSPPYF | T9→Y | 825 |

TABLE 8
Sequences of Light Chain Affinity Matured P1C4 Clones

| Antibody | | |
|---|---|---|
| Name/ | | |
| Sequence | | SEQ ID |
| Description | Sequence | NO: |
| RY-LC-B12 | EIVLTQSPATLSLSPGERATLSCRASQSVSSRYLHWYQQKPGQAPRLL | 829 |
| FACS3 | IYSTSRLASGIPARFSGSGSGTDFTLTISRLEPEDFAVYYCQQYHRSP | |
| | PTFGQGTKVEIKRTVAAPSVFIFPPSDEQLKSGTASVVCLLNNFYPRE | |
| | AKVQWKVDNALQSGNSQESVTEQDSKDSTYSLSSTLTLSKADYEKHKV | |
| | YACEVTHQGLSSPVTKSFNRGEC | |
| RY-LC-A10 | EIVLTQSPATLSLSPGERATLSCRASQSVSSRYLHWYQQKPGQAPRLL | 830 |
| FACS5 | IYSTSNRASGIPARFSGSGSGTDFTLTISRLEPEDFAVYYCQQYHRSP | |
| | PTFGQGTKVEIKRTVAAPSVFIFPPSDEQLKSGTASVVCLLNNFYPRE | |
| | AKVQWKVDNALQSGNSQESVTEQDSKDSTYSLSSTLTLSKADYEKHKV | |
| | YACEVTHQGLSSPVTKSFNRGEC | |
| RY-HL-LC-C12 | EIVLTQSPATLSLSPGERATLSCRASQSVSSRYLHWYQQKPGQAPRLL | 831 |
| FACS4 | IYSTSRLASGIPARFSGSGSGTDFTLTISRLEPEDFAVYYCHQYHRSP | |
| | PTFGQGTKVEIKRTVAAPSVFIFPPSDEQLKSGTASVVCLLNNFYPRE | |
| | AKVQWKVDNALQSGNSQESVTEQDSKDSTYSLSSTLTLSKADYEKHKV | |
| | YACEVTHQGLSSPVTKSFNRGEC | |
| RY-HL-LC-A12 | EIVLTQSPATLSLSPGERATLSCRASQSVSSRYLHWYQQKPGQAPRLL | 832 |
| FACS4 | IYGTSNLASGIPARFSGSGSGTDFTLTISRLEPEDFAVYYCHQYSRSP | |
| | PTFGQGTKVEIKRTVAAPSVFIFPPSDEQLKSGTASVVCLLNNFYPRE | |
| | AKVQWKVDNALQSGNSQESVTEQDSKDSTYSLSSTLTLSKADYEKHKV | |
| | YACEVTHQGLSSPVTKSFNRGEC | |

TABLE 9
Summary of Amino Acid Changes in Affinity Matured Clones

| Source Sequence | | | |
|---|---|---|---|
| Description | Sequence | Mutation | SEQ ID NO |
| Light chain CDR1 sequence | SSYLH | | 355 |
| | SRYLH | S2→R | 833 |
| Light chain CDR2 sequence | STSNLAS | | 356 |
| | GTSNLAS | S1→G | 834 |
| | STSRLAS | N4→R | 694 |
| | STSNRAS | L5→R | 835 |
| Light chain CDR3 sequence | HQYHRSPPTF | | 357 |
| | QQYHRSPPTF | H1→Q | 836 |
| | HQYSRSPPTF | H4→S | 779 |

1. A method of predicting whether a patient is at risk for developing Fast Progressing Renal Disease (FPRD), the method comprising determining the concentration of one or more endocannabinoids selected from Arachidonoyl Ethanolamide (AE), D-erythro-Sphingosine (DeSph), Di-homo-γ-Linolenoyl Ethanolamide (DhyLE), Docosatetraconoyl Ethanolamide (DE), Linoleoyl Ethanolamide (LE), Palmitoyl Ethanolamide (PE) and Palmitoleoyl Ethanolamine (POEA) in a whole blood, serum or plasma sample from the patient and correlating the concentration of POEA in the sample to the likelihood that the patient will develop FPRD.

2. The method of claim 1, wherein the patient is suffering from diabetes or pre-diabetes.

3. The method of claim 2, wherein the diabetes is severe insulin resistant diabetes (SIRD).

4. The method of any of claims 1-3, wherein the correlating comprises determining that the patient will develop FPRD when the level of POEA in the sample exceeds about 0.1 mg.

5. The method of claim 1, wherein the correlating comprises determine that the patient will develop FPRD when the level of POEA in the sample exceeds about the 90th percentile of FPRD in a population of diabetic patients.

6. The method of claim 2, wherein the correlating comprises determining that the patient will develop FPRD when the level of POEA in the sample exceeds about the 90th percentile of FPRD in a population of patients suffering from pre-diabetes.

7. The method of any preceding claim, wherein the correlating comprises predicting that the patient will develop FPRD in 1-5 years when the level of POEA in the sample exceeds about 0.1 mg.

8. A method of identifying a diabetes patient that will develop FPRD, comprising determining the concentration of palmitoleoyl ethanolamide (POEA) in a whole blood, serum or plasma sample from the patient and determining that that the patient will develop FPRD when the concentration of POEA in the sample exceeds about the 90th percentile of the POEA concentration in a population of diabetes patients.

9. A method of treating a patient that has an increased risk for FPRD, comprising
a. determining the concentration of palmitoylethanolamide (POEA) in a whole blood, serum or plasma sample from the patient,
b. determining that that the patient has an increased risk of developing FPRD when the concentration of POEA in the sample exceeds about the 90th percentile of the POEA concentration in a population of diabetes patients; and
c. treating the patient with an antagonist of cannabinoid 1 (CB1) receptor.

10. A method of treating a patient that has SIRD to avoid progression to FPRD, comprising
a. determining the concentration of palmitoylethanolamide (POEA) in a whole blood, serum or plasma sample from the patient,
b. determining an expected onset of FPRD based upon the concentration of POEA in the sample; and
c. treating the patient with an antagonist of cannabinoid 1 (CB1) receptor.

11. The method of claim 9 or claim 10, wherein the antagonist is an antibody or antigen binding fragment thereof that binds CB1 receptor, comprising:
a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 355, a CDR2 amino acid sequence of SEQ ID NO: 356, and a CDR3 amino acid sequence of SEQ ID NO: 357.

12. The method of claim 9 or claim 10, wherein the antibody or antigen binding fragment comprises,
a. a heavy chain variable region comprising an amino acid sequence at least 95% identical to SEQ ID NO: 340 or 341, and
b. a light chain variable region comprising an amino acid sequence at least 95% identical to SEQ ID NO:337.

13. The method of claim 9 or claim 10, wherein the antibody or antigen binding fragment comprises,
a. a heavy chain amino acid sequence comprising SEQ ID NO: 437, and
b. a light chain amino acid sequence comprising SEQ ID NO:338.

14. The method of claim 10, wherein the antibody or antigen binding fragment has at least one mutation identified in Table 6 of Table 7.

15. The method of claim 9 or claim 10, wherein the antagonist is an antibody or antigen binding fragment thereof, comprising:
a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 694, and a CDR3 amino acid sequence of SEQ ID NO: 836.

16. The method of claim 9 or claim 10, wherein the antagonist is an antibody or antigen binding fragment thereof, comprising:
a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 835, and a CDR3 amino acid sequence of SEQ ID NO: 836.

17. The method of claim 9 or claim 10, wherein the antagonist is an antibody or antigen binding fragment thereof, comprising:
a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 694, and a CDR3 amino acid sequence of SEQ ID NO: 357.

18. The method of claim 9 or claim 10, wherein the antagonist is an antibody or antigen binding fragment thereof, comprising:
a. a heavy chain variable region comprising a CDR1 amino acid sequence of SEQ ID NO: 352, a CDR2 amino acid sequence of SEQ ID NO: 353, and a CDR3 amino acid sequence of SEQ ID NO: 354, and
b. a light chain variable region comprising a CDR1 amino acid sequence of SEQ ID NO: 833, a CDR2 amino acid sequence of SEQ ID NO: 834, and a CDR3 amino acid sequence of SEQ ID NO: 779.

19. The method of claim 15, wherein the antibody or antigen binding fragment thereof comprises a light chain according to SEQ ID NO: 829.

20. The method of claim 16, wherein the antibody or antigen binding fragment thereof comprises a light chain according to SEQ ID NO: 830.

21. The method of claim 17, wherein the antibody or antigen binding fragment thereof comprises a light chain according to SEQ ID NO: 831.

22. The method of claim 18, wherein the antibody or antigen binding fragment thereof comprises a light chain according to SEQ ID NO: 832.

23. The method of any of one claims 19-22, wherein the antibody or antigen binding fragment thereof comprises a heavy chain according to SEQ ID NO: 437.


SEQUENCE LISTING (836 sequences)

SEQ ID NO: 1 - DNA, 351 bp, synthetic construct
  misc_feature 1..351: note = PA13R3-P1C4 Heavy chain variable region
  source 1..351: mol_type = other DNA
gaggtccagctgcagcagtctggggctgagctggtgaggcctggggtctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccaacacagcctatatgcagctcagcagcctaacatctgaggactctgcggtctatttctgtgcaagatcccatggtaactaccttccttactggggccaagggactctggtcactgtctctgca

SEQ ID NO: 2 - protein, 117 aa, synthetic construct
  REGION 1..117: note = PA13R3-P1C4 Heavy chain variable region
EVQLQQSGAELVRPGVSVKISCKASGYEFSYYWMNWVKQRPGQGLEWIGQIYPGDGETKYNGKFKGKATLTADKSSNTAYMQLSSLTSEDSAVYFCARSHGNYLPYWGQGTLVTVSA

SEQ ID NO: 3 - DNA, 993 bp, synthetic construct
  misc_feature 1..993: note = PA13R3-P1C4 Heavy chain constant region
  source 1..993: mol_type = other DNA
gcctccaccaagggcccatcggtcttccccctggcaccctcctccaagagcacctctgggggcacagcggccctgggctgcctggtcaaggactacttccccgaaccggtgacggtgtcatggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 330 aa, synthetic construct [sequence omitted]

SEQ ID NO: 5 - DNA, 324 bp, synthetic construct
  misc_feature 1..324: note = PA13R3-P1C4 Light chain variable region
  source 1..324: mol_type = other DNA
gatattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaa

SEQ ID NO: 6 - protein, 108 aa, synthetic construct
  REGION 1..108: note = PA13R3-P1C4 Light chain variable region
DIVLTQSPAIMSASLGERVTMTCTASSSVSSSYLHWYQQKPGSSPKLWIYSTSNLASGVPARFSGSGSGTSYSLTISSMEAEDAATYYCHQYHRSPPTFGAGTKLELK

SEQ ID NO: 7 - DNA, 324 bp, synthetic construct
  misc_feature 1..324: note = PA13R3-P1C4 Light chain constant region
  source 1..324: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgttaa

SEQ ID NO: 8 - protein, 107 aa, synthetic construct
  REGION 1..107: note = PA13R3-P1C4 Light chain constant region
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSLPVTKSFNRGEC

SEQ ID NO: 9 - DNA, 351 bp, synthetic construct
  misc_feature 1..351: note = 36E12B6C2 Heavy chain variable region
  source 1..351: mol_type = other DNA
caggttcagctgcagcagtctggggctgagctggtgaggcctgggtcctcagtgaagatttcctgcaaggcttctggctatgaattcagttactactggatgaactgggtgaagcagaggcctggacagggtcttcagtggattggacagatttatcctggagatggtgatactaactacaatggaaagttcaagggtaaagccacactgactgcagacaaatcctccagcacagcctacatgcacctcaccagcctaacgtctgaggactctgcggtctatttctgtgcaagatcggggggtaacccctttgctttctggggccaagggactctggtcactgtctctgca

SEQ ID NO: 10 - protein, 117 aa, synthetic construct
  REGION 1..117: note = 36E12B6C2 Heavy chain variable region
QVQLQQSGAELVRPGSSVKISCKASGYEFSYYWMNWVKQRPGQGLQWIGQIYPGDGDTNYNGKFKGKATLTADKSSSTAYMHLTSLTSEDSAVYFCARSGGNPFAFWGQGTLVTVSA

SEQ ID NO: 11 - DNA, 993 bp, synthetic construct; identical to SEQ ID NO: 3
  misc_feature 1..993: note = 36E12B6C2 Heavy chain constant region
  source 1..993: mol_type = other DNA

SEQ ID NO: 12 - protein, 330 aa, synthetic construct [sequence omitted]

SEQ ID NO: 13 - DNA, 321 bp, synthetic construct
  misc_feature 1..321: note = 36E12B6C2 Light chain variable region
  source 1..321: mol_type = other DNA
gatatccagatgacacagacttcatcctccctgtctgcctctctgggagacagagtcaccttcagttgcagggcaagtcaggacattagcaattatttaaactggtatcagcagaaaccagatggaactgttaaactcctgatctactacacatcaagattacactcaggagtcacatcaaggttccgtggcagtgggtctggaacagattattctctcaccattagcaacctggagcaagaagacgttgccacttacttttgccaacagggtcatacgcttccgtggtcgttcggtggaggcaccaagctggaaatcaaa

SEQ ID NO: 14 - protein, 107 aa, synthetic construct
  REGION 1..107: note = 36E12B6C2 Light chain variable region
DIQMTQTSSSLSASLGDRVTFSCRASQDISNYLNWYQQKPDGTVKLLIYYTSRLHSGVTSRFRGSGSGTDYSLTISNLEQEDVATYFCQQGHTLPWSFGGGTKLEIK

SEQ ID NO: 15 - DNA, 324 bp, synthetic construct
  misc_feature 1..324: note = 36E12B6C2 Light chain constant region
  source 1..324: mol_type = other DNA
cgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 16 - protein, 107 aa, synthetic construct [sequence omitted]

SEQ ID NO: 17 - DNA, 351 bp, Mus sp.; identical to SEQ ID NO: 9
  source 1..351: mol_type = other DNA

SEQ ID NO: 18 - protein, 117 aa, Mus sp.; identical to SEQ ID NO: 10

SEQ ID NO: 19 - DNA, 993 bp, Mus sp.
  source 1..993: mol_type = other DNA
gctaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaatga

SEQ ID NO: 20 - protein, 330 aa, Mus sp.
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 21 - DNA, 321 bp, Mus sp.; identical to SEQ ID NO: 13
  source 1..321: mol_type = other DNA

SEQ ID NO: 22 - protein, 107 aa, Mus sp.; identical to SEQ ID NO: 14

SEQ ID NO: 23 - DNA, 324 bp, Mus sp.
  source 1..324: mol_type = other DNA
cgggcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 24 - protein, 107 aa, Mus sp.
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - DNA, 351 bp, synthetic construct
  misc_feature 1..351: note = PA2LR3- P4B5 Heavy chain variable region
  source 1..351: mol_type = other DNA
gaggttcagctgcagcagtctggggctgaactggtgaggcctgggtcctcagtgaagatttcctgcaaggcttctggctatgcattcagttattactggatgaactgggtgaagcagaggcctggacagggtcttgagtggattggacagatttatcctggagatggtgatactaactacagtggaaggttcaagggtaaagccacactgactgcagacaaatcctccagcacagcctacattcagctcagcagcctaacatctgaggactctgcggtctatttctgtgcaagatcgcacggtaactattttccttactggggccaagggactctggtcactgtctctgca

SEQ ID NO: 26 - protein, 117 aa, synthetic construct
  REGION 1..117: note = PA2LR3- P4B5 Heavy chain variable region
EVQLQQSGAELVRPGSSVKISCKASGYAFSYYWMNWVKQRPGQGLEWIGQIYPGDGDTNYSGRFKGKATLTADKSSSTAYIQLSSLTSEDSAVYFCARSHGNYFPYWGQGTLVTVSA

SEQ ID NO: 27 - DNA, 993 bp, synthetic construct; identical to SEQ ID NO: 3
  misc_feature 1..993: note = PA2LR3- P4B5 Heavy chain constant region
  source 1..993: mol_type = other DNA

SEQ ID NO: 28 - protein, 330 aa, synthetic construct [sequence omitted]

SEQ ID NO: 29 - DNA, 324 bp, synthetic construct
  misc_feature 1..324: note = PA2LR3- P4B5 Light chain variable region
  source 1..324: mol_type = other DNA
gacattgttctcaaccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccgctcacgttcggtgctgggaccaaactggaaataaaa

SEQ ID NO: 30 - protein, 108 aa, synthetic construct
  REGION 1..108: note = PA2LR3- P4B5 Light chain variable region
DIVLNQSPAIMSASLGERVTMTCTASSSVSSSYLHWYQQKPGSSPKLWIYSTSNLASGVPARFSGSGSGTSYSLTISSMEAEDAATYYCHQYHRSPLTFGAGTKLEIK

SEQ ID NO: 31 - DNA, 324 bp, synthetic construct; identical to SEQ ID NO: 7
  misc_feature 1..324: note = PA2LR3- P4B5 Light chain constant region
  source 1..324: mol_type = other DNA

SEQ ID NO: 32 - protein, 107 aa, synthetic construct; identical to SEQ ID NO: 8
  REGION 1..107: note = PA2LR3- P4B5 Light chain constant region

SEQ ID NO: 33 - DNA, 351 bp, synthetic construct
  misc_feature 1..351: note = PA2LR3-P2D3 Heavy chain variable region
  source 1..351: mol_type = other DNA
gaggttcagctgcagcagtctggggctgagctggtgaggcctgggtcctcagtgaagatttcctgcaaggcttctggctatgcattcagttactactggatgaactgggtgaagcagaggcctggacagggtcttgagtggattggacagatttatcctggagatggtgatactaactacaatggaaagttcaagggtaaagccacactgactgcagacaaatcctccagtacagcctacatgcagctcagcagcctaacatctgaggactctgcggtctatttctgtgcaagatcgcacggtagctattttgcttactggggccaagggactctggtcactgtctctgca

SEQ ID NO: 34 - protein, 117 aa, synthetic construct
  REGION 1..117: note = PA2LR3-P2D3 Heavy chain variable region
EVQLQQSGAELVRPGSSVKISCKASGYAFSYYWMNWVKQRPGQGLEWIGQIYPGDGDTNYNGKFKGKATLTADKSSSTAYMQLSSLTSEDSAVYFCARSHGSYFAYWGQGTLVTVSA

SEQ ID NO: 35 - DNA, 993 bp, synthetic construct; identical to SEQ ID NO: 3
  misc_feature 1..993: note = PA2LR3-P2D3 Heavy chain constant region
  source 1..993: mol_type = other DNA

SEQ ID NO: 36 - protein, 330 aa, synthetic construct [sequence omitted]

SEQ ID NO: 37 - DNA, 333 bp, synthetic construct
  misc_feature 1..333: note = PA2LR3-P2D3 Light chain variable region
  source 1..333: mol_type = other DNA
gatattgagctggcccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagcggcagtgggtctagggcagacttcaccctcaccattgatcctgtggaggctgatgatgctgcaacctattactgtctacaatatgctagttctcctcctacgttcggtgctgggaccaaactggaaataaaa

SEQ ID NO: 38 - protein, 111 aa, synthetic construct
  REGION 1..111: note = PA2LR3-P2D3 Light chain variable region
DIELAQSPASLAVSLGQRATISCRASESVDSYGNSFMHWYQQKPGQPPKLLIYLASNLESGVPARFSGSGSRADFTLTIDPVEADDAATYYCLQYASSPPTFGAGTKLEIK

SEQ ID NO: 39 - DNA, 324 bp, synthetic construct; identical to SEQ ID NO: 7
  misc_feature 1..324: note = PA2LR3-P2D3 Light chain constant region
  source 1..324: mol_type = other DNA

SEQ ID NO: 40 - protein, 107 aa, synthetic construct; identical to SEQ ID NO: 8
  REGION 1..107: note = PA2LR3-P2D3 Light chain constant region

SEQ ID NO: 41 - DNA, 351 bp, synthetic construct
  misc_feature 1..351: note = PA2LR3-P4B1 Heavy chain variable region
  source 1..351: mol_type = other DNA
gaggtccagcttcagcaatctggggctgagctggtgaggcctgggtcctcagtgaagatttcctgcaaggcttctggctttgcattcagtaactactggatgaactgggtgaagcagaggcctggacagggtcttgagtggattggacagatttatcctggagatggtgatactaacttcaatggaaagttcaagggtagagccatactgactgcagacatatcctccaacacagcctacatgcagctcagcagcctaacatctgaggactctgcggtctatttctgtgcaagatcgcacggtaactattttccttactggggccaagggactctggtcactgtctctgca

SEQ ID NO: 42 - protein, 117 aa, synthetic construct
  REGION 1..117: note = PA2LR3-P4B1 Heavy chain variable region
EVQLQQSGAELVRPGSSVKISCKASGFAFSNYWMNWVKQRPGQGLEWIGQIYPGDGDTNFNGKFKGRAILTADISSNTAYMQLSSLTSEDSAVYFCARSHGNYFPYWGQGTLVTVSA

SEQ ID NO: 43 - DNA, 993 bp, synthetic construct; identical to SEQ ID NO: 3
  misc_feature 1..993: note = PA2LR3-P4B1 Heavy chain constant region
  source 1..993: mol_type = other DNA

SEQ ID NO: 44 - protein, 330 aa, synthetic construct [sequence omitted]

SEQ ID NO: 45 - DNA, 324 bp, synthetic construct
  misc_feature 1..324: note = PA2LR3-P4B1 Light chain variable region
  source 1..324: mol_type = other DNA
caaattgtgttgacacagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccgctcacgttcggtgctgggaccaagctggagctgaaa

SEQ ID NO: 46 - protein, 108 aa, synthetic construct
  REGION 1..108: note = PA2LR3-P4B1 Light chain variable region
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPLTFGAGTKLELK

SEQ ID NO: 47 - DNA, 324 bp, synthetic construct; identical to SEQ ID NO: 7
  misc_feature 1..324: note = PA2LR3-P4B1 Light chain constant region
  source 1..324: mol_type = other DNA

SEQ ID NO: 48 - protein, 107 aa, synthetic construct; identical to SEQ ID NO: 8
  REGION 1..107: note = PA2LR3-P4B1 Light chain constant region

SEQ ID NO: 49 - DNA, 351 bp, synthetic construct
  misc_feature 1..351: note = PA2LR3-P6B12 Heavy chain variable region
  source 1..351: mol_type = other DNA
gaggtccagcttcagcaatctggggctgagctggtgaggcctgggtcctcagtgaagatttcctgcaaggcttctggctatgcattcagttactactggatgaactgggtgaagcagaggcctggacagggtcttgagtggattggacagatttatcctggagatggtgatactaactacaatggaaagttcaagggtaaagccacactgactgcagacaaatcctccagtacagcctacatgcagctcagcagcctaacatctgaggactctgcggtctatttctgtgcaagatcgcacggtaactattttgcttactggggccaagggactctggtcactgtctctgca

SEQ ID NO: 50 - protein, 117 aa, synthetic construct
  REGION 1..117: note = PA2LR3-P6B12 Heavy chain variable region
EVQLQQSGAELVRPGSSVKISCKASGYAFSYYWMNWVKQRPGQGLEWIGQIYPGDGDTNYNGKFKGKATLTADKSSSTAYMQLSSLTSEDSAVYFCARSHGNYFAYWGQGTLVTVSA

SEQ ID NO: 51 - DNA, 993 bp, synthetic construct; identical to SEQ ID NO: 3
  misc_feature 1..993: note = PA2LR3-P6B12 Heavy chain constant region
  source 1..993: mol_type = other DNA

SEQ ID NO: 52 - protein, 330 aa, synthetic construct [sequence omitted]

SEQ ID NO: 53 - DNA, 324 bp, synthetic construct
  misc_feature 1..324: note = PA2LR3-P6B12 Light chain variable region
  source 1..324: mol_type = other DNA
caaattgtactcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttcccccctcgcgttcggtgctgggaccaagctggagctgaaa

SEQ ID NO: 54 - protein, 108 aa, synthetic construct
  REGION 1..108: note = PA2LR3-P6B12 Light chain variable region
QIVLTQSPAIMSASLGERVTMTCTASSSVSSSYLHWYQQKPGSSPKLWIYSTSNLASGVPARFSGSGSGTSYSLTISSMEAEDAATYYCHQYHRSPLAFGAGTKLELK

SEQ ID NO: 55 - DNA, 324 bp, synthetic construct; identical to SEQ ID NO: 7
  misc_feature 1..324: note = PA2LR3-P6B12 Light chain constant region
  source 1..324: mol_type = other DNA

SEQ ID NO: 56 - protein, 107 aa, synthetic construct; identical to SEQ ID NO: 8
  REGION 1..107: note = PA2LR3-P6B12 Light chain constant region

SEQ ID NO: 57 - DNA, 351 bp, synthetic construct
  misc_feature 1..351: note = PA2LR3-P6G7 Heavy chain variable region
  source 1..351: mol_type = other DNA
gaggttcagcttcagcaatctggggctgagctggtgaggcctgggtcctcagtgaagatttcctgcaaggcttctggctttgcattcagtaactactggatgaactgggtgaagcagaggcccggacagggtcttgagtggattggacagatttatcctggagatggtgatactaacttcaatggaaagttcaagggtagagccatactgactgcagacatatcctccaacacagcctacatgcagctcagcagcctaacatctgaggactctgcggtctatttctgtgcaagatcgcacggtaactattttccttactggggccaagggactctggtcactgtctctgca

SEQ ID NO: 58 - protein, 117 aa, synthetic construct; identical to SEQ ID NO: 42
  REGION 1..117: note = PA2LR3-P6G7 Heavy chain variable region

SEQ ID NO: 59 - DNA, 993 bp, synthetic construct; identical to SEQ ID NO: 3
  misc_feature 1..993: note = PA2LR3-P6G7 Heavy chain constant region
  source 1..993: mol_type = other DNA

SEQ ID NO: 60 - protein, 330 aa, synthetic construct [sequence omitted]

SEQ ID NO: 61 - DNA, 324 bp, synthetic construct
  misc_feature 1..324: note = PA2LR3-P6G7 Light chain variable region
  source 1..324: mol_type = other DNA
gatattgtgctaactcagtctccagcaatcatgtccgcatctctaggggaacgggtcaccatgacctgcactgccagctcaagtgtaagttccagttacttacactggtaccagcagaagccaggatcctcccccaaactctggatttatagcacctccaacctggcttctggagtcccagctcgcttcagtggcagtgggtctgggacctcttactctctcacaatcagcagcatggaggctgaagatgctgccacttattactgccaccagcatcatcgttccccacccacgttcggtgctgggaccaagctggagctgaaa

SEQ ID NO: 62 - protein, 108 aa, synthetic construct
  REGION 1..108: note = PA2LR3-P6G7 Light chain variable region
DIVLTQSPAIMSASLGERVTMTCTASSSVSSSYLHWYQQKPGSSPKLWIYSTSNLASGVPARFSGSGSGTSYSLTISSMEAEDAATYYCHQHHRSPPTFGAGTKLELK

SEQ ID NO: 63 - DNA, 324 bp, synthetic construct; identical to SEQ ID NO: 7
  misc_feature 1..324: note = PA2LR3-P6G7 Light chain constant region
  source 1..324: mol_type = other DNA

SEQ ID NO: 64 - protein, 107 aa, synthetic construct; identical to SEQ ID NO: 8
  REGION 1..107: note = PA2LR3-P6G7 Light chain constant region

SEQ ID NO: 65 - DNA, 351 bp, synthetic construct
  misc_feature 1..351: note = PA2LR3-P1G6 Heavy Chain Variable Region
  source 1..351: mol_type = other DNA
gaggtgcagctgcagcagtctggggctgagctggtgaggcctgggtcctcagtgaagatttcctgcaaggcttctggctatgcattcagttactactggatgaactgggtgaagcagaggcctggacagggtcttgagtggattggacagatttatcctggagatggtgatactaactacaatgggaagttcaagggtaaagccacactgactgtagacaaatcctccagtacagcctacatgcagctcagcagcctaacatctgaggactctgcggtctatttctgtgcaagatcgcacggtaactattttgcttactggggccaagggactctggtcactgtctctgca

SEQ ID NO: 66 - protein, 117 aa, synthetic construct
  REGION 1..117: note = PA2LR3-P1G6 Heavy Chain Variable Region
EVQLQQSGAELVRPGSSVKISCKASGYAFSYYWMNWVKQRPGQGLEWIGQIYPGDGDTNYNGKFKGKATLTVDKSSSTAYMQLSSLTSEDSAVYFCARSHGNYFAYWGQGTLVTVSA

SEQ ID NO: 67 - DNA, 993 bp, synthetic construct; identical to SEQ ID NO: 3
  misc_feature 1..993: note = PA2LR3-P1G6 Heavy Chain Constant Region
  source 1..993: mol_type = other DNA

SEQ ID NO: 68 - protein, 204 aa, synthetic construct
  REGION 1..204: note = PA2LR3-P1G6 Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKC

SEQ ID NO: 69 - DNA, 324 bp, synthetic construct
  misc_feature 1..324: note = PA2LR3-P1G6 Light Chain Variable Region
  source 1..324: mol_type = other DNA
gacattgttctctcccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagccgaatggaggctgaagatgctgccacttattactgccagcagtggagtagttacccactcacgttcggtgctgggaccaagctggagctgaaa

SEQ ID NO: 70 - protein, 108 aa, synthetic construct
  REGION 1..108: note = PA2LR3-P1G6 Light Chain Variable Region
DIVLSQSPAIMSASLGERVTMTCTASSSVSSSYLHWYQQKPGSSPKLWIYSTSNLASGVPARFSGSGSGTSYSLTISRMEAEDAATYYCQQWSSYPLTFGAGTKLELK

SEQ ID NO: 71 - DNA, 324 bp, synthetic construct; identical to SEQ ID NO: 7
  misc_feature 1..324: note = PA2LR3-P1G6 Light Chain Constant Region
  source 1..324: mol_type = other DNA

SEQ ID NO: 72 - protein, 107 aa, synthetic construct; identical to SEQ ID NO: 8
  REGION 1..107: note = PA2LR3-P1G6 Light Chain Constant Region

SEQ ID NO: 73 - DNA, 351 bp, synthetic construct
  misc_feature 1..351: note = PA2LR3-P1H4 Heavy Chain Variable Region
  source 1..351: mol_type = other DNA
gaggttcaactgcagcagtctggggctgagctggtgaggcctgggtcctcagtgaagatttcctgcaaggcttctggctatgtattcggtaactactggatgaactgggtgaagcagaggcctggacagggtcttgagtggattggacagatttatcctggagatggtgatactaactacaatgggaagttcaaaggtaaagccacactgactgcagacaaatcctccagcacagcctacatgcagctcagcagcctgacatctgaggactctgcggtctatttctgtgcaagatcgcacggtaactattttccttactggggccaagggactctggtcactgtctctgca

SEQ ID NO: 74 - protein, 117 aa, synthetic construct
  REGION 1..117: note = PA2LR3-P1H4 Heavy Chain Variable Region
EVQLQQSGAELVRPGSSVKISCKASGYVFGNYWMNWVKQRPGQGLEWIGQIYPGDGDTNYNGKFKGKATLTADKSSSTAYMQLSSLTSEDSAVYFCARSHGNYFPYWGQGTLVTVSA

SEQ ID NO: 75 - DNA, 993 bp, synthetic construct; identical to SEQ ID NO: 3
  misc_feature 1..993: note = PA2LR3-P1H4 Heavy Chain Constant Region
  source 1..993: mol_type = other DNA

SEQ ID NO: 76 - protein, 204 aa, synthetic construct; identical to SEQ ID NO: 68
  REGION 1..204: note = PA2LR3-P1H4 Heavy Chain Constant Region

SEQ ID NO: 77 - DNA, 324 bp, synthetic construct
  misc_feature 1..324: note = PA2LR3-P1H4 Light Chain Variable Region
  source 1..324: mol_type = other DNA
caaattgttctcactcagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgtcaacagtggagtagttacccactcacgttcggtgctgggaccaagctggagctgaaa

SEQ ID NO: 78 - protein, 108 aa, synthetic construct
  REGION 1..108: note = PA2LR3-P1H4 Light Chain Variable Region
QIVLTQSPAIMSASLGERVTMTCTASSSVSSSYLHWYQQKPGSSPKLWIYSTSNLASGVPARFSGSGSGTSYSLTISSMEAEDAATYYCQQWSSYPLTFGAGTKLELK

SEQ ID NO: 79 - DNA, 324 bp, synthetic construct; identical to SEQ ID NO: 7
  misc_feature 1..324: note = PA2LR3-P1H4 Light Chain Constant Region
  source 1..324: mol_type = other DNA

SEQ ID NO: 80 - protein, 107 aa, synthetic construct; identical to SEQ ID NO: 8
  REGION 1..107: note = PA2LR3-P1H4 Light Chain Constant Region

SEQ ID NO: 81 - DNA, 351 bp, synthetic construct; identical to SEQ ID NO: 49
  misc_feature 1..351: note = PA2LR3-P2B8 Heavy Chain Variable Region
  source 1..351: mol_type = other DNA

SEQ ID NO: 82 - protein, 117 aa, synthetic construct; identical to SEQ ID NO: 50
  REGION 1..117: note = PA2LR3-P2B8 Heavy Chain Variable Region

SEQ ID NO: 83 - DNA, 993 bp, synthetic construct; identical to SEQ ID NO: 3
  misc_feature 1..993: note = PA2LR3-P2B8 Heavy Chain Constant Region
  source 1..993: mol_type = other DNA

SEQ ID NO: 84 - protein, 204 aa, synthetic construct; identical to SEQ ID NO: 68
  REGION 1..204: note = PA2LR3-P2B8 Heavy Chain Constant Region

SEQ ID NO: 85 - DNA, 324 bp, synthetic construct
  misc_feature 1..324: note = PA2LR3-P2B8 Light Chain Variable Region
  source 1..324: mol_type = other DNA
gatattgtaatgacccagtctccagcaatcatgtctgcatctctaggggaacgggtcaccatgacctgcactgccagctcaagtataagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttcccccctcgcgttcggtgctgggaccaagctggagctgaaa

SEQ ID NO: 86 - protein, 108 aa, synthetic construct
  REGION 1..108: note = PA2LR3-P2B8 Light Chain Variable Region
DIVMTQSPAIMSASLGERVTMTCTASSSISSSYLHWYQQKPGSSPKLWIYSTSNLASGVPARFSGSGSGTSYSLTISSMEAEDAATYYCHQYHRSPLAFGAGTKLELK

SEQ ID NO: 87 - DNA, 324 bp, synthetic construct; identical to SEQ ID NO: 7
  misc_feature 1..324: note = PA2LR3-P2B8 Light Chain Constant Region
  source 1..324: mol_type = other DNA

SEQ ID NO: 88 - protein, 107 aa, synthetic construct; identical to SEQ ID NO: 8
  REGION 1..107: note = PA2LR3-P2B8 Light Chain Constant Region

SEQ ID NO: 89 - DNA, 351 bp, synthetic construct
  misc_feature 1..351: note = PA2LR3-P2E5 Heavy Chain Variable Region
  source 1..351: mol_type = other DNA
gaggttcagctgcagcagtctggggctgagctggtgaggcctgggtcctcagtgaagatttcctgcaaggcttctggctatgcattccgtgactactggatgaactgggtgaagcagaggcctggacagggtcttgagtggattggacagatttatcctggagatggtgctactaactacattggaaagttcaagggtaaagccacactgactgcagacaaatcctccagcacagcctacatgcagctcagcagcctgacatctgaggactctgcggtctatttctgtgcaagatcgcacggtaactattttccttactggggccacgggactctggtcactgtctctgca

SEQ ID NO: 90 - protein, 117 aa, synthetic construct
  REGION 1..117: note = PA2LR3-P2E5 Heavy Chain Variable Region
EVQLQQSGAELVRPGSSVKISCKASGYAFRDYWMNWVKQRPGQGLEWIGQIYPGDGATNYIGKFKGKATLTADKSSSTAYMQLSSLTSEDSAVYFCARSHGNYFPYWGHGTLVTVSA

SEQ ID NO: 91 - DNA, 993 bp, synthetic construct; identical to SEQ ID NO: 3
  misc_feature 1..993: note = PA2LR3-P2E5 Heavy Chain Constant Region
  source 1..993: mol_type = other DNA

SEQ ID NO: 92 - protein, 204 aa, synthetic construct; identical to SEQ ID NO: 68
  REGION 1..204: note = PA2LR3-P2E5 Heavy Chain Constant Region

SEQ ID NO: 93 - DNA, 324 bp, synthetic construct
  misc_feature 1..324: note = PA2LR3-P2E5 Light Chain Variable Region
  source 1..324: mol_type = other DNA
caaattgttctcactcagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttattctctcacaatcagcagcatggaggctgaagatgctgccacttattactgtcaacagtggagtagttacccactcacgttcggtgctgggaccaagctggagctgaaa

SEQ ID NO: 94 - protein, 108 aa, synthetic construct; identical to SEQ ID NO: 78
  REGION 1..108: note = PA2LR3-P2E5 Light Chain Variable Region

SEQ ID NO: 95 - DNA, 324 bp, synthetic construct; identical to SEQ ID NO: 7
  misc_feature 1..324: note = PA2LR3-P2E5 Light Chain Constant Region
  source 1..324: mol_type = other DNA

SEQ ID NO: 96 - protein, 107 aa, synthetic construct; identical to SEQ ID NO: 8
  REGION 1..107: note = PA2LR3-P2E5 Light Chain Constant Region

SEQ ID NO: 97 - DNA, 351 bp, synthetic construct
  misc_feature 1..351: note = PA2LR3-P3A8 Heavy Chain Variable Region
  source 1..351: mol_type = other DNA
gaggtccagcttcagcaatctggggctgagctggtgaggcctgggtcctcagtgaagatttcctgcaaggcttctggctatgcattcagttactactggatgaactgggtgaagcagaggcctggacagggtcttgagtggattggacagatttatcctggagatggtgatactaactacaatggaaaggtcaagggtaaagccacactgactgcagacaaatcctccagtacagcctacatgcagctcagcagcctaacatctgaggactctgcggtctatttctgtgcaagatcgcacggtaactattttgcttactggggccaagggactctggtcactgtctctgca

SEQ ID NO: 98 - protein, 117 aa, synthetic construct
  REGION 1..117: note = PA2LR3-P3A8 Heavy Chain Variable Region
EVQLQQSGAELVRPGSSVKISCKASGYAFSYYWMNWVKQRPGQGLEWIGQIYPGDGDTNYNGKVKGKATLTADKSSSTAYMQLSSLTSEDSAVYFCARSHGNYFAYWGQGTLVTVSA

SEQ ID NO: 99 - DNA, 993 bp, synthetic construct; identical to SEQ ID NO: 3
  misc_feature 1..993: note = PA2LR3-P3A8 Heavy Chain Constant Region
  source 1..993: mol_type = other DNA

SEQ ID NO: 100 - protein, 204 aa, synthetic construct; identical to SEQ ID NO: 68
  REGION 1..204: note = PA2LR3-P3A8 Heavy Chain Constant Region

SEQ ID NO: 101 - DNA, 324 bp, synthetic construct
  misc_feature 1..324: note = PA2LR3-P3A8 Light Chain Variable Region
  source 1..324: mol_type = other DNA
gacattgtgatgacccaatctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccagcagtggagtagtaacccgctcacgttcggtgctgggaccaagctggagctgaaa

SEQ ID NO: 102 - protein, 108 aa, synthetic construct
  REGION 1..108: note = PA2LR3-P3A8 Light Chain Variable Region
DIVMTQSPAIMSASLGERVTMTCTASSSVSSSYLHWYQQKPGSSPKLWIYSTSNLASGVPARFSGSGSGTSYSLTISSMEAEDAATYYCQQWSSNPLTFGAGTKLELK

SEQ ID NO: 103 - DNA, 324 bp, synthetic construct; identical to SEQ ID NO: 7
  misc_feature 1..324: note = PA2LR3-P3A8 Light Chain Constant Region
  source 1..324: mol_type = other DNA

SEQ ID NO: 104 - protein, 107 aa, synthetic construct; identical to SEQ ID NO: 8
  REGION 1..107: note = PA2LR3-P3A8 Light Chain Constant Region

SEQ ID NO: 105 - DNA, 351 bp, synthetic construct
  misc_feature 1..351: note = PA2LR3-P3B10 Heavy Chain Variable Region
  source 1..351: mol_type = other DNA
gaggtgcagctgcagcagtctggggctgagctggtgaggcctgggtcctcagtgaagatttcctgcaaggcttctggctatgcattcagttactactggatgaactgggtgaagcagaggcctggacagggtcttgagtggattggacagatttatcctggagatggtgatactaactacaatggaaagttcaagggtaaagccacactgactgcagacaaatcctccagtacagcctacatgcagctcagcagcctaacatctgaggactctgcggtctatttctgtgcaagatcgcacggtaactattttccttactggggccaagggactctggtcactgtctctgca

SEQ ID NO: 106 - protein, 117 aa, synthetic construct
  REGION 1..117: note = PA2LR3-P3B10 Heavy Chain Variable Region
EVQLQQSGAELVRPGSSVKISCKASGYAFSYYWMNWVKQRPGQGLEWIGQIYPGDGDTNYNGKFKGKATLTADKSSSTAYMQLSSLTSEDSAVYFCARSHGNYFPYWGQGTLVTVSA

SEQ ID NO: 107 - DNA, 993 bp, synthetic construct; identical to SEQ ID NO: 3
  misc_feature 1..993: note = PA2LR3-P3B10 Heavy Chain Constant Region
  source 1..993: mol_type = other DNA

SEQ ID NO: 108 - protein, 204 aa, synthetic construct; identical to SEQ ID NO: 68
  REGION 1..204: note = PA2LR3-P3B10 Heavy Chain Constant Region

SEQ ID NO: 109 - DNA, 324 bp, synthetic construct
  misc_feature 1..324: note = PA2LR3-P3B10 Light Chain Variable Region
  source 1..324: mol_type = other DNA
gacattgtgataactcagtctccagcaatcgtgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttattctctcacaatcagcagcatggaggctgaagatgccgccacttattactgtcaacagtggagtagttacccactcacgttcggtgctgggaccaagctggaaatcaaa

SEQ ID NO: 110 - protein, 108 aa, synthetic construct
  REGION 1..108: note = PA2LR3-P3B10 Light Chain Variable Region
DIVITQSPAIVSASLGERVTMTCTASSSVSSSYLHWYQQKPGSSPKLWIYSTSNLASGVPARFSGSGSGTSYSLTISSMEAEDAATYYCQQWSSYPLTFGAGTKLEIK

SEQ ID NO: 111 - DNA, 324 bp, synthetic construct; identical to SEQ ID NO: 7
  misc_feature 1..324: note = PA2LR3-P3B10 Light Chain Constant Region
  source 1..324: mol_type = other DNA

SEQ ID NO: 112 - protein, 107 aa, synthetic construct; identical to SEQ ID NO: 8
  REGION 1..107: note = PA2LR3-P3B10 Light Chain Constant Region

SEQ ID NO: 113 - DNA, 351 bp, synthetic construct
  misc_feature 1..351: note = PA2LR3-P3B8 Heavy Chain Variable Region
  source 1..351: mol_type = other DNA
gaggtccagctgcagcagtctggggctgagctggtgaggcctgggtcctcagtgaagatttcctgcaaggcttctggctttgcattcagtaactactggatgaactgggtgaagcagaggcccggacagggtcttgagtggattggacagatttatcctggagatggtgatactaacttcaatggaaagttcaagggtagagccatactgactgcagacatatcctccaacacagcctacatgcagctcagcagcctaacatctgaggactctgcggtctatttctgtgcaagatcgcacggtaactattttccttactggggccaagggactctggtcactgtctctgca

SEQ ID NO: 114 - protein, 117 aa, synthetic construct; identical to SEQ ID NO: 42
  REGION 1..117: note = PA2LR3-P3B8 Heavy Chain Variable Region

SEQ ID NO: 115 - DNA, 993 bp, synthetic construct; identical to SEQ ID NO: 3
  misc_feature 1..993: note = PA2LR3-P3B8 Heavy Chain Constant Region
  source 1..993: mol_type = other DNA

SEQ ID NO: 116 - protein, 204 aa, synthetic construct; identical to SEQ ID NO: 68
  REGION 1..204: note = PA2LR3-P3B8 Heavy Chain Constant Region

SEQ ID NO: 117 - DNA, 325 bp, synthetic construct
  misc_feature 1..325: note = PA2LR3-P3B8 Light Chain Variable Region
  source 1..325: mol_type = other DNA
gatattgtgatgacacagtctccagcaatcatgtctgcatctctaggggaacgggtcaccatgacctgcactgccagctcaagtgtaagttccagttacttgcactggtaccagcagaagccaggatcctcccccaaactctggatttatggcacatccaacctggcttctggagtccctgttcgcttcagtggcagtggatctgggacctcttattctctcacaatcagcagcatggaggctgaagatgctgccacttattactgtcaacagtggagtagttacccactcacgttcggtgctgggaccaagctggagctgaaac

SEQ ID NO: 118 - protein, 108 aa, synthetic construct
  REGION 1..108: note = PA2LR3-P3B8 Light Chain Variable Region
DIVMTQSPAIMSASLGERVTMTCTASSSVSSSYLHWYQQKPGSSPKLWIYGTSNLASGVPVRFSGSGSGTSYSLTISSMEAEDAATYYCQQWSSYPLTFGAGTKLELK

SEQ ID NO: 119 - DNA, 324 bp, synthetic construct; identical to SEQ ID NO: 7
  misc_feature 1..324: note = PA2LR3-P3B8 Light Chain Constant Region
  source 1..324: mol_type = other DNA

SEQ ID NO: 120 - protein, 107 aa, synthetic construct; identical to SEQ ID NO: 8
  REGION 1..107: note = PA2LR3-P3B8 Light Chain Constant Region

SEQ ID NO: 121 - DNA, 351 bp, synthetic construct
  misc_feature 1..351: note = PA2LR3-P3F8 Heavy Chain Variable Region
  source 1..351: mol_type = other DNA
gaggttcagcttcagcagtctggggctgagctggtgaggcctgggtcctcagtgaagatttcctgcaaggcttctggctatgcattcagtaactactggatgaactgggtgaagcagaggcctggacagggtcttgagtggattggacagatttatcctggagatggtgatactaactacaatggaaatttcaagggtaaagccacactgactgcagacaaatcctccagcacagcctacatgcagctcagcagcctaacatctgaggactctgcggtctatttctgtgcaagatcgcacggtaactattttccttactggggccaagggactctggtcactgtctctgca

SEQ ID NO: 122 - protein, 117 aa, synthetic construct
  REGION 1..117: note = PA2LR3-P3F8 Heavy Chain Variable Region
EVQLQQSGAELVRPGSSVKISCKASGYAFSNYWMNWVKQRPGQGLEWIGQIYPGDGDTNYNGNFKGKATLTADKSSSTAYMQLSSLTSEDSAVYFCARSHGNYFPYWGQGTLVTVSA

SEQ ID NO: 123 - DNA, 993 bp, synthetic construct; identical to SEQ ID NO: 3
  misc_feature 1..993: note = PA2LR3-P3F8 Heavy Chain Constant Region
  source 1..993: mol_type = other DNA

SEQ ID NO: 124 - protein, 204 aa, synthetic construct; identical to SEQ ID NO: 68
  REGION 1..204: note = PA2LR3-P3F8 Heavy Chain Constant Region

SEQ ID NO: 125 - DNA, 324 bp, synthetic construct
  misc_feature 1..324: note = PA2LR3-P3F8 Light Chain Variable Region
  source 1..324: mol_type = other DNA
gatattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccagcagtggagtagttacccactcacgttcggtgctgggaccaagctggagttgaaa

SEQ ID NO: 126 - protein, 108 aa, synthetic construct
  REGION 1..108: note = PA2LR3-P3F8 Light Chain Variable Region
DIVLTQSPAIMSASLGERVTMTCTASSSVSSSYLHWYQQKPGSSPKLWIYSTSNLASGVPARFSGSGSGTSYSLTISSMEAEDAATYYCQQWSSYPLTFGAGTKLELK

SEQ ID NO: 127 - DNA, 324 bp, synthetic construct; identical to SEQ ID NO: 7
  misc_feature 1..324: note = PA2LR3-P3F8 Light Chain Constant Region
  source 1..324: mol_type = other DNA

SEQ ID NO: 128 - protein, 107 aa, synthetic construct; identical to SEQ ID NO: 8
  REGION 1..107: note = PA2LR3-P3F8 Light Chain Constant Region

SEQ ID NO: 129 - DNA, 351 bp, synthetic construct; identical to SEQ ID NO: 105
  misc_feature 1..351: note = PA2LR3-P4C6 Heavy Chain Variable region
  source 1..351: mol_type = other DNA

SEQ ID NO: 130 - protein, 117 aa, synthetic construct; identical to SEQ ID NO: 106
  REGION 1..117: note = PA2LR3-P4C6 Heavy Chain Variable region

SEQ ID NO: 131 - DNA, 993 bp, synthetic construct; identical to SEQ ID NO: 3
  misc_feature 1..993: note = PA2LR3-P4C6 Heavy Chain Constant region
  source 1..993: mol_type = other DNA

SEQ ID NO: 132 - protein, 204 aa, synthetic construct; identical to SEQ ID NO: 68
  REGION 1..204: note = PA2LR3-P4C6 Heavy Chain Constant region

SEQ ID NO: 133 - DNA, 324 bp, synthetic construct; identical to SEQ ID NO: 109
  misc_feature 1..324: note = PA2LR3-P4C6 Light Chain Variable region
  source 1..324: mol_type = other DNA

SEQ ID NO: 134 - protein, 108 aa, synthetic construct; identical to SEQ ID NO: 110
  REGION 1..108: note = PA2LR3-P4C6 Light Chain Variable region

SEQ ID NO: 135 - DNA, 324 bp, synthetic construct; identical to SEQ ID NO: 7
  misc_feature 1..324: note = PA2LR3-P4C6 Light Chain Constant region
  source 1..324: mol_type = other DNA

SEQ ID NO: 136 - protein, 107 aa, synthetic construct; identical to SEQ ID NO: 8
  REGION 1..107: note = PA2LR3-P4C6 Light Chain Constant region

SEQ ID NO: 137 - DNA, 351 bp, synthetic construct
  misc_feature 1..351: note = PA2LR3-P4G10 Heavy Chain Variable region
  source 1..351: mol_type = other DNA
gaggttcagctgcagcagtttggggctgagctggtgaggcctgggtcctcagtgaagatttcctgcaaggcttctggctatgcattcagttactactggatgaactgggtgaagcagaggcctggacagggtcttgagtggattggacagatttatcctggagatggtgatactaactacaatggaaagttcaagggtaaagccacactgactgcagacaaatcctccagtacagcctacatgcagctcagcagcctaacatctgaggactctgcggtctatttctgtgcaagatcgcacggtaactattttgcttactggggccaagggactctggtcactgtctctgca

SEQ ID NO: 138 - protein, 117 aa, synthetic construct
  REGION 1..117: note = PA2LR3-P4G10 Heavy Chain Variable region
EVQLQQFGAELVRPGSSVKISCKASGYAFSYYWMNWVKQRPGQGLEWIGQIYPGDGDTNYNGKFKGKATLTADKSSSTAYMQLSSLTSEDSAVYFCARSHGNYFAYWGQGTLVTVSA

SEQ ID NO: 139 - DNA, 993 bp, synthetic construct; identical to SEQ ID NO: 3
  misc_feature 1..993: note = PA2LR3-P4G10 Heavy Chain Constant region
  source 1..993: mol_type = other DNA

SEQ ID NO: 140 - protein, 204 aa, synthetic construct; identical to SEQ ID NO: 68
  REGION 1..204: note = PA2LR3-P4G10 Heavy Chain Constant region

SEQ ID NO: 141 - DNA, 324 bp, synthetic construct
  misc_feature 1..324: note = PA2LR3-P4G10 Light Chain Variable region
  source 1..324: mol_type = other DNA
gacattgttctcacccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccagcagtggagtagtaacccgctcacgttcggtgctgggaccaagctggagctgaaa

SEQ ID NO: 142 - protein, 108 aa, synthetic construct
  REGION 1..108: note = PA2LR3-P4G10 Light Chain Variable region
DIVLTQSPAIMSASLGERVTMTCTASSSVSSSYLHWYQQKPGSSPKLWIYSTSNLASGVPARFSGSGSGTSYSLTISSMEAEDAATYYCQQWSSNPLTFGAGTKLELK

SEQ ID NO: 143 - DNA, 324 bp, synthetic construct; identical to SEQ ID NO: 7
  misc_feature 1..324: note = PA2LR3-P4G10 Light Chain Constant region
  source 1..324: mol_type = other DNA

SEQ ID NO: 144 - protein, 107 aa, synthetic construct; identical to SEQ ID NO: 8
  REGION 1..107: note = PA2LR3-P4G10 Light Chain Constant region

SEQ ID NO: 145 - DNA, 351 bp, synthetic construct
  misc_feature 1..351: note = PA2LR3-P5E7 Heavy Chain Variable region
  source 1..351: mol_type = other DNA
gaggtccagctccagcagtctggggctgagctggtgaggcctgggtcctcagtgaagatttcctgcaaggcttctggctatgcattcagtaactactggatgaactgggtgaagcagaggcccggacagggtcttgagtggattggacagatttatcctggagatggtgatactaacttcaatggaaagttcaagggtagagccatactgactgcagacatatcctccaacacagcctacatgcagctcagcagcctaacatctgaggactctgcggtctatttctgtgcaagatcgcacggtaactattttccttactggggccaagggactctggtcactgtctctgca

SEQ ID NO: 146 - protein, 117 aa, synthetic construct
  REGION 1..117: note = PA2LR3-P5E7 Heavy Chain Variable region
EVQLQQSGAELVRPGSSVKISCKASGYAFSNYWMNWVKQRPGQGLEWIGQIYPGDGDTNFNGKFKGRAILTADISSNTAYMQLSSLTSEDSAVYFCARSHGNYFPYWGQGTLVTVSA

SEQ ID NO: 147 - DNA, 993 bp, synthetic construct; identical to SEQ ID NO: 3
  misc_feature 1..993: note = PA2LR3-P5E7 Heavy Chain Constant region
  source 1..993: mol_type = other DNA

SEQ ID NO: 148 - protein, 204 aa, synthetic construct; identical to SEQ ID NO: 68
  REGION 1..204: note = PA2LR3-P5E7 Heavy Chain Constant region

SEQ ID NO: 149 - DNA, 324 bp, synthetic construct
  misc_feature 1..324: note = PA2LR3-P5E7 Light Chain Variable region
  source 1..324: mol_type = other DNA
gacattgtgctcactcagtctccaaccaccatggctgcatctcccggggagaagatcactatcacctgcagtgccagctcaagtataagttccaattacttgcattggtatcagcagaagccaggattctcccctaaactcttgatttataggacatccactctggcttctggagtcccagctcgcttcagtggcagtgggtctgggacctcttactctctcacaatcagcagcatggaggctgaagatgctgccacttattactgccaccagtatcatcgttccccacacacgttcggctcggggaccaaactggaaatcaaa

SEQ ID NO: 150 - protein, 108 aa, synthetic construct
  REGION 1..108: note = PA2LR3-P5E7 Light Chain Variable region
DIVLTQSPTTMAASPGEKITITCSASSSISSNYLHWYQQKPGFSPKLLIYRTSTLASGVPARFSGSGSGTSYSLTISSMEAEDAATYYCHQYHRSPHTFGSGTKLEIK

SEQ ID NO: 151 - DNA, 324 bp, synthetic construct; identical to SEQ ID NO: 7
  misc_feature 1..324: note = PA2LR3-P5E7 Light Chain Constant region
  source 1..324: mol_type = other DNA

SEQ ID NO: 152 - protein, 107 aa, synthetic construct; identical to SEQ ID NO: 8
  REGION 1..107: note = PA2LR3-P5E7 Light Chain Constant region

SEQ ID NO: 153 - DNA, 351 bp, synthetic construct
  misc_feature 1..351: note = PA2LR3-P6D7 Heavy Chain Variable region
  source 1..351: mol_type = other DNA
gaggtgaagctggtggagtctgggggaggtttagtgcagcctggagggtccctgaaactctcctgtgcagcctctggattcactttcagtagctataccatgtcttgggttcgccagactccagagaagaggctggagtgggtcgcatacattagtaatggtggtggtagcacctactatccagacactgtaaagggccgattcaccatctccagagacaatgccaagaacaccctgtacctgcaaatgagcagtctgaagtctgaggacacggccatgtattactgtgcaagacatcgagatggtaactacgactactggggccaaggcaccacggtcaccgtctcctca

SEQ ID NO: 154 - protein, 117 aa, synthetic construct
  REGION 1..117: note = PA2LR3-P6D7 Heavy Chain Variable region
EVKLVESGGGLVQPGGSLKLSCAASGFTFSSYTMSWVRQTPEKRLEWVAYISNGGGSTYYPDTVKGRFTISRDNAKNTLYLQMSSLKSEDTAMYYCARHRDGNYDYWGQGTTVTVSS

SEQ ID NO: 155 - DNA, 993 bp, synthetic construct; identical to SEQ ID NO: 3
  misc_feature 1..993: note = PA2LR3-P6D7 Heavy Chain Constant region
  source 1..993: mol_type = other DNA

SEQ ID NO: 156 - protein, 204 aa, synthetic construct; identical to SEQ ID NO: 68
  REGION 1..204: note = PA2LR3-P6D7 Heavy Chain Constant region

SEQ ID NO: 157 - DNA, 336 bp, synthetic construct
  misc_feature 1..336: note = PA2LR3-P6D7 Light Chain Variable region
  source 1..336: mol_type = other DNA
gatattgtgataactcaggatgaactctccaaccctgtcacttctggagaatcagtttccatctcctgcaggtctagtaagagtctcctatataaggatgggaagacatacttgaattggtttctgcagagaccaggacaatctcctcagctcctgatctatttgatgtccacccgtgcatcaggagtctcagaccggtttagtggcagtgggtcaggaacagatttcaccctggaaatcagtaaagtgaaggctgaggatgtgggtgtgtattactgtcaacaacttgtagagtatccgctcacgttcggtgctgggacaaagttggaaataaaa

SEQ ID NO: 158 - protein, 112 aa, synthetic construct
  REGION 1..112: note = PA2LR3-P6D7 Light Chain Variable region
DIVITQDELSNPVTSGESVSISCRSSKSLLYKDGKTYLNWFLQRPGQSPQLLIYLMSTRASGVSDRFSGSGSGTDFTLEISKVKAEDVGVYYCQQLVEYPLTFGAGTKLEIK

SEQ ID NO: 159 - DNA, 324 bp, synthetic construct; identical to SEQ ID NO: 7
  misc_feature 1..324: note = PA2LR3-P6D7 Light Chain Constant region
  source 1..324: mol_type = other DNA

SEQ ID NO: 160 - protein, 107 aa, synthetic construct; identical to SEQ ID NO: 8
  REGION 1..107: note = PA2LR3-P6D7 Light Chain Constant region

SEQ ID NO: 161 - DNA, 351 bp, synthetic construct
  misc_feature 1..351: note = PA2LR3-P1A7 Heavy Chain Variable region
  source 1..351: mol_type = other DNA
ggggttcaactgcagcagtctggggctgagctggtgaggcctgggtcctcagtgaagatttcctgcaaggcttctggctttgcattcagttactactggatgaactgggtgaagcagaggcccggacagggtcttgagtggattggacagatttatcctggagatggtgatactaacttcaatggaaagttcaagggtagagccatactgactgcagacatatcctccaacacagcctacatgcagctcagcagcctaacatctgaggactctgcggtctatttctgtgcaagatcgcacggtaactattttccttactggggccgagggactctggtcactgtctctgca

SEQ ID NO: 162 - protein, 117 aa, synthetic construct
  REGION 1..117: note = PA2LR3-P1A7 Heavy Chain Variable region
GVQLQQSGAELVRPGSSVKISCKASGFAFSYYWMNWVKQRPGQGLEWIGQIYPGDGDTNFNGKFKGRAILTADISSNTAYMQLSSLTSEDSAVYFCARSHGNYFPYWGRGTLVTVSA

SEQ ID NO: 163 - DNA, 993 bp, synthetic construct; identical to SEQ ID NO: 3
  misc_feature 1..993: note = PA2LR3-P1A7 Heavy Chain Constant region
  source 1..993: mol_type = other DNA

SEQ ID NO: 164 - protein, 204 aa, synthetic construct; identical to SEQ ID NO: 68
  REGION 1..204: note = PA2LR3-P1A7 Heavy Chain Constant region

SEQ ID NO: 165 - DNA, 324 bp, synthetic construct
  misc_feature 1..324: note = PA2LR3-P1A7 Light Chain Variable region
  source 1..324: mol_type = other DNA
gatattgtgctaactcagtctccagcaatcatgtctgcatctctaggggaacgggtcaccatgacctgcactgccagctcaaatgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggtgctgggaccaagctggagctgaaa

SEQ ID NO: 166 - protein, 108 aa, synthetic construct
  REGION 1..108: note = PA2LR3-P1A7 Light Chain Variable region
DIVLTQSPAIMSASLGERVTMTCTASSNVSSSYLHWYQQKPGSSPKLWIYSTSNLASGVPARFSGSGSGTSYSLTISSMEAEDAATYYCHQYHRSPPTFGAGTKLELK

SEQ ID NO: 167 - DNA, 324 bp, synthetic construct; identical to SEQ ID NO: 7
  misc_feature 1..324: note = PA2LR3-P1A7 Light Chain Constant region
  source 1..324: mol_type = other DNA

SEQ ID NO: 168 - protein, 107 aa, synthetic construct; identical to SEQ ID NO: 8
  REGION 1..107: note = PA2LR3-P1A7 Light Chain Constant region

SEQ ID NO: 169 - DNA, 363 bp, synthetic construct
  misc_feature 1..363: note = PA2R3-P1F1 Heavy Chain Variable region
  source 1..363: mol_type = other DNA
gaggtccagctgcagcagtctggagctgagctgatgaagcctggggcctcagtgaagatatcctgcaaggctactggctacacattcagtagctactggatagagtgggtaaagcagaggcctggacatggccttgagtggattggagagattttacctggaagtggtagtactaactacaatgagaagttcaagggcaaggccacattcactgcagatacatcctccaacacagcctatatgcaactcagcagcctgacatctgaggactctgccgtctattactgtgcaagaggggcatggttactatcctactggtacttcgatgtctggggcgcggggacctcagtcaccgtctcctca

SEQ ID NO: 170 - protein, 121 aa, synthetic construct
  REGION 1..121: note = PA2R3-P1F1 Heavy Chain Variable region
EVQLQQSGAELMKPGASVKISCKATGYTFSSYWIEWVKQRPGHGLEWIGEILPGSGSTNYNEKFKGKATFTADTSSNTAYMQLSSLTSEDSAVYYCARGAWLLSYWYFDVWGAGTSVTVSS

SEQ ID NO: 171 - DNA, 993 bp, synthetic construct; identical to SEQ ID NO: 3
  misc_feature 1..993: note = PA2R3-P1F1 Heavy Chain Constant region
  source 1..993: mol_type = other DNA

SEQ ID NO: 172 - protein, 204 aa, synthetic construct; identical to SEQ ID NO: 68
  REGION 1..204: note = PA2R3-P1F1 Heavy Chain Constant region

SEQ ID NO: 173 - DNA, 321 bp, synthetic construct
  misc_feature 1..321: note = PA2R3-P1F1 Light Chain Variable region
  source 1..321: mol_type = other DNA
gacattcagatgatgcagtctacatcctccctgtctgcctctctgggagacagagtcaccatcagttgcagtgcaagtcagggcattagcaattatttaaactggtatcagcagaaaccagatggaactgttaaactcctgatctattacacatcaagtttacactcaggagtcccatcaaggttcagtggcagtggatcaggcacacagttttctctgaagatcaacagcctgcagcctgaagattttgggagttattactgtcaacatcattatggtactccgtacacgttcggaggggggaccaagctggagctgaaa

SEQ ID NO: 174 - protein, 107 aa, synthetic construct
  REGION 1..107: note = PA2R3-P1F1 Light Chain Variable region
DIQMMQSTSSLSASLGDRVTISCSASQGISNYLNWYQQKPDGTVKLLIYYTSSLHSGVPSRFSGSGSGTQFSLKINSLQPEDFGSYYCQHHYGTPYTFGGGTKLELK

SEQ ID NO: 175 - DNA, 324 bp, synthetic construct; identical to SEQ ID NO: 7
  misc_feature 1..324: note = PA2R3-P1F1 Light Chain Constant region
  source 1..324: mol_type = other DNA

SEQ ID NO: 176 - protein, 107 aa, synthetic construct; identical to SEQ ID NO: 8
  REGION 1..107: note = PA2R3-P1F1 Light Chain Constant region

SEQ ID NO: 177 - DNA, 351 bp, synthetic construct
  misc_feature 1..351: note = PA13R3P3-A7 Heavy Chain Variable region
  source 1..351: mol_type = other DNA
gaggtccagctccagcaatctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaactaccttccttactggggccaagggactctggtcactgtctctgca

SEQ ID NO: 178 - protein, 117 aa, synthetic construct
  REGION 1..117: note = PA13R3P3-A7 Heavy Chain Variable region
EVQLQQSGAELLRPGSSVKISCKASGYEFSYYWMNWVKQRPGQGLEWIGQIYPGDGETKYNGKFKGKATLTADKSSSTAYMQLSSLTSEDSAVYFCARSHGNYLPYWGQGTLVTVSA

SEQ ID NO: 179 - DNA, 993 bp, synthetic construct; identical to SEQ ID NO: 3
  misc_feature 1..993: note = PA13R3P3-A7 Heavy Chain Constant region
  source 1..993: mol_type = other DNA

SEQ ID NO: 180 - protein, 204 aa, synthetic construct; identical to SEQ ID NO: 68
  REGION 1..204: note = PA13R3P3-A7 Heavy Chain Constant region

SEQ ID NO: 181 - DNA, 324 bp, synthetic construct
  misc_feature 1..324: note = PA13R3P3-A7 Light Chain Variable region
  source 1..324: mol_type = other DNA
gatattgttctcaaccagtctccagcaatcatgtctgcatctccaggggagaaggtcaccataacctgcactgccagctcaagtgtaagttccagttacttacactggtaccagcagaagccaggatcctcccccaaactctggatttatagcacatccaacctggcttctggagtccctgctcgcttcagtggcagtgggtctgggacctcttactctctcacaatcagcagtatggaggctgaagatgctgccacttattactgccagcagtatcatacttacccacccacgttcggctcggggacaaagttggaaataaaa

SEQ ID NO: 182 - protein, 108 aa, synthetic construct
  REGION 1..108: note = PA13R3P3-A7 Light Chain Variable region
DIVLNQSPAIMSASPGEKVTITCTASSSVSSSYLHWYQQKPGSSPKLWIYSTSNLASGVPARFSGSGSGTSYSLTISSMEAEDAATYYCQQYHTYPPTFGSGTKLEIK

SEQ ID NO: 183 - DNA, 324 bp, synthetic construct
  misc_feature 1..324: note = PA13R3P3-A7 Light Chain Constant region
  source 1..324: mol_type = other DNA
cgaactgtggctgcaccatctgtcttcatcttcccgccatccgatgagcagttgaaatctggaactgcctctgttgtgtgcctgctgaataacttctatcccagagaggccaaagtacagtggaaggtggataacgccccccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagcttgcccgtcacaaagagcttcaacaggggagagtgttaa

SEQ ID NO: 184 - protein, 107 aa, synthetic construct
  REGION 1..107: note = PA13R3P3-A7 Light Chain Constant region
RTVAAPSVFIFPPSDEQLKSGTASVVCLLNNFYPREAKVQWKVDNAPQSGNSQESVTEQDSKDSTYSLSSTLTLSKADYEKHKVYACEVTHQGLSLPVTKSFNRGEC

SEQ ID NO: 185 - DNA, 351 bp, synthetic construct
  misc_feature 1..351: note = PA13R3P3-C3 Heavy Chain Variable region
  source 1..351: mol_type = other DNA
gaggtccagctccagcaatctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagttcagcagcctaacatctgaggactctgcggtctatttctgtgcaagatcccatggtaaccaccttgcttactggggccaagggactctggtcactgtctctgca

SEQ ID NO: 186 - protein, 117 aa, synthetic construct
  REGION 1..117: note = PA13R3P3-C3 Heavy Chain Variable region
EVQLQQSGAELLRPGSSVKISCKASGYEFSYYWMNWVKQRPGQGLEWIGQIYPGDGETKYNGKFKGKATLTADKSSSTAYMQFSSLTSEDSAVYFCARSHGNHLAYWGQGTLVTVSA

SEQ ID NO: 187 - DNA, 993 bp, synthetic construct; identical to SEQ ID NO: 3
  misc_feature 1..993: note = PA13R3P3-C3 Heavy Chain Constant region
  source 1..993: mol_type = other DNA

SEQ ID NO: 188 - protein, 204 aa, synthetic construct; identical to SEQ ID NO: 68
  REGION 1..204: note = PA13R3P3-C3 Heavy Chain Constant region

SEQ ID NO: 189 - DNA, 324 bp, synthetic construct
  misc_feature 1..324: note = PA13R3P3-C3 Light Chain Variable region
  source 1..324: mol_type = other DNA
gatattgttctctcccagtctccagcaatcatgtctgcatctctaggggaacgggtcaccatgacctgcactgccagctcaagtgtaagctccagttacttacactggtaccagcagaagccaggatcctcccccaaactctgggtttatagcacatccaacctggcttctggagtcccagctcgcttcagcggcagtgggtctgggacctcttactctctcacaatcagcagcatggaggctgaagatgctgccacttattactgccaccagtatcatcgttccccacccacgttcggaggggggaccaaactggaaatcaaa

SEQ ID NO: 190 - protein, 108 aa, synthetic construct
  REGION 1..108: note = PA13R3P3-C3 Light Chain Variable region
DIVLSQSPAIMSASLGERVTMTCTASSSVSSSYLHWYQQKPGSSPKLWVYSTSNLASGVPARFSGSGSGTSYSLTISSMEAEDAATYYCHQYHRSPPTFGGGTKLEIK

SEQ ID NO: 191 - DNA, 324 bp, synthetic construct; identical to SEQ ID NO: 7
  misc_feature 1..324: note = PA13R3P3-C3 Light Chain Constant region
  source 1..324: mol_type = other DNA

SEQ ID NO: 192 - protein, 107 aa, synthetic construct; identical to SEQ ID NO: 8
  REGION 1..107: note = PA13R3P3-C3 Light Chain Constant region

SEQ ID NO: 193 - DNA, 351 bp, synthetic construct
  misc_feature 1..351: note = PA13R3P3-D10 Heavy Chain Variable region
  source 1..351: mol_type = other DNA
gaggtccagctccagcaatctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccaacacagcctatatgcagctcagcagcctaacatctgaggactctgcggtctatttctgtacaagatcccatggtaactaccttccttactggggccaagggactctggtcactgtctctgca

SEQ ID NO: 194 - protein, 117 aa, synthetic construct
  REGION 1..117: note = PA13R3P3-D10 Heavy Chain Variable region
EVQLQQSGAELLRPGSSVKISCKASGYEFSYYWMNWVKQRPGQGLEWIGQIYPGDGETKYNGKFKGKATLTADKSSNTAYMQLSSLTSEDSAVYFCTRSHGNYLPYWGQGTLVTVSA

SEQ ID NO: 195 - DNA, 993 bp, synthetic construct; identical to SEQ ID NO: 3
  misc_feature 1..993: note = PA13R3P3-D10 Heavy Chain Constant region
  source 1..993: mol_type = other DNA

SEQ ID NO: 196 - protein, 204 aa, synthetic construct; identical to SEQ ID NO: 68
  REGION 1..204: note = PA13R3P3-D10 Heavy Chain Constant region

SEQ ID NO: 197 - DNA, 324 bp, synthetic construct
  misc_feature 1..324: note = PA13R3P3-D10 Light Chain Variable region
  source 1..324: mol_type = other DNA
gacattgttctctcccagtctccagcaatcatgtctgcatctccaggggaacgggtcaccatgacctgcaccgccagctcaagtgtaagttccagttacttacactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggaggggggaccaagctggagctgaaa

SEQ ID NO: 198 - protein, 108 aa, synthetic construct
  REGION 1..108: note = PA13R3P3-D10 Light Chain Variable region
DIVLSQSPAIMSASPGERVTMTCTASSSVSSSYLHWYQQKPGSSPKLWIYSTSNLASGVPARFSGSGSGTSYSLTISSMEAEDAATYYCHQYHRSPPTFGGGTKLELK

SEQ ID NO: 199 - DNA, 324 bp, synthetic construct; identical to SEQ ID NO: 7
  misc_feature 1..324: note = PA13R3P3-D10 Light Chain Constant region
  source 1..324: mol_type = other DNA

SEQ ID NO: 200 - protein, 107 aa, synthetic construct; identical to SEQ ID NO: 8
  REGION 1..107: note = PA13R3P3-D10 Light Chain Constant region

SEQ ID NO: 201 - DNA, 351 bp, synthetic construct
  misc_feature 1..351: note = PA13R3P3-D11 Heavy Chain Variable region
  source 1..351: mol_type = other DNA
gaggttcagctgcagcagtctggacctgagctgatgaagcctggggcttcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaactaccttccttcctggggccaagggactctggtcactgtctctgca

SEQ ID NO: 202 - protein, 117 aa, synthetic construct
  REGION 1..117: note = PA13R3P3-D11 Heavy Chain Variable region
EVQLQQSGPELMKPGASVKISCKASGYEFSYYWMNWVKQRPGQGLEWIGQIYPGDGETKYNGKFKGKATLTADKSSSTAYMQLSSLTSEDSAVYFCARSHGNYLPSWGQGTLVTVSA

SEQ ID NO: 203 - DNA, 993 bp, synthetic construct; identical to SEQ ID NO: 3
  misc_feature 1..993: note = PA13R3P3-D11 Heavy Chain Constant region
  source 1..993: mol_type = other DNA

SEQ ID NO: 204 - protein, 204 aa, synthetic construct; identical to SEQ ID NO: 68
  REGION 1..204: note = PA13R3P3-D11 Heavy Chain Constant region

SEQ ID NO: 205 - DNA, 324 bp, synthetic construct
  misc_feature 1..324: note = PA13R3P3-D11 Light Chain Variable region
  source 1..324: mol_type = other DNA
gacattgttctctcccagtctccagcaatcatgtccgcatctctaggggaacgggtcaccatgacctgcactgccagctcaagtgtaagttccagttacttacactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggaggggggaccaagctggagctgaaa

SEQ ID NO: 206 - protein, 108 aa, synthetic construct
  REGION 1..108: note = PA13R3P3-D11 Light Chain Variable region
DIVLSQSPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLELK

SEQ ID NO: 207 - DNA, 324 bp, synthetic construct; identical to SEQ ID NO: 7
  misc_feature 1..324: note = PA13R3P3-D11 Light Chain Constant region
  source 1..324: mol_type = other DNA

SEQ ID NO: 208 - protein, 107 aa, synthetic construct; identical to SEQ ID NO: 8
  REGION 1..107: note = PA13R3P3-D11 Light Chain Constant region

SEQ ID NO: 209 - DNA, 351 bp, synthetic construct
  misc_feature 1..351: note = PA13R3P3-F6 Heavy Chain Variable region
  source 1..351: mol_type = other DNA
gaggtccagctgcagcagtctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaactacattccttactggggccaagggactctggtcactgtctctgca

SEQ ID NO: 210 - protein, 117 aa, synthetic construct
  REGION 1..117: note = PA13R3P3-F6 Heavy Chain Variable region
EVQLQQSGAELLRPGSSVKISCKASGYEFSYYWMNWVKQRPGQGLEWIGQIYPGDGETKYNGKFKGKATLTADKSSSTAYMQLSSLTSEDSAVYFCARSHGNYIPYWGQGTLVTVSA

SEQ ID NO: 211 - DNA, 993 bp, synthetic construct; identical to SEQ ID NO: 3
  misc_feature 1..993: note = PA13R3P3-F6 Heavy Chain Constant region
  source 1..993: mol_type = other DNA

SEQ ID NO: 212 - protein, 204 aa, synthetic construct; identical to SEQ ID NO: 68
  REGION 1..204: note = PA13R3P3-F6 Heavy Chain Constant region

SEQ ID NO: 213 - DNA, 324 bp, synthetic construct
  misc_feature 1..324: note = PA13R3P3-F6 Light Chain Variable region
  source 1..324: mol_type = other DNA
gatattgttctctcccagtctccagcaatcatgtctgcatctctaggggagcgtgtcaccatgacctgcactgccagctcaagtgtaagttccagttacttacactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggaggggggaccaagctggagctgaaa

SEQ ID NO: 214 - protein, 108 aa, synthetic construct; identical to SEQ ID NO: 206
  REGION 1..108: note = PA13R3P3-F6 Light Chain Variable region

SEQ ID NO: 215 - DNA, 324 bp, synthetic construct; identical to SEQ ID NO: 7
  misc_feature 1..324: note = PA13R3P3-F6 Light Chain Constant region
  source 1..324: mol_type = other DNA

SEQ ID NO: 216 - protein, 107 aa, synthetic construct; identical to SEQ ID NO: 8
  REGION 1..107: note = PA13R3P3-F6 Light Chain Constant region

SEQ ID NO: 217 - DNA, 351 bp, synthetic construct
  misc_feature 1..351: note = PA13R3P4-C4 Heavy Chain Variable region
  source 1..351: mol_type = other DNA
gaggttcaactgcagcagtctggggctgagctgctgaggcctgggtcctcagtgaagatttcctgcaaggcttctggctatgaattcaattactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaactaccttccttactggggccaagggactctggtcactgtctctgca

SEQ ID NO: 218 - protein, 117 aa, synthetic construct
  REGION 1..117: note = PA13R3P4-C4 Heavy Chain Variable region
EVQLQQSGAELLRPGSSVKISCKASGYEFNYYWMNWVKQRPGQGLEWIGQIYPGDGETKYNGKFKGKATLTADKSSSTAYMQLSSLTSEDSAVYFCARSHGNYLPYWGQGTLVTVSA

SEQ ID NO: 219 - DNA, 993 bp, synthetic construct; identical to SEQ ID NO: 3
  misc_feature 1..993: note = PA13R3P4-C4 Heavy Chain Constant region
  source 1..993: mol_type = other DNA

SEQ ID NO: 220 - protein, 204 aa, synthetic construct; identical to SEQ ID NO: 68
  REGION 1..204: note = PA13R3P4-C4 Heavy Chain Constant region

SEQ ID NO: 221 - DNA, 333 bp, synthetic construct
  misc_feature 1..333: note = PA13R3P4-C4 Light Chain Variable region
  source 1..333: mol_type = other DNA
gacattgagctcacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcagcaaaataatgaggatcctccgacgttcggtggagggaccaagctggaaataaaa

SEQ ID NO: 222 - protein, 111 aa, synthetic construct
  REGION 1..111: note = PA13R3P4-C4 Light Chain Variable region
DIELTQSPASLAVSLGQRATISCRASESVDSYGNSFMHWYQQKPGQPPKLLIYLASNLESGVPARFSGSGSRTDFTLTIDPVEADDAATYYCQQNNEDPPTFGGGTKLEIK

SEQ ID NO: 223 - DNA, 324 bp, synthetic construct; identical to SEQ ID NO: 7
  misc_feature 1..324: note = PA13R3P4-C4 Light Chain Constant region
  source 1..324: mol_type = other DNA

SEQ ID NO: 224 - protein, 107 aa, synthetic construct; identical to SEQ ID NO: 8
  REGION 1..107: note = PA13R3P4-C4 Light Chain Constant region

SEQ ID NO: 225 - DNA, 351 bp, synthetic construct
  misc_feature 1..351: note = PA13R3P4-F8 Heavy Chain Variable region
  source 1..351: mol_type = other DNA
gaggtccagcttcagcagtctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaactaccttccttactggggccaagggactctggtcactgtctctgca

SEQ ID NO: 226 - protein, 117 aa, synthetic construct; identical to SEQ ID NO: 178
  REGION 1..117: note = PA13R3P4-F8 Heavy Chain Variable region

SEQ ID NO: 227 - DNA, 993 bp, synthetic construct; identical to SEQ ID NO: 3
  misc_feature 1..993: note = PA13R3P4-F8 Heavy Chain Constant region
  source 1..993: mol_type = other DNA

SEQ ID NO: 228 - protein, 204 aa, synthetic construct; identical to SEQ ID NO: 68
  REGION 1..204: note = PA13R3P4-F8 Heavy Chain Constant region

SEQ ID NO: 229 - DNA, 333 bp, synthetic construct
  misc_feature 1..333: note = PA13R3P4-F8 Light Chain Variable region
  source 1..333: mol_type = other DNA
gacattgtgctcacccaatctccagcttctttggctgtgtctctagggcagagggccaccatatcctgcagagccagtgaaagtgttgatagttatggcaatagttttatgcactggtaccagcagaaaccaggacagccacccaaactcctcatctatcttgcatccaacctagaacctggggtccctgccaggttcagtggcagtgggtctaggacagacttcaccctcaccattgatcctgtggaggctgatgatgctgcaacctattactgtcagcaaaataatgaggatcctccgacgttcggtggagggaccaagctggagctgaaa

SEQ ID NO: 230 - protein, 111 aa, synthetic construct
  REGION 1..111: note = PA13R3P4-F8 Light Chain Variable region
DIVLTQSPASLAVSLGQRATISCRASESVDSYGNSFMHWYQQKPGQPPKLLIYLASNLEPGVPARFSGSGSRTDFTLTIDPVEADDAATYYCQQNNEDPPTFGGGTKLELK

SEQ ID NO: 231 - DNA, 324 bp, synthetic construct; identical to SEQ ID NO: 7
  misc_feature 1..324: note = PA13R3P4-F8 Light Chain Constant region
  source 1..324: mol_type = other DNA

SEQ ID NO: 232 - protein, 107 aa, synthetic construct; identical to SEQ ID NO: 8
  REGION 1..107: note = PA13R3P4-F8 Light Chain Constant region

SEQ ID NO: 233 - DNA, 351 bp, synthetic construct
  misc_feature 1..351: note = PA13R3P4-G11 Heavy Chain Variable region
  source 1..351: mol_type = other DNA
gaggtgcagctccagcagtctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaactaccttccttactggggccaagggactctggtcactgtctctgca

SEQ ID NO: 234 - protein, 117 aa, synthetic construct; identical to SEQ ID NO: 178
  REGION 1..117: note = PA13R3P4-G11 Heavy Chain Variable region

SEQ ID NO: 235 - DNA, 993 bp, synthetic construct; identical to SEQ ID NO: 3
  misc_feature 1..993: note = PA13R3P4-G11 Heavy Chain Constant region
  source 1..993: mol_type = other DNA

SEQ ID NO: 236 - protein, 204 aa, synthetic construct; identical to SEQ ID NO: 68
  REGION 1..204: note = PA13R3P4-G11 Heavy Chain Constant region

SEQ ID NO: 237 - DNA, 330 bp, synthetic construct
  misc_feature 1..330: note = PA13R3P4-G11 Light Chain Variable region
  source 1..330: mol_type = other DNA
gatattgtgttaacacagtctcctgcttccttagctgtgtctctggggcagagggccaccatctcatgcagggccagccaaagtgtcagtacatctcgctatagttatatgcactggtaccaacagaaaccaggacagccacccaaactcctcatcaagtatgcatccaacctagaatctggggtccctgccaggttcagtggcagtgggtctgggacagacttcaccctcaacatccaccctgtggaggaggaggatattgcaacatattactgtcagcacagttgggagattcccacgttcggctcggggaccaagctggagctgaaa

SEQ ID NO: 238 - protein, 110 aa, synthetic construct
  REGION 1..110: note = PA13R3P4-G11 Light Chain Variable region
DIVLTQSPASLAVSLGQRATISCRASQSVSTSRYSYMHWYQQKPGQPPKLLIKYASNLESGVPARFSGSGSGTDFTLNIHPVEEEDIATYYCQHSWEIPTFGSGTKLELK

SEQ ID NO: 239 - DNA, 324 bp, synthetic construct; identical to SEQ ID NO: 7
  misc_feature 1..324: note = PA13R3P4-G11 Light Chain Constant region
  source 1..324: mol_type = other DNA

SEQ ID NO: 240 - protein, 107 aa, synthetic construct; identical to SEQ ID NO: 8
  REGION 1..107: note = PA13R3P4-G11 Light Chain Constant region

SEQ ID NO: 241 - DNA, 351 bp, synthetic construct
  misc_feature 1..351: note = PA13R3P4-H10 Heavy Chain Variable region
  source 1..351: mol_type = other DNA
gaggttcaactgcagcagtctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgatactaacttcaatggaaagttcaagggtaaagccacactgactgcagacaaatcctccagcacagcctacatgcagctcagcagcctaacatctgaggactctgcggtctatttctgtgcaagatcccatggtaaccacctagcttactggggccaagggactctggtcactgtctctgca

SEQ ID NO: 242 - protein, 117 aa, synthetic construct
  REGION 1..117: note = PA13R3P4-H10 Heavy Chain Variable region
EVQLQQSGAELLRPGSSVKISCKASGYEFSYYWMNWVKQRPGQGLEWIGQIYPGDGDTNFNGKFKGKATLTADKSSSTAYMQLSSLTSEDSAVYFCARSHGNHLAYWGQGTLVTVSA

SEQ ID NO: 243 - DNA, 993 bp, synthetic construct; identical to SEQ ID NO: 3
  misc_feature 1..993: note = PA13R3P4-H10 Heavy Chain Constant region
  source 1..993: mol_type = other DNA

SEQ ID NO: 244 - protein, 204 aa, synthetic construct; identical to SEQ ID NO: 68
  REGION 1..204: note = PA13R3P4-H10 Heavy Chain Constant region

SEQ ID NO: 245 - DNA, 324 bp, synthetic construct
  misc_feature 1..324: note = PA13R3P4-H10 Light Chain Variable region
  source 1..324: mol_type = other DNA
gatattgttctcaaccagtctccagcaatcatgtctgcatctctaggggaacgggtcaccatgacctgcactgccagctcaagtgtaagttccagttacttacactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggaggggggaccaagctggagctgaaa

SEQ ID NO: 246 - protein, 108 aa, synthetic construct
  REGION 1..108: note = PA13R3P4-H10 Light Chain Variable region
DIVLNQSPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLELK

SEQ ID NO: 247 - DNA, 324 bp, synthetic construct
  misc_feature 1..324: note = PA13R3P4-H10 Light Chain Constant region
  source 1..324: mol_type = other DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgtgaagtcacccatcagggcctgagcttgcccgtcacaaagagcttcaacaggggagagtgttaa

SEQ ID NO: 248 - protein, 107 aa, synthetic construct; identical to SEQ ID NO: 8
  REGION 1..107: note = PA13R3P4-H10 Light Chain Constant region

SEQ ID NO: 249 - DNA, 351 bp, synthetic construct
  misc_feature 1..351: note = PA15R3P3-A6 Heavy Chain Variable region
  source 1..351: mol_type = other DNA
gaggttcaactgcagcagtctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaactaccttgcttactggggccaagggactctggtcactgtctctgca

SEQ ID NO: 250 - protein, 117 aa, synthetic construct
  REGION 1..117: note = PA15R3P3-A6 Heavy Chain Variable region
EVQLQQSGAELLRPGSSVKISCKASGYEFSYYWMNWVKQRPGQGLEWIGQIYPGDGETKYNGKFKGKATLTADKSSSTAYMQLSSLTSEDSAVYFCARSHGNYLAYWGQGTLVTVSA

SEQ ID NO: 251 - DNA, 993 bp, synthetic construct; identical to SEQ ID NO: 3
  misc_feature 1..993: note = PA15R3P3-A6 Heavy Chain Constant region
  source 1..993: mol_type = other DNA

SEQ ID NO: 252 - protein, 204 aa, synthetic construct; identical to SEQ ID NO: 68
  REGION 1..204: note = PA15R3P3-A6 Heavy Chain Constant region

SEQ ID NO: 253 - DNA, 324 bp, synthetic construct
  misc_feature 1..324: note = PA15R3P3-A6 Light Chain Variable region
  source 1..324: mol_type = other DNA
gatattgtaatgacccagtctccagcaatcatgtctgcatctctaggggaacgggtcaccatgacctgcactgccagctcaagtgtaagttccagttacttacactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttccccacccacgttcggaggggggaccaagctggagctgaaa

SEQ ID NO: 254 - protein, 108 aa, synthetic construct
  REGION 1..108: note = PA15R3P3-A6 Light Chain Variable region
DIVMTQSPAIMSASLGERVTMTCTASSSVSSSYLHWYQQKPGSSPKLWIYSTSNLASGVPARFSGSGSGTSYSLTISSMEAEDAATYYCHQYHRSPPTFGGGTKLELK

SEQ ID NO: 255 - DNA, 324 bp, synthetic construct; identical to SEQ ID NO: 7
  misc_feature 1..324: note = PA15R3P3-A6 Light Chain Constant region
  source 1..324: mol_type = other DNA

SEQ ID NO: 256 - protein, 107 aa, synthetic construct; identical to SEQ ID NO: 8
  REGION 1..107: note = PA15R3P3-A6 Light Chain Constant region

SEQ ID NO: 257 - DNA, 351 bp, synthetic construct
  misc_feature 1..351: note = PA15R3P3-A7 Heavy Chain Variable region
  source 1..351: mol_type = other DNA
gaggttcagctgcagcagtctggacctgagctggtgaagcctgggtcttcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccagcacagcctacatgcagctcagcagcctaacatctgaggactctgcggtctatttctgtgcaagatcccatggtaaccaccttccttactggggccaagggactctggtcactgtctctgca

SEQ ID NO: 258 - protein, 117 aa, synthetic construct
  REGION 1..117: note = PA15R3P3-A7 Heavy Chain Variable region
EVQLQQSGPELVKPGSSVKISCKASGYEFSYYWMNWVKQRPGQGLEWIGQIYPGDGETKYNGKFKGKATLTADKSSSTAYMQLSSLTSEDSAVYFCARSHGNHLPYWGQGTLVTVSA

SEQ ID NO: 259 - DNA, 993 bp, synthetic construct; identical to SEQ ID NO: 3
  misc_feature 1..993: note = PA15R3P3-A7 Heavy Chain Constant region
  source 1..993: mol_type = other DNA

SEQ ID NO: 260 - protein, 204 aa, synthetic construct; identical to SEQ ID NO: 68
  REGION 1..204: note = PA15R3P3-A7 Heavy Chain Constant region

SEQ ID NO: 261 - DNA, 324 bp, synthetic construct
  misc_feature 1..324: note = PA15R3P3-A7 Light Chain Variable region
  source 1..324: mol_type = other DNA
gacattgttctcacccagtctccagcaatcatgtctgcatctctaggggaacgggtcaccctgacctgcactgccagctcaagtgtaagttccagttacttacactggtaccagcagaagccaggatcctcccccaaactctggatttatagcacttccaacctggcttctggagtcccagctcgcttcagtggcagtgggtctgggacctcttactctctcacaatcagcagcatggaggctgaagatgctgccacttattactgccaccagtatcatcgttccccacccacgttcggaggggggaccaagttggaaataaaa

SEQ ID NO: 262 - protein, 108 aa, synthetic construct
  REGION 1..108: note = PA15R3P3-A7 Light Chain Variable region
DIVLTQSPAIMSASLGERVTLTCTASSSVSSSYLHWYQQKPGSSPKLWIYSTSNLASGVPARFSGSGSGTSYSLTISSMEAEDAATYYCHQYHRSPPTFGGGTKLEIK

SEQ ID NO: 263 - DNA, 324 bp, synthetic construct; identical to SEQ ID NO: 7
  misc_feature 1..324: note = PA15R3P3-A7 Light Chain Constant region
  source 1..324: mol_type = other DNA

SEQ ID NO: 264 - protein, 107 aa, synthetic construct; identical to SEQ ID NO: 8
  REGION 1..107: note = PA15R3P3-A7 Light Chain Constant region

SEQ ID NO: 265 - DNA, 351 bp, synthetic construct
  misc_feature 1..351: note = PA15R3P3-C9 Heavy Chain Variable region
  source 1..351: mol_type = other DNA
gaggtccagctgcagcaatctggggctgagctgctgaggcctgggtcctcagtgaagatttcctgcaaggcttctggctatgaattcagttactactggatgaactgggtgaagcagaggcctggacagggtcttgagtggattggacagatttatcctggagatggtgaaactaagtacaatggaaagttcaagggtaaagccacactgactgcagacaaatcctccaacacagcctatatgcagctcagcagcctaacatctgaggactctgcggtctatttctgtgcaagatcccatggtaactaccttccttactggggccaagggactctggtcactgtctctgca

SEQ ID NO: 266 - protein, 117 aa, synthetic construct
  REGION 1..117: note = PA15R3P3-C9 Heavy Chain Variable region
EVQLQQSGAELLRPGSSVKISCKASGYEFSYYWMNWVKQRPGQGLEWIGQIYPGDGETKYNGKFKGKATLTADKSSNTAYMQLSSLTSEDSAVYFCARSHGNYLPYWGQGTLVTVSA

SEQ ID NO: 267 - DNA, 993 bp, synthetic construct; identical to SEQ ID NO: 3
  misc_feature 1..993: note = PA15R3P3-C9 Heavy Chain Constant region
  source 1..993: mol_type = other DNA

SEQ ID NO: 268 - protein, 204 aa, synthetic construct; identical to SEQ ID NO: 68
  REGION 1..204: note = PA15R3P3-C9 Heavy Chain Constant region

SEQ ID NO: 269 - DNA, 324 bp, synthetic construct
  misc_feature 1..324: note = PA15R3P3-C9 Light Chain Variable region
  source 1..324: mol_type = other DNA
ggcattctgatgacccagtctccagcaatcatgtctgcatctctaggggaacgggtcaccatgacctgcactgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaatcagcagcatggaggctgaagatgctgccacttattactgccaccagtttcatcgttccccacccacgttcggaggggggaccaagctggagctgaaa

SEQ ID NO: 270 - protein, 108 aa, synthetic construct
  REGION 1..108: note = PA15R3P3-C9 Light Chain Variable region
GILMTQSPAIMSASLGERVTMTCTASSSISSNYLHWYQQKPGFSPKLLIYRTSNLASGVPARFSGSGSGTSYSLTISSMEAEDAATYYCHQFHRSPPTFGGGTKLELK

SEQ ID NO: 271 - DNA, 324 bp, synthetic construct; identical to SEQ ID NO: 7
  misc_feature 1..324: note = PA15R3P3-C9 Light Chain Constant region
  source 1..324: mol_type = other DNA

SEQ ID NO: 272 - protein, 107 aa, synthetic construct; identical to SEQ ID NO: 8
  REGION 1..107: note = PA15R3P3-C9 Light Chain Constant region

SEQ ID NO: 273 - DNA, 351 bp, synthetic construct; identical to SEQ ID NO: 25
  misc_feature 1..351: note = PA16R3-P2G6 Heavy Chain Variable region
  source 1..351: mol_type = other DNA

SEQ ID NO: 274 - protein, 117 aa, synthetic construct; identical to SEQ ID NO: 26
  REGION 1..117: note = PA16R3-P2G6 Heavy Chain Variable region

SEQ ID NO: 275 - DNA, 993 bp, synthetic construct; identical to SEQ ID NO: 3
  misc_feature 1..993: note = PA16R3-P2G6 Heavy Chain Constant region
  source 1..993: mol_type = other DNA

SEQ ID NO: 276 - protein, 204 aa, synthetic construct; identical to SEQ ID NO: 68
  REGION 1..204: note = PA16R3-P2G6 Heavy Chain Constant region

SEQ ID NO: 277 - DNA, 324 bp, synthetic construct
  misc_feature 1..324: note = PA16R3-P2G6 Light Chain Variable region
  source 1..324: mol_type = other DNA
gatattgagctgacccaatctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttatttctgccagcagtggagtagtttcccgctcacgttcggtggagggaccaagctggagctgaaa

SEQ ID NO: 278 - protein, 108 aa, synthetic construct
  REGION 1..108: note = PA16R3-P2G6 Light Chain Variable region
DIELTQSPAIMSASLGERVTMTCTASSSVSSSYLHWYQQKPGSSPKLWIYSTSNLASGVPARFSGSGSGTSYSLTISSMEAEDAATYFCQQWSSFPLTFGGGTKLELK

SEQ ID NO: 279 - DNA, 324 bp, synthetic construct; identical to SEQ ID NO: 7
  misc_feature 1..324: note = PA16R3-P2G6 Light Chain Constant region
  source 1..324: mol_type = other DNA

SEQ ID NO: 280 - protein, 107 aa, synthetic construct; identical to SEQ ID NO: 8
  REGION 1..107: note = PA16R3-P2G6 Light Chain Constant region

SEQ ID NO: 281 - DNA, 351 bp, synthetic construct; identical to SEQ ID NO: 25
  misc_feature 1..351: note = PA16R3-P1A6 Heavy Chain Variable region
  source 1..351: mol_type = other DNA

SEQ ID NO: 282 - protein, 117 aa, synthetic construct; identical to SEQ ID NO: 26
  REGION 1..117: note = PA16R3-P1A6 Heavy Chain Variable region

SEQ ID NO: 283 - DNA, 993 bp, synthetic construct; identical to SEQ ID NO: 3
  misc_feature 1..993: note = PA16R3-P1A6 Heavy Chain Constant region
  source 1..993: mol_type = other DNA

SEQ ID NO: 284 - protein, 204 aa, synthetic construct; identical to SEQ ID NO: 68
  REGION 1..204: note = PA16R3-P1A6 Heavy Chain Constant region

SEQ ID NO: 285 - DNA, 324 bp, synthetic construct
  misc_feature 1..324: note = PA16R3-P1A6 Light Chain Variable region
  source 1..324: mol_type = other DNA
gatattgttctcaaccagtctccagcaatcatgtctgcatctctaggggaacgggtcaccatgacctgcactgccagctcaagtgtaagttccagttacttacactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagcatcatcgttccccacccacgttcggaggggggacaaagttggaaataaaa

SEQ ID NO: 286 - protein, 108 aa, synthetic construct
  REGION 1..108: note = PA16R3-P1A6 Light Chain Variable region
DIVLNQSPAIMSASLGERVTMTCTASSSVSSSYLHWYQQKPGSSPKLWIYSTSNLASGVPARFSGSGSGTSYSLTISSMEAEDAATYYCHQHHRSPPTFGGGTKLEIK

SEQ ID NO: 287 - DNA, 324 bp, synthetic construct; identical to SEQ ID NO: 7
  misc_feature 1..324: note = PA16R3-P1A6 Light Chain Constant region
  source 1..324: mol_type = other DNA

SEQ ID NO: 288 - protein, 107 aa, synthetic construct; identical to SEQ ID NO: 8
  REGION 1..107: note = PA16R3-P1A6 Light Chain Constant region

SEQ ID NO: 289 - DNA, 351 bp, synthetic construct; identical to SEQ ID NO: 25
  misc_feature 1..351: note = PA16R3-P1B5 Heavy Chain Variable region
  source 1..351: mol_type = other DNA

SEQ ID NO: 290 - protein, 117 aa, synthetic construct; identical to SEQ ID NO: 26
  REGION 1..117: note = PA16R3-P1B5 Heavy Chain Variable region

SEQ ID NO: 291 - DNA, 993 bp, synthetic construct; identical to SEQ ID NO: 3
  misc_feature 1..993: note = PA16R3-P1B5 Heavy Chain Constant region
  source 1..993: mol_type = other DNA

SEQ ID NO: 292 - protein, 204 aa, synthetic construct; identical to SEQ ID NO: 68
  REGION 1..204: note = PA16R3-P1B5 Heavy Chain Constant region

SEQ ID NO: 293 - DNA, 334 bp, synthetic construct
  misc_feature 1..334: note = PA16R3-P1B5 Light Chain Variable region
  source 1..334: mol_type = other DNA
gatattgtgctaacacagtctcctgcttccttagctgtatctctggggcagagggccaccatctcatgcagggccagcaaaagtgtcagtacatctggctatagttatatgcactggttccagcagaaaccaggacagccacccaaactcctcatcaagtatgcatccaacctagaatctggggtccctgccaggttcagtggcagtgggtctgggacagacttcaccctcaacatccatcctgtggaggaggaggatgctgcaacctattactgtcagcacggttgggagcttccattcacgttcggctcggggaccaagctggagctgaaac

SEQ ID NO: 294 - protein, 111 aa, synthetic construct
  REGION 1..111: note = PA16R3-P1B5 Light Chain Variable region
DIVLTQSPASLAVSLGQRATISCRASKSVSTSGYSYMHWFQQKPGQPPKLLIKYASNLESGVPARFSGSGSGTDFTLNIHPVEEEDAATYYCQHGWELPFTFGSGTKLELK

SEQ ID NO: 295 - DNA, 324 bp, synthetic construct; identical to SEQ ID NO: 7
  misc_feature 1..324: note = PA16R3-P1B5 Light Chain Constant region
  source 1..324: mol_type = other DNA

SEQ ID NO: 296 - protein, 107 aa, synthetic construct; identical to SEQ ID NO: 8
  REGION 1..107: note = PA16R3-P1B5 Light Chain Constant region

SEQ ID NO: 297 - DNA, 351 bp, synthetic construct; identical to SEQ ID NO: 25
  misc_feature 1..351: note = PA16R3-P1E5 Heavy Chain Variable region
  source 1..351: mol_type = other DNA

SEQ ID NO: 298 - protein, 117 aa, synthetic construct; identical to SEQ ID NO: 26
  REGION 1..117: note = PA16R3-P1E5 Heavy Chain Variable region

SEQ ID NO: 299 - DNA, 993 bp, synthetic construct; identical to SEQ ID NO: 3
  misc_feature 1..993: note = PA16R3-P1E5 Heavy Chain Constant region
  source 1..993: mol_type = other DNA

SEQ ID NO: 300 - protein, 204 aa, synthetic construct; identical to SEQ ID NO: 68
  REGION 1..204: note = PA16R3-P1E5 Heavy Chain Constant region

SEQ ID NO: 301 - DNA, 324 bp, synthetic construct
  misc_feature 1..324: note = PA16R3-P1E5 Light Chain Variable region
  source 1..324: mol_type = other DNA
gacattgtgatgacccagtctccagcaatcatgtctgcatctctaggggaacgggtcaccctgacctgcactgccagttcaagtgttagttccagttacttacactggtaccagcagaagccaggatcctcccccaaactctggatttatagcacttccaacctggcttctggagtcccagctcgcttcagtggcagtgggtctgggacctcttactctctcacaatcagcagcatggaggctgaagatgctgccacttattactgccaccagtatcatcgttccccacccacgttcggaggggggaccaagctggagctgaaa

SEQ ID NO: 302 - protein, 108 aa, synthetic construct
  REGION 1..108: note = PA16R3-P1E5 Light Chain Variable region
DIVMTQSPAIMSASLGERVTLTCTASSSVSSSYLHWYQQKPGSSPKLWIYSTSNLASGVPARFSGSGSGTSYSLTISSMEAEDAATYYCHQYHRSPPTFGGGTKLELK

SEQ ID NO: 303 - DNA, 324 bp, synthetic construct; identical to SEQ ID NO: 7
  misc_feature 1..324: note = PA16R3-P1E5 Light Chain Constant region
  source 1..324: mol_type = other DNA

SEQ ID NO: 304 - protein, 107 aa, synthetic construct; identical to SEQ ID NO: 8
  REGION 1..107: note = PA16R3-P1E5 Light Chain Constant region

SEQ ID NO: 305 - DNA, 351 bp, synthetic construct; identical to SEQ ID NO: 25
  misc_feature 1..351: note = PA16R3-P1H5 Heavy Chain Variable region
  source 1..351: mol_type = other DNA

SEQ ID NO: 306 - protein, 117 aa, synthetic construct; identical to SEQ ID NO: 26
  REGION 1..117: note = PA16R3-P1H5 Heavy Chain Variable region

SEQ ID NO: 307 - DNA, 993 bp, synthetic construct; identical to SEQ ID NO: 3
  misc_feature 1..993: note = PA16R3-P1H5 Heavy Chain Constant region
  source 1..993: mol_type = other DNA

SEQ ID NO: 308 - protein, 204 aa, synthetic construct; identical to SEQ ID NO: 68
  REGION 1..204: note = PA16R3-P1H5 Heavy Chain Constant region

SEQ ID NO: 309 - DNA, 333 bp, synthetic construct
  misc_feature 1..333: note = PA16R3-P1H5 Light Chain Variable region
  source 1..333: mol_type = other DNA
gatattgtgatgacccagtctccagcttctttggctgtgtctctagggcagagggccaccatatcctgcagagccagtgaaagtgttgatagttatggcaatagttttatgcactggtaccagcagaaagcaggacagccacccaaactcctcatctatcttgcatccaacctagaatctggggtccctgccaggttcagtggcagtgggtctaggacagacttcaccctcaccattgatcctgtggaggctgatgatgctgcaacctattactgtcaacaacattatagtactcctccgacgttcggtggagggacaaagttggaaataaaa

SEQ ID NO: 310 - protein, 111 aa, synthetic construct
  REGION 1..111: note = PA16R3-P1H5 Light Chain Variable region
DIVMTQSPASLAVSLGQRATISCRASESVDSYGNSFMHWYQQKAGQPPKLLIYLASNLESGVPARFSGSGSRTDFTLTIDPVEADDAATYYCQQHYSTPPTFGGGTKLEIK

SEQ ID NO: 311 - DNA, 324 bp, synthetic construct; identical to SEQ ID NO: 7
  misc_feature 1..324: note = PA16R3-P1H5 Light Chain Constant region
  source 1..324: mol_type = other DNA

SEQ ID NO: 312 - protein, 107 aa, synthetic construct; identical to SEQ ID NO: 8
  REGION 1..107: note = PA16R3-P1H5 Light Chain Constant region

SEQ ID NO: 313 - DNA, 351 bp, synthetic construct; identical to SEQ ID NO: 25
  misc_feature 1..351: note = PA18R3-P1D8 Heavy Chain Variable region
  source 1..351: mol_type = other DNA

SEQ ID NO: 314 - protein, 117 aa, synthetic construct; identical to SEQ ID NO: 26
  REGION 1..117: note = PA18R3-P1D8 Heavy Chain Variable region

SEQ ID NO: 315 - DNA, 993 bp, synthetic construct; identical to SEQ ID NO: 3
  misc_feature 1..993: note = PA18R3-P1D8 Heavy Chain Constant region
  source 1..993: mol_type = other DNA

SEQ ID NO: 316 - protein, 204 aa, synthetic construct; identical to SEQ ID NO: 68
  REGION 1..204: note = PA18R3-P1D8 Heavy Chain Constant region

SEQ ID NO: 317 - DNA, 324 bp, synthetic construct
  misc_feature 1..324: note = PA18R3-P1D8 Light Chain Variable region
  source 1..324: mol_type = other DNA
gatattgtgataactcagtctccagcaatcatgtctgcatctctaggggaacgggtcaccatgacctgcactgccagctcaagtgtaagttccagttacttacactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccatttattactgccagcagtggaattatcctcctatcacgttcggctcggggaccaagctggaaatcaaa

SEQ ID NO: 318 - protein, 108 aa, synthetic construct
  REGION 1..108: note = PA18R3-P1D8 Light Chain Variable region
DIVITQSPAIMSASLGERVTMTCTASSSVSSSYLHWYQQKPGSSPKLWIYSTSNLASGVPARFSGSGSGTSYSLTISSMEAEDAAIYYCQQWNYPPITFGSGTKLEIK

SEQ ID NO: 319 - DNA, 324 bp, synthetic construct; identical to SEQ ID NO: 7
  misc_feature 1..324: note = PA18R3-P1D8 Light Chain Constant region
  source 1..324: mol_type = other DNA

SEQ ID NO: 320 - protein, 107 aa, synthetic construct; identical to SEQ ID NO: 8
  REGION 1..107: note = PA18R3-P1D8 Light Chain Constant region

SEQ ID NO: 321 - DNA, 351 bp, synthetic construct; identical to SEQ ID NO: 25
  misc_feature 1..351: note = PA18R3-P1E5 Heavy Chain Variable region
  source 1..351: mol_type = other DNA

SEQ ID NO: 322 - protein, 117 aa, synthetic construct; identical to SEQ ID NO: 26
  REGION 1..117: note = PA18R3-P1E5 Heavy Chain Variable region

SEQ ID NO: 323 - DNA, 993 bp, synthetic construct; identical to SEQ ID NO: 3
  misc_feature 1..993: note = PA18R3-P1E5 Heavy Chain Constant region
  source 1..993: mol_type = other DNA

SEQ ID NO: 324 - protein, 204 aa, synthetic construct; identical to SEQ ID NO: 68
  REGION 1..204: note = PA18R3-P1E5 Heavy Chain Constant region

SEQ ID NO: 325 - DNA, 324 bp, synthetic construct
  misc_feature 1..324: note = PA18R3-P1E5 Light Chain Variable region
  source 1..324: mol_type = other DNA
gacattgtaatgacccagtctccagcaatcatgtctgcatctccaggggagaaggtcaccataacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccattcacgttcggctcggggaccaagctggaaataaaa

SEQ ID NO: 326 - protein, 108 aa, synthetic construct
  REGION 1..108: note = PA18R3-P1E5 Light Chain Variable region
DIVMTQSPAIMSASPGEKVTITCSASSSISSNYLHWYQQKPGFSPKLLIYRTSNLASGVPARFSGSGSGTSYSLTIGTMEAEDVATYYCQQGSSIPFTFGSGTKLEIK

SEQ ID NO: 327 - DNA, 324 bp, synthetic construct; identical to SEQ ID NO: 7
  misc_feature 1..324: note = PA18R3-P1E5 Light Chain Constant region
  source 1..324: mol_type = other DNA

SEQ ID NO: 328 - protein, 107 aa, synthetic construct; identical to SEQ ID NO: 8
  REGION 1..107: note = PA18R3-P1E5 Light Chain Constant region

SEQ ID NO: 329 - DNA, 351 bp, synthetic construct; identical to SEQ ID NO: 25
  misc_feature 1..351: note = PA18R3-P1H5 Heavy Chain Variable region
  source 1..351: mol_type = other DNA

SEQ ID NO: 330 - protein, 117 aa, synthetic construct; identical to SEQ ID NO: 26
  REGION 1..117: note = PA18R3-P1H5 Heavy Chain Variable region

SEQ ID NO: 331 - DNA, 993 bp, synthetic construct; identical to SEQ ID NO: 3
  misc_feature 1..993: note = PA18R3-P1H5 Heavy Chain Constant region
  source 1..993: mol_type = other DNA

SEQ ID NO: 332 - protein, 204 aa, synthetic construct; identical to SEQ ID NO: 68
  REGION 1..204: note = PA18R3-P1H5 Heavy Chain Constant region

SEQ ID NO: 333 - DNA, 321 bp, synthetic construct
  misc_feature 1..321: note = PA18R3-P1H5 Light Chain Variable region
  source 1..321: mol_type = other DNA
gacattgttctcaaccagtctccagcaatcatgtctgcatctctaggggaacgggtcaccatgacctgcactgccagctcaagtgtaagttccagttacttgcactggtaccagcagaagccaggatcctcccccaaactctggatttatagcacatccaacctggcttctggagtcccagctcgcttcagtggcagtgggtctgggacctcttactctctcacaatcagcagcatggaggctgaagatgctgccacttattactgccaccagtatcatcgttcccacacgttcggaggggggaccaaactggaaatcaaa

SEQ ID NO: 334 - protein, 107 aa, synthetic construct
  REGION 1..107: note = PA18R3-P1H5 Light Chain Variable region
DIVLNQSPAIMSASLGERVTMTCTASSSVSSSYLHWYQQKPGSSPKLWIYSTSNLASGVPARFSGSGSGTSYSLTISSMEAEDAATYYCHQYHRSHTFGGGTKLEIK

SEQ ID NO: 335 - DNA, 324 bp, synthetic construct; identical to SEQ ID NO: 7
  misc_feature 1..324: note = PA18R3-P1H5 Light Chain Constant region
  source 1..324: mol_type = other DNA

SEQ ID NO: 336 - protein, 107 aa, synthetic construct; identical to SEQ ID NO: 8
  REGION 1..107: note = PA18R3-P1H5 Light Chain Constant region

SEQ ID NO: 337 - protein, 108 aa, synthetic construct
  REGION 1..108: note = Humanized P1C4 light chain variable region
EIVLTQSPATLSLSPGERATLSCRASQSVSSSYLHWYQQKPGQAPRLLIYSTSNLASGIPARFSGSGSGTDFTLTISRLEPEDFAVYYCHQYHRSPPTFGQGTKVEIK

SEQ ID NO: 338 - protein, 215 aa, synthetic construct
  REGION 1..215: note = Humanized P1C4 full light chain
EIVLTQSPATLSLSPGERATLSCRASQSVSSSYLHWYQQKPGQAPRLLIYSTSNLASGIPARFSGSGSGTDFTLTISRLEPEDFAVYYCHQYHRSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 339 - protein, 117 aa, synthetic construct
  REGION 1..117: note = Humanized P1C4-H0 heavy chain variable region
QVQLVQSGAEVKKPGSSVKVSCKASGGTFSYYWMNWVRQAPGQGLEWMGQIYPGDGETKYAQKFQGRVTITADKSTSTAYMELSSLRSEDTAVYYCARSHGNYLPYWGQGTLVTVSS

SEQ ID NO: 340 - protein, 117 aa, synthetic construct
  REGION 1..117: note = Humanized P1C4-H2 heavy chain variable region
QVQLVQSGAEVKKPGSSVKVSCKASGYEFSYYWMNWVRQAPGQGLEWMGQIYPGDGETKYAQKFQGRVTITADKSTSTAYMELSSLRSEDTAVYYCARSHGNYLPYWGQGTLVTVSS

SEQ ID NO: 341 - protein, 117 aa, synthetic construct
  REGION 1..117: note = Humanized P1C4-H4 heavy chain variable region
QVQLVQSGAEVKKPGSSVKVSCKASGYEFSYYWMNWVRQAPGQGLEWMGQIYPGDGETKYNGKFQGRVTITADKSTSTAYMELSSLRSEDTAVYYCARSHGNYLPYWGQGTLVTVSS

SEQ ID NO: 342 - protein, 330 aa, synthetic construct [sequence omitted]

SEQ ID NO: 343 - protein, 444 aa, synthetic construct
  REGION 1..444: note = Humanized P1C4 H0 IgG2-4 Hybrid
QVQLVQSGAEVKKPGSSVKVSCKASGGTFSYYWMNWVRQAPGQGLEWMGQIYPGDGETKYAQKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 344 - protein, 443 aa, synthetic construct
  REGION 1..443: note = Humanized P1C4 H0 IgG2A330S/P331S
QVQLVQSGAEVKKPGSSVKVSCKASGGTFSYYWMNWVRQAPGQGLEWMGQIYPGDGETKYAQKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 345 - protein, 444 aa, synthetic construct
  REGION 1..444: note = Humanized P1C4 H0 IgG4S228P
QVQLVQSGAEVKKPGSSVKVSCKASGGTFSYYWMNWVRQAPGQGLEWMGQIYPGDGETKYAQKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 346 - protein, 444 aa, synthetic construct
  REGION 1..444: note = Humanized P1C4 H2 IgG2-4 Hybrid
QVQLVQSGAEVKKPGSSVKVSCKASGYEFSYYWMNWVRQAPGQGLEWMGQIYPGDGETKYAQKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 347 - protein, 443 aa, synthetic construct
  REGION 1..443: note = Humanized P1C4 H2 IgG2A330S/P331S
QVQLVQSGAEVKKPGSSVKVSCKASGYEFSYYWMNWVRQAPGQGLEWMGQIYPGDGETKYAQKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 348 - protein, 444 aa, synthetic construct
  REGION 1..444: note = Humanized P1C4 H2 IgG4S228P
QVQLVQSGAEVKKPGSSVKVSCKASGYEFSYYWMNWVRQAPGQGLEWMGQIYPGDGETKYAQKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 349 - protein, 444 aa, synthetic construct
  REGION 1..444: note = Humanized P1C4 H4 IgG2-4 Hybrid
QVQLVQSGAEVKKPGSSVKVSCKASGYEFSYYWMNWVRQAPGQGLEWMGQIYPGDGETKYNGKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 350 - protein, 443 aa, synthetic construct
  REGION 1..443: note = Humanized P1C4 H4 IgG2A330S/P331S
QVQLVQSGAEVKKPGSSVKVSCKASGYEFSYYWMNWVRQAPGQGLEWMGQIYPGDGETKYNGKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 351 - protein, 444 aa, synthetic construct
  REGION 1..444: note = Humanized P1C4 H4 IgG4S228P
QVQLVQSGAEVKKPGSSVKVSCKASGYEFSYYWMNWVRQAPGQGLEWMGQIYPGDGETKYNGKFQGRVTITADKSTSTAYMELSSLRSEDTAVYYCARSHGNYLPYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 352 - protein, 5 aa, Mus sp.
YYWMN

SEQ ID NO: 353 - protein, 11 aa, Mus sp.
QIYPGDGETKY

SEQ ID NO: 354 - protein, 8 aa, Mus sp.
SHGNYLPY

SEQ ID NO: 355 - protein, 5 aa, Mus sp.
SSYLH

SEQ ID NO: 356 - protein, 7 aa, Mus sp.
STSNLAS

SEQ ID NO: 357 - protein, 10 aa, Mus sp.
HQYHRSPPTF

SEQ ID NO: 358-432 - skipped (75 entries)

SEQ ID NO: 433 - protein, 326 aa, synthetic construct
  REGION 1..326: note = IgG2 heavy constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 434 - protein, 327 aa, synthetic construct
  REGION 1..327: note = IgG4 heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 435 - protein, 327 aa, synthetic construct
  REGION 1..327: note = Hybrid IgG2/4 heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 436 - protein, 443 aa, synthetic construct; identical to SEQ ID NO: 347
  REGION 1..443: note = P1C4-H2-IgG2

SEQ ID NO: 437 - protein, 444 aa, synthetic construct; identical to SEQ ID NO: 348
  REGION 1..444: note = P1C4-H2-IgG4

SEQ ID NO: 438 - protein, 444 aa, synthetic construct; identical to SEQ ID NO: 346
  REGION 1..444: note = P1C4-H2-IgG2/4

SEQ ID NO: 439 - protein, 443 aa, synthetic construct; identical to SEQ ID NO: 350
  REGION 1..443: note = P1C4-H4-IgG2

SEQ ID NO: 440 - protein, 444 aa, synthetic construct; identical to SEQ ID NO: 351
  REGION 1..444: note = P1C4-H4-IgG4

SEQ ID NO: 441 - protein, 444 aa, synthetic construct; identical to SEQ ID NO: 349
  REGION 1..444: note = P1C4-H4-IgG2/4

SEQ ID NO: 442 - protein, 215 aa, synthetic construct; identical to SEQ ID NO: 338
  REGION 1..215: note = P1C4-Lc humanized

SEQ ID NO: 443 - protein, 5 aa, synthetic construct
  REGION 1..5: note = Heavy chain CDR1 sequence mut
HYWMN

SEQ ID NO: 444 - protein, 5 aa, synthetic construct
  REGION 1..5: note = Heavy chain CDR1 sequence mut
IYWMN

SEQ ID NO: 445 - protein, 5 aa, synthetic construct
  REGION 1..5: note = Heavy chain CDR1 sequence mut
WYWMN

SEQ ID NO: 446 - protein, 5 aa, synthetic construct
  REGION 1..5: note = Heavy chain CDR1 sequence mut
YFWMN

SEQ ID NO: 447 - protein, 5 aa, synthetic construct
  REGION 1..5: note = Heavy chain CDR1 sequence mut
YKWMN

SEQ ID NO: 448 - protein, 5 aa, synthetic construct
  REGION 1..5: note = Heavy chain CDR1 sequence mut
YNWMN

SEQ ID NO: 449 - protein, 5 aa, synthetic construct
  REGION 1..5: note = Heavy chain CDR1 sequence mut
YYAMN

SEQ ID NO: 450 - protein, 5 aa, synthetic construct
  REGION 1..5: note = Heavy chain CDR1 sequence mut
YYFMN

SEQ ID NO: 451 - protein, 5 aa, synthetic construct
  REGION 1..5: note = Heavy chain CDR1 sequence mut
YYWAN

SEQ ID NO: 452 - protein, 5 aa, synthetic construct
  REGION 1..5: note = Heavy chain CDR1 sequence mut
YYWEN

SEQ ID NO: 453 - protein, 5 aa, synthetic construct
  REGION 1..5: note = Heavy chain CDR1 sequence mut
YYWFN

SEQ ID NO: 454 - protein, 5 aa, synthetic construct
  REGION 1..5: note = Heavy chain CDR1 sequence mut
YYWLN

SEQ ID NO: 455 - protein, 5 aa, synthetic construct
  REGION 1..5: note = Heavy chain CDR1 sequence mut
YYWNN

SEQ ID NO: 456 - protein, 5 aa, synthetic construct
  REGION 1..5: note = Heavy chain CDR1 sequence mut
YYWQN

SEQ ID NO: 457 - protein, 5 aa, synthetic construct
  REGION 1..5: note = Heavy chain CDR1 sequence mut
YYWTN

SEQ ID NO: 458 - protein, 5 aa, synthetic construct
  REGION 1..5: note = Heavy chain CDR1 sequence mut
YYWVN

SEQ ID NO: 459 - protein, 5 aa, synthetic construct
  REGION 1..5: note = Heavy chain CDR1 sequence mut
YYWMI

SEQ ID NO: 460 - protein, 5 aa, synthetic construct
  REGION 1..5: note = Heavy chain CDR1 sequence mut
YYWMK

SEQ ID NO: 461 - protein, 5 aa, synthetic construct
  REGION 1..5: note = Heavy chain CDR1 sequence mut
YYWML

SEQ ID NO: 462 - protein, 5 aa, synthetic construct
  REGION 1..5: note = Heavy chain CDR1 sequence mut
YYWMS

SEQ ID NO: 463 - protein, 5 aa, synthetic construct
  REGION 1..5: note = Heavy chain CDR1 sequence mut
YYWMW

SEQ ID NO: 464 - protein, 11 aa, synthetic construct
  REGION 1..11: note = Heavy chain CDR2 sequence mut
DIYPGDGETKY

SEQ ID NO: 465 - protein, 11 aa, synthetic construct
  REGION 1..11: note = Heavy chain CDR2 sequence mut
EIYPGDGETKY

SEQ ID NO: 466 - protein, 11 aa, synthetic construct
  REGION 1..11: note = Heavy chain CDR2 sequence mut
QAYPGDGETKY

SEQ ID NO: 467 - protein, 11 aa, synthetic construct
  REGION 1..11: note = Heavy chain CDR2 sequence mut
QDYPGDGETKY

SEQ ID NO: 468 - protein, 11 aa, synthetic construct
  REGION 1..11: note = Heavy chain CDR2 sequence mut
QEYPGDGETKY

SEQ ID NO: 469 - protein, 11 aa, synthetic construct
  REGION 1..11: note = Heavy chain CDR2 sequence mut
QFYPGDGETKY

SEQ ID NO: 470 - protein, 11 aa, synthetic construct
  REGION 1..11: note = Heavy chain CDR2 sequence mut
QGYPGDGETKY

SEQ ID NO: 471 - protein, 11 aa, synthetic construct
  REGION 1..11: note = Heavy chain CDR2 sequence mut
QHYPGDGETKY

SEQ ID NO: 472 - protein, 11 aa, synthetic construct
  REGION 1..11: note = Heavy chain CDR2 sequence mut
QKYPGDGETKY

SEQ ID NO: 473 - protein, 11 aa, synthetic construct
  REGION 1..11: note = Heavy chain CDR2 sequence mut
QLYPGDGETKY

SEQ ID NO: 474 - protein, 11 aa, synthetic construct
  REGION 1..11: note = Heavy chain CDR2 sequence mut
QNYPGDGETKY

SEQ ID NO: 475 - protein, 11 aa, synthetic construct
  REGION 1..11: note = Heavy chain CDR2 sequence mut
QQYPGDGETKY

SEQ ID NO: 476 - protein, 11 aa, synthetic construct
  REGION 1..11: note = Heavy chain CDR2 sequence mut
QRYPGDGETKY

SEQ ID NO: 477 - protein, 11 aa, synthetic construct
  REGION 1..11: note = Heavy chain CDR2 sequence mut
QSYPGDGETKY

SEQ ID NO: 478 - protein, 11 aa, synthetic construct
  REGION 1..11: note = Heavy chain CDR2 sequence mut
QTYPGDGETKY

SEQ ID NO: 479 - protein, 11 aa, synthetic construct
  REGION 1..11: note = Heavy chain CDR2 sequence mut
QWYPGDGETKY

SEQ ID NO: 480 - protein, 11 aa, synthetic construct
  REGION 1..11: note = Heavy chain CDR2 sequence mut
QYYPGDGETKY

SEQ ID NO: 481 - protein, 11 aa, synthetic construct
  REGION 1..11: note = Heavy chain CDR2 sequence mut
QIFPGDGETKY

SEQ ID NO: 482 - protein, 11 aa, synthetic construct
  REGION 1..11: note = Heavy chain CDR2 sequence mut
QIYAGDGETKY

SEQ ID NO: 483 - protein, 11 aa, synthetic construct
  REGION 1..11: note = Heavy chain CDR2 sequence mut
QIYFGDGETKY

SEQ ID NO: 484 - protein, 11 aa, synthetic construct
  REGION 1..11: note = Heavy chain CDR2 sequence mut
QIYHGDGETKY

SEQ ID NO: 485 - protein, 11 aa, synthetic construct
  REGION 1..11: note = Heavy chain CDR2 sequence mut
QIYKGDGETKY

SEQ ID NO: 486 - protein, 11 aa, synthetic construct
  REGION 1..11: note = Heavy chain CDR2 sequence mut
QIYPADGETKY

SEQ ID NO: 487 - protein, 11 aa, synthetic construct
  REGION 1..11: note = Heavy chain CDR2 sequence mut
QIYPCDGETKY

SEQ ID NO: 488 - protein, 11 aa, synthetic construct
  REGION 1..11: note = Heavy chain CDR2 sequence mut
QIYPDDGETKY

SEQ ID NO: 489 - protein, 11 aa, synthetic construct
  REGION 1..11: note = Heavy chain CDR2 sequence mut
QIYPEDGETKY

SEQ ID NO: 490 - protein, 11 aa, synthetic construct
  REGION 1..11: note = Heavy chain CDR2 sequence mut
QIYPFDGETKY

SEQ ID NO: 491 - protein, 11 aa, synthetic construct
  REGION 1..11: note = Heavy chain CDR2 sequence mut
QIYPHDGETKY

SEQ ID NO: 492 - protein, 11 aa, synthetic construct
  REGION 1..11: note = Heavy chain CDR2 sequence mut
QIYPIDGETKY

SEQ ID NO: 493 - protein, 11 aa, synthetic construct
  REGION 1..11: note = Heavy chain CDR2 sequence mut
QIYPKDGETKY

SEQ ID NO: 494 - protein, 11 aa, synthetic construct
  REGION 1..11: note = Heavy chain CDR2 sequence mut
QIYPLDGETKY

SEQ ID NO: 495 - protein, 11 aa, synthetic construct
  REGION 1..11: note = Heavy chain CDR2 sequence mut
QIYPMDGETKY

SEQ ID NO: 496 - protein, 11 aa, synthetic construct
  REGION 1..11: note = Heavy chain CDR2 sequence mut
QIYPQDGETKY

SEQ ID NO: 497 - protein, 11 aa, synthetic construct
  REGION 1..11: note = Heavy chain CDR2 sequence mut
QIYPRDGETKY

SEQ ID NO: 498 - protein, 11 aa, synthetic construct
  REGION 1..11: note = Heavy chain CDR2 sequence mut
QIYPSDGETKY

SEQ ID NO: 499 - protein, 11 aa, synthetic construct
  REGION 1..11: note = Heavy chain CDR2 sequence mut
QIYPTDGETKY

SEQ ID NO: 500 - protein, 11 aa, synthetic construct
  REGION 1..11: note = Heavy chain CDR2 sequence mut
QIYPVDGETKY

SEQ ID NO: 501 - protein, 11 aa, synthetic construct
  REGION 1..11: note = Heavy chain CDR2 sequence mut
QIYPWDGETKY

SEQ ID NO: 502 - protein, 11 aa, synthetic construct
  REGION 1..11: note = Heavy chain CDR2 sequence mut
QIYPYDGETKY

SEQ ID NO: 503 - protein, 11 aa, synthetic construct
  REGION 1..11: note = Heavy chain CDR2 sequence mut
QIYPGIGETKY

SEQ ID NO: 504 - protein, 11 aa, synthetic construct
  REGION 1..11: note = Heavy chain CDR2 sequence mut
QIYPGLGETKY

SEQ ID NO: 505 - protein, 11 aa, synthetic construct
  REGION 1..11: note = Heavy chain CDR2 sequence mut
QIYPGMGETKY

SEQ ID NO: 506 - protein, 11 aa, synthetic construct
  REGION 1..11: note = Heavy chain CDR2 sequence mut
QIYPGNGETKY

SEQ ID NO: 507 - protein, 11 aa, synthetic construct
  REGION 1..11: note = Heavy chain CDR2 sequence mut
QIYPGPGETKY

SEQ ID NO: 508 - protein, 11 aa, synthetic construct
  REGION 1..11: note = Heavy chain CDR2 sequence mut
QIYPGQGETKY

SEQ ID NO: 509 - protein, 11 aa, synthetic construct
  REGION 1..11: note = Heavy chain CDR2 sequence mut
QIYPGVGETKY

SEQ ID NO: 510 - protein, 11 aa, synthetic construct
  REGION 1..11: note = Heavy chain CDR2 sequence mut
QIYPGWGETKY

SEQ ID NO: 511 - protein, 11 aa, synthetic construct
  REGION 1..11: note = Heavy chain CDR2 sequence mut
QIYPGYGETKY

SEQ ID NO: 512 - protein, 11 aa, synthetic construct
  REGION 1..11: note = Heavy chain CDR2 sequence mut
QIYPGDAETKY

SEQ ID NO: 513 - protein, 11 aa, synthetic construct
  REGION 1..11: note = Heavy chain CDR2 sequence mut
QIYPGDDETKY

SEQ ID NO: 514 - protein, 11 aa, synthetic construct
  REGION 1..11: note = Heavy chain CDR2 sequence mut
QIYPGDEETKY

SEQ ID NO: 515 - protein, 11 aa, synthetic construct
  REGION 1..11: note = Heavy chain CDR2 sequence mut
QIYPGDFETKY

SEQ ID NO: 516 - protein, 11 aa, synthetic construct
  REGION 1..11: note = Heavy chain CDR2 sequence mut
QIYPGDHETKY

SEQ ID NO: 517 - protein, 11 aa, synthetic construct
  REGION 1..11: note = Heavy chain CDR2 sequence mut
QIYPGDIETKY

SEQ ID NO: 518 - protein, 11 aa, synthetic construct
  REGION 1..11: note = Heavy chain CDR2 sequence mut
QIYPGDKETKY

SEQ ID NO: 519 - protein, 11 aa, synthetic construct
  REGION 1..11: note = Heavy chain CDR2 sequence mut
QIYPGDLETKY

SEQ ID NO: 520 - protein, 11 aa, synthetic construct
  REGION 1..11: note = Heavy chain CDR2 sequence mut
QIYPGDMETKY

SEQ ID NO: 521 - protein, 11 aa, synthetic construct
  REGION 1..11: note = Heavy chain CDR2 sequence mut
QIYPGDNETKY

SEQ ID NO: 522 - protein, 11 aa, synthetic construct
  REGION 1..11: note = Heavy chain CDR2 sequence mut
QIYPGDPETKY

SEQ ID NO: 523 - protein, 11 aa, synthetic construct
  REGION 1..11: note = Heavy chain CDR2 sequence mut
QIYPGDQETKY

SEQ ID NO: 524 - protein, 11 aa, synthetic construct
  REGION 1..11: note = Heavy chain CDR2 sequence mut
QIYPGDRETKY

SEQ ID NO: 525 - protein, 11 aa, synthetic construct
  REGION 1..11: note = Heavy chain CDR2 sequence mut
QIYPGDSETKY

SEQ ID NO: 526 - protein, 11 aa, synthetic construct
  REGION 1..11: note = Heavy chain CDR2 sequence mut
QIYPGDTETKY

SEQ ID NO: 527 - protein, 11 aa, synthetic construct
  REGION 1..11: note = Heavy chain CDR2 sequence mut
QIYPGDVETKY

SEQ ID NO: 528 - protein, 11 aa, synthetic construct
  REGION 1..11: note = Heavy chain CDR2 sequence mut
QIYPGDYETKY

SEQ ID NO: 529 - protein, 11 aa, synthetic construct
  REGION 1..11: note = Heavy chain CDR2 sequence mut
QIYPGDGATKY

SEQ ID NO: 530 - protein, 11 aa, synthetic construct
  REGION 1..11: note = Heavy chain CDR2 sequence mut
QIYPGDGDTKY

SEQ ID NO: 531 - protein, 11 aa, synthetic construct
  REGION 1..11: note = Heavy chain CDR2 sequence mut
QIYPGDGMTKY

SEQ ID NO: 532 - protein, 11 aa, synthetic construct
  REGION 1..11: note = Heavy chain CDR2 sequence mut
QIYPGDGQTKY

SEQ ID NO: 533 - protein, 11 aa, synthetic construct
  REGION 1..11: note = Heavy chain CDR2 sequence mut
QIYPGDGVTKY

SEQ ID NO: 534 - protein, 11 aa, synthetic construct
  REGION 1..11: note = Heavy chain CDR2 sequence mut
QIYPGDGYTKY

SEQ ID NO: 535 - protein, 11 aa, synthetic construct
  REGION 1..11: note = Heavy chain CDR2 sequence mut
QIYPGDGEAKY

SEQ ID NO: 536 - protein, 11 aa, synthetic construct
  REGION 1..11: note = Heavy chain CDR2 sequence mut
QIYPGDGEDKY

SEQ ID NO: 537 - protein, 11 aa, synthetic construct
  REGION 1..11: note = Heavy chain CDR2 sequence mut
QIYPGDGEEKY

SEQ ID NO: 538 - protein, 11 aa, synthetic construct
  REGION 1..11: note = Heavy chain CDR2 sequence mut
QIYPGDGEFKY

SEQ ID NO: 539 - protein, 11 aa, synthetic construct
  REGION 1..11: note = Heavy chain CDR2 sequence mut
QIYPGDGEGKY

SEQ ID NO: 540 - protein, 11 aa, synthetic construct
  REGION 1..11: note = Heavy chain CDR2 sequence mut
QIYPGDGEHKY

SEQ ID NO: 541 - protein, 11 aa, synthetic construct
  REGION 1..11: note = Heavy chain CDR2 sequence mut
QIYPGDGEIKY

SEQ ID NO: 542 - protein, 11 aa, synthetic construct
  REGION 1..11: note = Heavy chain CDR2 sequence mut
QIYPGDGEKKY

SEQ ID NO: 543 - protein, 11 aa, synthetic construct
  REGION 1..11: note = Heavy chain CDR2 sequence mut
QIYPGDGEQKY

SEQ ID NO: 544 - protein, 11 aa, synthetic construct
  REGION 1..11: note = Heavy chain CDR2 sequence mut
QIYPGDGERKY

SEQ ID NO: 545 - protein, 11 aa, synthetic construct
  REGION 1..11: note = Heavy chain CDR2 sequence mut
QIYPGDGESKY

SEQ ID NO: 546 - protein, 11 aa, synthetic construct
  REGION 1..11: note = Heavy chain CDR2 sequence mut
QIYPGDGETKY

SEQ ID NO: 547 - protein, 11 aa, synthetic construct
  REGION 1..11: note = Heavy chain CDR2 sequence mut
QIYPGDGEVKY

SEQ ID NO: 548 - protein, 11 aa, synthetic construct
  REGION 1..11: note = Heavy chain CDR2 sequence mut
QIYPGDGEYKY

SEQ ID NO: 549 - protein, 11 aa, synthetic construct
  REGION 1..11: note = Heavy chain CDR2 sequence mut
QIYPGDGETDY

SEQ ID NO: 550 - protein, 11 aa, synthetic construct
  REGION 1..11: note = Heavy chain CDR2 sequence mut
QIYPGDGETEY

SEQ ID NO: 551 - protein, 11 aa, synthetic construct
  REGION 1..11: note = Heavy chain CDR2 sequence mut
QIYPGDGETHY

SEQ ID NO: 552 - protein, 11 aa, synthetic construct
  REGION 1..11: note = Heavy chain CDR2 sequence mut
QIYPGDGETIY

SEQ ID NO: 553 - protein, 11 aa, synthetic construct
  REGION 1..11: note = Heavy chain CDR2 sequence mut
QIYPGDGETLY

SEQ ID NO: 554 - protein, 11 aa, synthetic construct
  REGION 1..11: note = Heavy chain CDR2 sequence mut
QIYPGDGETMY

SEQ ID NO: 555 - protein, 11 aa, synthetic construct
  REGION 1..11: note = Heavy chain CDR2 sequence mut
QIYPGDGETNY

SEQ ID NO: 556 - protein, 11 aa, synthetic construct
  REGION 1..11: note = Heavy chain CDR2 sequence mut
QIYPGDGETPY

SEQ ID NO: 557 - protein, 11 aa, synthetic construct
  REGION 1..11: note = Heavy chain CDR2 sequence mut
QIYPGDGETQY

SEQ ID NO: 558 - protein, 11 aa, synthetic construct
  REGION 1..11: note = Heavy chain CDR2 sequence mut
QIYPGDGETRY

SEQ ID NO: 559 - protein, 11 aa, synthetic construct
  REGION 1..11: note = Heavy chain CDR2 sequence mut
QIYPGDGETSY

SEQ ID NO: 560 - protein, 11 aa, synthetic construct
  REGION 1..11: note = Heavy chain CDR2 sequence mut
QIYPGDGETTY

SEQ ID NO: 561 - protein, 11 aa, synthetic construct
  REGION 1..11: note = Heavy chain CDR2 sequence mut
QIYPGDGETVY

SEQ ID NO: 562 - protein, 11 aa, synthetic construct
  REGION 1..11: note = Heavy chain CDR2 sequence mut
QIYPGDGETWY

SEQ ID NO: 563 - protein, 11 aa, synthetic construct
  REGION 1..11: note = Heavy chain CDR2 sequence mut
QIYPGDGETYY

SEQ ID NO: 564 - protein, 11 aa, synthetic construct
  REGION 1..11: note = Heavy chain CDR2 sequence mut
QIYPGDGETKC

SEQ ID NO: 565 - protein, 11 aa, synthetic construct
  REGION 1..11: note = Heavy chain CDR2 sequence mut
QIYPGDGETKD

SEQ ID NO: 566 - protein, 11 aa, synthetic construct
  REGION 1..11: note = Heavy chain CDR2 sequence mut
QIYPGDGETKE

SEQ ID NO: 567 - protein, 11 aa, synthetic construct
  REGION 1..11: note = Heavy chain CDR2 sequence mut
QIYPGDGETKF

SEQ ID NO: 568 - protein, 11 aa, synthetic construct
  REGION 1..11: note = Heavy chain CDR2 sequence mut
QIYPGDGETKG

SEQ ID NO: 569 - protein, 11 aa, synthetic construct
  REGION 1..11: note = Heavy chain CDR2 sequence mut
QIYPGDGETKH

SEQ ID NO: 570 - protein, 11 aa, synthetic construct
  REGION 1..11: note = Heavy chain CDR2 sequence mut
QIYPGDGETKI

SEQ ID NO: 571 - protein, 11 aa, synthetic construct
  REGION 1..11: note = Heavy chain CDR2 sequence mut
QIYPGDGETKL

SEQ ID NO: 572 - protein, 11 aa, synthetic construct
  REGION 1..11: note = Heavy chain CDR2 sequence mut
QIYPGDGETKN

SEQ ID NO: 573 - protein, 11 aa, synthetic construct
  REGION 1..11: note = Heavy chain CDR2 sequence mut
QIYPGDGETKP

SEQ ID NO: 574 - protein, 11 aa, synthetic construct
  REGION 1..11: note = Heavy chain CDR2 sequence mut
QIYPGDGETKQ

SEQ ID NO: 575 - protein, 11 aa, synthetic construct
  REGION 1..11: note = Heavy chain CDR2 sequence mut
QIYPGDGETKR

SEQ ID NO: 576 - protein, 11 aa, synthetic construct
  REGION 1..11: note = Heavy chain CDR2 sequence mut
QIYPGDGETKT

SEQ ID NO: 577 - protein, 11 aa, synthetic construct
  REGION 1..11: note = Heavy chain CDR2 sequence mut
QIYPGDGETKW

SEQ ID NO: 578 - protein, 8 aa, synthetic construct
  REGION 1..8: note = NHGNYLPY
NHGNYLPY

SEQ ID NO: 579 - protein, 8 aa, synthetic construct
  REGION 1..8: note = THGNYLPY
THGNYLPY

SEQ ID NO: 580 - protein, 8 aa, synthetic construct
  REGION 1..8: note = Heavy chain CDR3 sequence mut
YHGNYLPY

SEQ ID NO: 581 - protein, 8 aa, synthetic construct
  REGION 1..8: note = Heavy chain CDR3 sequence mut
SHGNALPY

SEQ ID NO: 582 - protein, 8 aa, synthetic construct
  REGION 1..8: note = Heavy chain CDR3 sequence mut
SHGNCLPY

SEQ ID NO: 583 - protein, 8 aa, synthetic construct
  REGION 1..8: note = Heavy chain CDR3 sequence mut
SHGNFLPY

SEQ ID NO: 584 - protein, 8 aa, synthetic construct
  REGION 1..8: note = Heavy chain CDR3 sequence mut
SHGNHLPY

SEQ ID NO: 585 - protein, 8 aa, synthetic construct
  REGION 1..8: note = Heavy chain CDR3 sequence mut
SHGNNLPY

SEQ ID NO: 586 - protein, 8 aa, synthetic construct
  REGION 1..8: note = Heavy chain CDR3 sequence mut
SHGNSLPY

SEQ ID NO: 587 - protein, 8 aa, synthetic construct
  REGION 1..8: note = Heavy chain CDR3 sequence mut
SHGNYDPY

SEQ ID NO: 588 - protein, 8 aa, synthetic construct
  REGION 1..8: note = Heavy chain CDR3 sequence mut
SHGNYEPY

SEQ ID NO: 589 - protein, 8 aa, synthetic construct
  REGION 1..8: note = Heavy chain CDR3 sequence mut
SHGNYFPY

SEQ ID NO: 590 - protein, 8 aa, synthetic construct
  REGION 1..8: note = Heavy chain CDR3 sequence mut
SHGNYGPY

SEQ ID NO: 591 - protein, 8 aa, synthetic construct
  REGION 1..8: note = Heavy chain CDR3 sequence mut
SHGNYHPY

SEQ ID NO: 592 - protein, 8 aa, synthetic construct
  REGION 1..8: note = Heavy chain CDR3 sequence mut
SHGNYIPY

SEQ ID NO: 593 - protein, 8 aa, synthetic construct
  REGION 1..8: note = Heavy chain CDR3 sequence mut
SHGNYKPY

SEQ ID NO: 594 - protein, 8 aa, synthetic construct
  REGION 1..8: note = Heavy chain CDR3 sequence mut
SHGNYMPY

SEQ ID NO: 595 - protein, 8 aa, synthetic construct
  REGION 1..8: note = Heavy chain CDR3 sequence mut
SHGNYNPY

SEQ ID NO: 596 - protein, 8 aa, synthetic construct
  REGION 1..8: note = Heavy chain CDR3 sequence mut
SHGNYQPY

SEQ ID NO: 597 - protein, 8 aa, synthetic construct
  REGION 1..8: note = Heavy chain CDR3 sequence mut
SHGNYSPY

SEQ ID NO: 598 - protein, 8 aa, synthetic construct
  REGION 1..8: note = Heavy chain CDR3 sequence mut
SHGNYWPY

SEQ ID NO: 599 - protein, 8 aa, synthetic construct
  REGION 1..8: note = Heavy chain CDR3 sequence mut
SHGNYYPY

SEQ ID NO: 600 - protein, 8 aa, synthetic construct
  REGION 1..8: note = Heavy chain CDR3 sequence mut
SHGNYLAY

SEQ ID NO: 601 - protein, 8 aa, synthetic construct
  REGION 1..8: note = Heavy chain CDR3 sequence mut
SHGNYLEY

SEQ ID NO: 602 - protein, 8 aa, synthetic construct
  REGION 1..8: note = Heavy chain CDR3 sequence mut
SHGNYLFY

SEQ ID NO: 603 - protein, 8 aa, synthetic construct
  REGION 1..8: note = Heavy chain CDR3 sequence mut
SHGNYLGY

SEQ ID NO: 604 - protein, 8 aa, synthetic construct
  REGION 1..8: note = Heavy chain CDR3 sequence mut
SHGNYLHY

SEQ ID NO: 605 - protein, 8 aa, synthetic construct
  REGION 1..8: note = Heavy chain CDR3 sequence mut
SHGNYLKY

SEQ ID NO: 606 - protein, 8 aa, synthetic construct
  REGION 1..8: note = Heavy chain CDR3 sequence mut
SHGNYLLY

SEQ ID NO: 607 - protein, 8 aa, synthetic construct
  REGION 1..8: note = Heavy chain CDR3 sequence mut
SHGNYLQY

SEQ ID NO: 608 - protein, 8 aa, synthetic construct
  REGION 1..8: note = Heavy chain CDR3 sequence mut
SHGNYLRY

SEQ ID NO: 609 - protein, 8 aa, synthetic construct
  REGION 1..8: note = Heavy chain CDR3 sequence mut
SHGNYLSY

SEQ ID NO: 610 - protein, 8 aa, synthetic construct
  REGION 1..8: note = Heavy chain CDR3 sequence mut
SHGNYLVY

SEQ ID NO: 611 - protein, 8 aa, synthetic construct
  REGION 1..8: note = Heavy chain CDR3 sequence mut
SHGNYLWY

SEQ ID NO: 612 - protein, 8 aa, synthetic construct
  REGION 1..8: note = Heavy chain CDR3 sequence mut
SHGNYLYY

SEQ ID NO: 613 - protein, 8 aa, synthetic construct
  REGION 1..8: note = Heavy chain CDR3 sequence mut
SHGNYLPA

SEQ ID NO: 614 - protein, 8 aa, synthetic construct
  REGION 1..8: note = Heavy chain CDR3 sequence mut
SHGNYLPD

SEQ ID NO: 615 - protein, 8 aa, synthetic construct
  REGION 1..8: note = Heavy chain CDR3 sequence mut
SHGNYLPE

SEQ ID NO: 616 - protein, 8 aa, synthetic construct
  REGION 1..8: note = Heavy chain CDR3 sequence mut
SHGNYLPF

SEQ ID NO: 617 - protein, 8 aa, synthetic construct
  REGION 1..8: note = Heavy chain CDR3 sequence mut
SHGNYLPG

SEQ ID NO: 618 - protein, 8 aa, synthetic construct
  REGION 1..8: note = Heavy chain CDR3 sequence mut
SHGNYLPH

SEQ ID NO: 619 - protein, 8 aa, synthetic construct
  REGION 1..8: note = Heavy chain CDR3 sequence mut
SHGNYLPI

SEQ ID NO: 620 - protein, 8 aa, synthetic construct
  REGION 1..8: note = Heavy chain CDR3 sequence mut
SHGNYLPK

SEQ ID NO: 621 - protein, 8 aa, synthetic construct
  REGION 1..8: note = Heavy chain CDR3 sequence mut
SHGNYLPL

SEQ ID NO: 622 - protein, 8 aa, synthetic construct
  REGION 1..8: note = Heavy chain CDR3 sequence mut
SHGNYLPM

SEQ ID NO: 623 - protein, 8 aa, synthetic construct
  REGION 1..8: note = Heavy chain CDR3 sequence mut
SHGNYLPR

SEQ ID NO: 624 - protein, 8 aa, synthetic construct
  REGION 1..8: note = Heavy chain CDR3 sequence mut
SHGNYLPS

SEQ ID NO: 625 - protein, 8 aa, synthetic construct
  REGION 1..8: note = Heavy chain CDR3 sequence mut
SHGNYLPV

SEQ ID NO: 626 - protein, 5 aa, synthetic construct
  REGION 1..5: note = Light chain CDR1 sequence mut
ASYLH

SEQ ID NO: 627 - protein, 5 aa, synthetic construct
  REGION 1..5: note = Light chain CDR1 sequence mut
DSYLH

SEQ ID NO: 628 - protein, 5 aa, synthetic construct
  REGION 1..5: note = Light chain CDR1 sequence mut
FSYLH

SEQ ID NO: 629 - protein, 5 aa, synthetic construct
  REGION 1..5: note = Light chain CDR1 sequence mut
LSYLH

SEQ ID NO: 630 - protein, 5 aa, synthetic construct
  REGION 1..5: note = Light chain CDR1 sequence mut
MSYLH

SEQ ID NO: 631 - protein, 5 aa, synthetic construct
  REGION 1..5: note = Light chain CDR1 sequence mut
RSYLH

SEQ ID NO: 632 - protein, 5 aa, synthetic construct
  REGION 1..5: note = Light chain CDR1 sequence mut
TSYLH

SEQ ID NO: 633 - protein, 5 aa, synthetic construct
  REGION 1..5: note = Light chain CDR1 sequence mut
VSYLH

SEQ ID NO: 634 - protein, 5 aa, synthetic construct
  REGION 1..5: note = Light chain CDR1 sequence mut
WSYLH

SEQ ID NO: 635 - protein, 5 aa, synthetic construct
  REGION 1..5: note = Light chain CDR1 sequence mut
YSYLH

SEQ ID NO: 636 - protein, 5 aa, synthetic construct
  REGION 1..5: note = Light chain CDR1 sequence mut
SAYLH

SEQ ID NO: 637 - protein, 5 aa, synthetic construct
  REGION 1..5: note = Light chain CDR1 sequence mut
SDYLH

SEQ ID NO: 638 - protein, 5 aa, synthetic construct
  REGION 1..5: note = Light chain CDR1 sequence mut
SEYLH

SEQ ID NO: 639 - protein, 5 aa, synthetic construct
  REGION 1..5: note = Light chain CDR1 sequence mut
SFYLH

SEQ ID NO: 640 - protein, 5 aa, synthetic construct
  REGION 1..5: note = Light chain CDR1 sequence mut
SGYLH

SEQ ID NO: 641 - protein, 5 aa, synthetic construct
  REGION 1..5: note = Light chain CDR1 sequence mut
SHYLH

SEQ ID NO: 642 - protein, 5 aa, synthetic construct
  REGION 1..5: note = Light chain CDR1 sequence mut
SKYLH

SEQ ID NO: 643 - protein, 5 aa, synthetic construct
  REGION 1..5: note = Light chain CDR1 sequence mut
SMYLH

SEQ ID NO: 644 - protein, 5 aa, synthetic construct
  REGION 1..5: note = Light chain CDR1 sequence mut
SNYLH

SEQ ID NO: 645 - protein, 5 aa, synthetic construct
  REGION 1..5: note = Light chain CDR1 sequence mut
SPYLH

SEQ ID NO: 646 - protein, 5 aa, synthetic construct
  REGION 1..5: note = Light chain CDR1 sequence mut
SQYLH

SEQ ID NO: 647 - protein, 5 aa, synthetic construct
  REGION 1..5: note = Light chain CDR1 sequence mut
SVYLH

SEQ ID NO: 648 - protein, 5 aa, synthetic construct
  REGION 1..5: note = Light chain CDR1 sequence mut
SWYLH

SEQ ID NO: 649 - protein, 5 aa, synthetic construct
  REGION 1..5: note = Light chain CDR1 sequence mut
SSFLH

SEQ ID NO: 650 - protein, 5 aa, synthetic construct
  REGION 1..5: note = Light chain CDR1 sequence mut
SSYFH

SEQ ID NO: 651 - protein, 5 aa, synthetic construct
  REGION 1..5: note = Light chain CDR1 sequence mut
SSYHH

SEQ ID NO: 652 - protein, 5 aa, synthetic construct
  REGION 1..5: note = Light chain CDR1 sequence mut
SSYIH

SEQ ID NO: 653 - protein, 5 aa, synthetic construct
  REGION 1..5: note = Light chain CDR1 sequence mut
SSYKH

SEQ ID NO: 654 - protein, 5 aa, synthetic construct
  REGION 1..5: note = Light chain CDR1 sequence mut
SSYNH

SEQ ID NO: 655 - protein, 5 aa, synthetic construct
  REGION 1..5: note = Light chain CDR1 sequence mut
SSYPH

SEQ ID NO: 656 - protein, 5 aa, synthetic construct
  REGION 1..5: note = Light chain CDR1 sequence mut
SSYQH

SEQ ID NO: 657 - protein, 5 aa, synthetic construct
  REGION 1..5: note = Light chain CDR1 sequence mut
SSYRH

SEQ ID NO: 658 - protein, 5 aa, synthetic construct
  REGION 1..5: note = Light chain CDR1 sequence mut
SSYSH

SEQ ID NO: 659 - protein, 5 aa, synthetic construct
  REGION 1..5: note = Light chain CDR1 sequence mut
SSYTH

SEQ ID NO: 660 - protein, 5 aa, synthetic construct
  REGION 1..5: note = Light chain CDR1 sequence mut
SSYWH

SEQ ID NO: 661 - protein, 5 aa, synthetic construct
  REGION 1..5: note = Light chain CDR1 sequence mut
SSYYH

SEQ ID NO: 662 - protein, 7 aa, synthetic construct
  REGION 1..7: note = Light chain CDR2 sequence mut
ATSNLAS

SEQ ID NO: 663 - protein, 7 aa, synthetic construct
  REGION 1..7: note = Light chain CDR2 sequence mut
HTSNLAS

SEQ ID NO: 664 - protein, 7 aa, synthetic construct
  REGION 1..7: note = Light chain CDR2 sequence mut
NTSNLAS

SEQ ID NO: 665 - protein, 7 aa, synthetic construct
  REGION 1..7: note = Light chain CDR2 sequence mut
RTSNLAS

SEQ ID NO: 666 - protein, 7 aa, synthetic construct
  REGION 1..7: note = Light chain CDR2 sequence mut
SASNLAS

SEQ ID NO: 667 - protein, 7 aa, synthetic construct
  REGION 1..7: note = Light chain CDR2 sequence mut
SDSNLAS

SEQ ID NO: 668 - protein, 7 aa, synthetic construct
  REGION 1..7: note = Light chain CDR2 sequence mut
SFSNLAS

SEQ ID NO: 669 - protein, 7 aa, synthetic construct
  REGION 1..7: note = Light chain CDR2 sequence mut
SGSNLAS

SEQ ID NO: 670 - protein, 7 aa, synthetic construct
  REGION 1..7: note = Light chain CDR2 sequence mut
SHSNLAS

SEQ ID NO: 671 - protein, 7 aa, synthetic construct
  REGION 1..7: note = Light chain CDR2 sequence mut
SLSNLAS

SEQ ID NO: 672 - protein, 7 aa, synthetic construct
  REGION 1..7: note = Light chain CDR2 sequence mut
SMSNLAS

SEQ ID NO: 673 - protein, 7 aa, synthetic construct
  REGION 1..7: note = Light chain CDR2 sequence mut
SSSNLAS

SEQ ID NO: 674 - protein, 7 aa, synthetic construct
  REGION 1..7: note = Light chain CDR2 sequence mut
SVSNLAS

SEQ ID NO: 675 - protein, 7 aa, synthetic construct
  REGION 1..7: note = Light chain CDR2 sequence mut
SWSNLAS

SEQ ID NO: 676 - protein, 7 aa, synthetic construct
  REGION 1..7: note = Light chain CDR2 sequence mut
SYSNLAS

SEQ ID NO: 677 - protein, 7 aa, synthetic construct
  REGION 1..7: note = Light chain CDR2 sequence mut
STDNLAS

SEQ ID NO: 678 - protein, 7 aa, synthetic construct
  REGION 1..7: note = Light chain CDR2 sequence mut
STFNLAS

SEQ ID NO: 679 - protein, 7 aa, synthetic construct
  REGION 1..7: note = Light chain CDR2 sequence mut
STHNLAS

SEQ ID NO: 680 - protein, 7 aa, synthetic construct
  REGION 1..7: note = Light chain CDR2 sequence mut
STINLAS

SEQ ID NO: 681 - protein, 7 aa, synthetic construct
  REGION 1..7: note = Light chain CDR2 sequence mut
STKNLAS

SEQ ID NO: 682 - protein, 7 aa, synthetic construct
  REGION 1..7: note = Light chain CDR2 sequence mut
STLNLAS

SEQ ID NO: 683 - protein, 7 aa, synthetic construct
  REGION 1..7: note = Light chain CDR2 sequence mut
STNNLAS

SEQ ID NO: 684 - protein, 7 aa, synthetic construct
  REGION 1..7: note = Light chain CDR2 sequence mut
STQNLAS

SEQ ID NO: 685 - protein, 7 aa, synthetic construct
  REGION 1..7: note = Light chain CDR2 sequence mut
STYNLAS

SEQ ID NO: 686 - protein, 7 aa, synthetic construct
  REGION 1..7: note = Light chain CDR2 sequence mut
STSDLAS

SEQ ID NO: 687 - protein, 7 aa, synthetic construct
  REGION 1..7: note = Light chain CDR2 sequence mut
STSELAS

SEQ ID NO: 688 - protein, 7 aa, synthetic construct
  REGION 1..7: note = Light chain CDR2 sequence mut
STSFLAS

SEQ ID NO: 689 - protein, 7 aa, synthetic construct
  REGION 1..7: note = Light chain CDR2 sequence mut
STSGLAS

SEQ ID NO: 690 - protein, 7 aa, synthetic construct
  REGION 1..7: note = Light chain CDR2 sequence mut
STSHLAS

SEQ ID NO: 691 - protein, 7 aa, synthetic construct
  REGION 1..7: note = Light chain CDR2 sequence mut
STSILAS

SEQ ID NO: 692 - protein, 7 aa, synthetic construct
  REGION 1..7: note = Light chain CDR2 sequence mut
STSKLAS

SEQ ID NO: 693 - protein, 7 aa, synthetic construct
  REGION 1..7: note = Light chain CDR2 sequence mut
STSQLAS

SEQ ID NO: 694 - protein, 7 aa, synthetic construct
  REGION 1..7: note = Light chain CDR2 sequence mut
STSRLAS

SEQ ID NO: 695 - protein, 7 aa, synthetic construct
  REGION 1..7: note = Light chain CDR2 sequence mut
STSSLAS

SEQ ID NO: 696 - protein, 7 aa, synthetic construct
  REGION 1..7: note = Light chain CDR2 sequence mut
STSVLAS

SEQ ID NO: 697 - protein, 7 aa, synthetic construct
  REGION 1..7: note = Light chain CDR2 sequence mut
STSNDAS

SEQ ID NO: 698 - protein, 7 aa, synthetic construct
  REGION 1..7: note = Light chain CDR2 sequence mut
STSNEAS

SEQ ID NO: 699 - protein, 7 aa, synthetic construct
  REGION 1..7: note = Light chain CDR2 sequence mut
STSNFAS

SEQ ID NO: 700 - protein, 7 aa, synthetic construct
  REGION 1..7: note = Light chain CDR2 sequence mut
STSNGAS

SEQ ID NO: 701 - protein, 7 aa, synthetic construct
  REGION 1..7: note = Light chain CDR2 sequence mut
STSNHAS

SEQ ID NO: 702 - protein, 7 aa, synthetic construct
  REGION 1..7: note = Light chain CDR2 sequence mut
STSNIAS

SEQ ID NO: 703 - protein, 7 aa, synthetic construct
  REGION 1..7: note = Light chain CDR2 sequence mut
STSNKAS

SEQ ID NO: 704 - protein, 7 aa, synthetic construct
  REGION 1..7: note = Light chain CDR2 sequence mut
STSNMAS

SEQ ID NO: 705 - protein, 7 aa, synthetic construct
  REGION 1..7: note = Light chain CDR2 sequence mut
STSNNAS

SEQ ID NO: 706 - protein, 7 aa, synthetic construct
  REGION 1..7: note = Light chain CDR2 sequence mut
STSNPAS

SEQ ID NO: 707 - protein, 7 aa, synthetic construct
  REGION 1..7: note = Light chain CDR2 sequence mut
STSNQAS

SEQ ID NO: 708 - protein, 7 aa, synthetic construct
  REGION 1..7: note = Light chain CDR2 sequence mut
STSNVAS

SEQ ID NO: 709 - protein, 7 aa, synthetic construct
  REGION 1..7: note = Light chain CDR2 sequence mut
STSNWAS

SEQ ID NO: 710 - protein, 7 aa, synthetic construct
  REGION 1..7: note = Light chain CDR2 sequence mut
STSNYAS

SEQ ID NO: 711 - protein, 7 aa, synthetic construct
  REGION 1..7: note = Light chain CDR2 sequence mut
STSNLDS

SEQ ID NO: 712 - protein, 7 aa, synthetic construct
  REGION 1..7: note = Light chain CDR2 sequence mut
STSNLFS

SEQ ID NO: 713 - protein, 7 aa, synthetic construct
  REGION 1..7: note = Light chain CDR2 sequence mut
STSNLGS

SEQ ID NO: 714 - protein, 7 aa, synthetic construct
  REGION 1..7: note = Light chain CDR2 sequence mut
STSNLIS

SEQ ID NO: 715 - protein, 7 aa, synthetic construct
  REGION 1..7: note = Light chain CDR2 sequence mut
STSNLKS

SEQ ID NO: 716 - protein, 7 aa, synthetic construct
  REGION 1..7: note = Light chain CDR2 sequence mut
STSNLQS

SEQ ID NO: 717 - protein, 7 aa, synthetic construct
  REGION 1..7: note = Light chain CDR2 sequence mut
STSNLRS

SEQ ID NO: 718 - protein, 7 aa, synthetic construct
  REGION 1..7: note = Light chain CDR2 sequence mut
STSNLSS

SEQ ID NO: 719 - protein, 7 aa, synthetic construct
  REGION 1..7: note = Light chain CDR2 sequence mut
STSNLVS

SEQ ID NO: 720 - protein, 7 aa, synthetic construct
  REGION 1..7: note = Light chain CDR2 sequence mut
STSNLWS

SEQ ID NO: 721 - protein, 7 aa, synthetic construct
  REGION 1..7: note = Light chain CDR2 sequence mut
STSNLAA

SEQ ID NO: 722 - protein, 7 aa, synthetic construct
  REGION 1..7: note = Light chain CDR2 sequence mut
STSNLAD

SEQ ID NO: 723 - protein, 7 aa, synthetic construct
  REGION 1..7: note = Light chain CDR2 sequence mut
STSNLAF

SEQ ID NO: 724 - protein, 7 aa, synthetic construct
  REGION 1..7: note = Light chain CDR2 sequence mut
STSNLAG

SEQ ID NO: 725 - protein, 7 aa, synthetic construct
  REGION 1..7: note = Light chain CDR2 sequence mut
STSNLAH

SEQ ID NO: 726 - protein, 7 aa, synthetic construct
  REGION 1..7: note = Light chain CDR2 sequence mut
STSNLAK

SEQ ID NO: 727 - protein, 7 aa, synthetic construct
  REGION 1..7: note = Light chain CDR2 sequence mut
STSNLAL

SEQ ID NO: 728 - protein, 7 aa, synthetic construct
  REGION 1..7: note = Light chain CDR2 sequence mut
STSNLAR

SEQ ID NO: 729 - protein, 7 aa, synthetic construct
  REGION 1..7: note = Light chain CDR2 sequence mut
STSNLAT

SEQ ID NO: 730 - protein, 7 aa, synthetic construct
  REGION 1..7: note = Light chain CDR2 sequence mut
STSNLAV

SEQ ID NO: 731 - protein, 7 aa, synthetic construct
  REGION 1..7: note = Light chain CDR2 sequence mut
STSNLAW

SEQ ID NO: 732 - protein, 8 aa, synthetic construct
  REGION 1..8: note = Light chain CDR2 sequence
STSNLASG

SEQ ID NO: 733 - protein, 8 aa, synthetic construct
  REGION 1..8: note = Light chain CDR2 sequence mut
STSNLASA

SEQ ID NO: 734 - protein, 8 aa, synthetic construct
  REGION 1..8: note = Light chain CDR2 sequence mut
STSNLASF

SEQ ID NO: 735 - protein, 8 aa, synthetic construct
  REGION 1..8: note = Light chain CDR2 sequence mut
STSNLASI

SEQ ID NO: 736 - protein, 8 aa, synthetic construct
  REGION 1..8: note = Light chain CDR2 sequence mut
STSNLASN

SEQ ID NO: 737 - protein, 8 aa, synthetic construct
  REGION 1..8: note = Light chain CDR2 sequence mut
STSNLASP

SEQ ID NO: 738 - protein, 8 aa, synthetic construct
  REGION 1..8: note = Light chain CDR2 sequence mut
STSNLASR

SEQ ID NO: 739 - protein, 8 aa, synthetic construct
  REGION 1..8: note = Light chain CDR2 sequence mut
STSNLASS

SEQ ID NO: 740 - protein, 8 aa, synthetic construct
  REGION 1..8: note = Light chain CDR2 sequence mut
STSNLAST

SEQ ID NO: 741 - protein, 8 aa, synthetic construct
  REGION 1..8: note = Light chain CDR2 sequence mut
STSNLASV

SEQ ID NO: 742 - protein, 8 aa, synthetic construct
  REGION 1..8: note = Light chain CDR2 sequence mut
STSNLASY

SEQ ID NO: 743 - protein, 10 aa, synthetic construct
  REGION 1..10: note = Light chain CDR3 sequence mut
AQYHRSPPTF

SEQ ID NO: 744 - protein, 10 aa, synthetic construct
  REGION 1..10: note = Light chain CDR3 sequence mut
CQYHRSPPTF

SEQ ID NO: 745 - protein, 10 aa, synthetic construct
  REGION 1..10: note = Light chain CDR3 sequence mut
DQYHRSPPTF

SEQ ID NO: 746 - protein, 10 aa, synthetic construct
  REGION 1..10: note = Light chain CDR3 sequence mut
EQYHRSPPTF

SEQ ID NO: 747 - protein, 10 aa, synthetic construct
  REGION 1..10: note = Light chain CDR3 sequence mut
FQYHRSPPTF

SEQ ID NO: 748 - protein, 10 aa, synthetic construct
  REGION 1..10: note = Light chain CDR3 sequence mut
IQYHRSPPTF

SEQ ID NO: 749 - protein, 10 aa, synthetic construct
  REGION 1..10: note = Light chain CDR3 sequence mut
KQYHRSPPTF

SEQ ID NO: 750 - protein, 10 aa, synthetic construct
  REGION 1..10: note = Light chain CDR3 sequence mut
LQYHRSPPTF

SEQ ID NO: 751 - protein, 10 aa, synthetic construct
  REGION 1..10: note = Light chain CDR3 sequence mut
NQYHRSPPTF

SEQ ID NO: 752 - protein, 10 aa, synthetic construct
  REGION 1..10: note = Light chain CDR3 sequence mut
RQYHRSPPTF

SEQ ID NO: 753 - protein, 10 aa, synthetic construct
  REGION 1..10: note = Light chain CDR3 sequence mut
SQYHRSPPTF

SEQ ID NO: 754 - protein, 10 aa, synthetic construct
  REGION 1..10: note = Light chain CDR3 sequence mut
TQYHRSPPTF

SEQ ID NO: 755 - protein, 10 aa, synthetic construct
  REGION 1..10: note = Light chain CDR3 sequence mut
VQYHRSPPTF

SEQ ID NO: 756 - protein, 10 aa, synthetic construct
  REGION 1..10: note = Light chain CDR3 sequence mut
WQYHRSPPTF

SEQ ID NO: 757 - protein, 10 aa, synthetic construct
  REGION 1..10: note = Light chain CDR3 sequence mut
YQYHRSPPTF

SEQ ID NO: 758 - protein, 10 aa, synthetic construct
  REGION 1..10: note = Light chain CDR3 sequence mut
HAYHRSPPTF

SEQ ID NO: 759 - protein, 10 aa, synthetic construct
  REGION 1..10: note = Light chain CDR3 sequence mut
HCYHRSPPTF

SEQ ID NO: 760 - protein, 10 aa, synthetic construct
  REGION 1..10: note = Light chain CDR3 sequence mut
HEYHRSPPTF

SEQ ID NO: 761 - protein, 10 aa, synthetic construct
  REGION 1..10: note = Light chain CDR3 sequence mut
HGYHRSPPTF

SEQ ID NO: 762 - protein, 10 aa, synthetic construct
  REGION 1..10: note = Light chain CDR3 sequence mut
HNYHRSPPTF

SEQ ID NO: 763 - protein, 10 aa, synthetic construct
  REGION 1..10: note = Light chain CDR3 sequence mut
HSYHRSPPTF

SEQ ID NO: 764 - protein, 10 aa, synthetic construct
  REGION 1..10: note = Light chain CDR3 sequence mut
HTYHRSPPTF

SEQ ID NO: 765 - protein, 10 aa, synthetic construct
  REGION 1..10: note = Light chain CDR3 sequence mut
HVYHRSPPTF

SEQ ID NO: 766 - protein, 10 aa, synthetic construct
  REGION 1..10: note = Light chain CDR3 sequence mut
HQAHRSPPTF

SEQ ID NO: 767 - protein, 10 aa, synthetic construct
  REGION 1..10: note = Light chain CDR3 sequence mut
HQFHRSPPTF

SEQ ID NO: 768 - protein, 10 aa, synthetic construct
  REGION 1..10: note = Light chain CDR3 sequence mut
HQGHRSPPTF

SEQ ID NO: 769 - protein, 10 aa, synthetic construct
  REGION 1..10: note = Light chain CDR3 sequence mut
HQHHRSPPTF

SEQ ID NO: 770 - protein, 10 aa, synthetic construct
  REGION 1..10: note = Light chain CDR3 sequence mut
HQQHRSPPTF

SEQ ID NO: 771 - protein, 10 aa, synthetic construct
  REGION 1..10: note = Light chain CDR3 sequence mut
HQWHRSPPTF

SEQ ID NO: 772 - protein, 10 aa, synthetic construct
  REGION 1..10: note = Light chain CDR3 sequence mut
HQYARSPPTF

SEQ ID NO: 773 - protein, 10 aa, synthetic construct
  REGION 1..10: note = Light chain CDR3 sequence mut
HQYERSPPTF

SEQ ID NO: 774 - protein, 10 aa, synthetic construct
  REGION 1..10: note = Light chain CDR3 sequence mut
HQYGRSPPTF

SEQ ID NO: 775 - protein, 10 aa, synthetic construct
  REGION 1..10: note = Light chain CDR3 sequence mut
HQYKRSPPTF

SEQ ID NO: 776 - protein, 10 aa, synthetic construct
  REGION 1..10: note = Light chain CDR3 sequence mut
HQYLRSPPTF

SEQ ID NO: 777 - protein, 10 aa, synthetic construct
  REGION 1..10: note = Light chain CDR3 sequence mut
HQYNRSPPTF

SEQ ID NO: 778 - protein, 10 aa, synthetic construct
  REGION 1..10: note = Light chain CDR3 sequence mut
HQYQRSPPTF

SEQ ID NO: 779 - protein, 10 aa, synthetic construct
  REGION 1..10: note = Light chain CDR3 sequence mut
HQYSRSPPTF

SEQ ID NO: 780 - protein, 10 aa, synthetic construct
  REGION 1..10: note = Light chain CDR3 sequence mut
HQYTRSPPTF

SEQ ID NO: 781 - protein, 10 aa, synthetic construct
  REGION 1..10: note = Light chain CDR3 sequence mut
HQYVRSPPTF

SEQ ID NO: 782 - protein, 10 aa, synthetic construct
  REGION 1..10: note = Light chain CDR3 sequence mut
HQYWRSPPTF

SEQ ID NO: 783 - protein, 10 aa, synthetic construct
  REGION 1..10: note = Light chain CDR3 sequence mut
HQYHASPPTF

SEQ ID NO: 784 - protein, 10 aa, synthetic construct
  REGION 1..10: note = Light chain CDR3 sequence mut
HQYHCSPPTF

SEQ ID NO: 785 - protein, 10 aa, synthetic construct
  REGION 1..10: note = Light chain CDR3 sequence mut
HQYHDSPPTF

SEQ ID NO: 786 - protein, 10 aa, synthetic construct
  REGION 1..10: note = Light chain CDR3 sequence mut
HQYHESPPTF

SEQ ID NO: 787 - protein, 10 aa, synthetic construct
  REGION 1..10: note = Light chain CDR3 sequence mut
HQYHFSPPTF

SEQ ID NO: 788 - protein, 10 aa, synthetic construct
  REGION 1..10: note = Light chain CDR3 sequence mut
HQYHISPPTF

SEQ ID NO: 789 - protein, 10 aa, synthetic construct
  REGION 1..10: note = Light chain CDR3 sequence mut
HQYHLSPPTF

SEQ ID NO: 790 - protein, 10 aa, synthetic construct
  REGION 1..10: note = Light chain CDR3 sequence mut
HQYHMSPPTF

SEQ ID NO: 791 - protein, 10 aa, synthetic construct
  REGION 1..10: note = Light chain CDR3 sequence mut
HQYHNSPPTF

SEQ ID NO: 792 - protein, 10 aa, synthetic construct
  REGION 1..10: note = Light chain CDR3 sequence mut
HQYHQSPPTF

SEQ ID NO: 793 - protein, 10 aa, synthetic construct
  REGION 1..10: note = Light chain CDR3 sequence mut
HQYHVSPPTF

SEQ ID NO: 794 - protein, 10 aa, synthetic construct
  REGION 1..10: note = Light chain CDR3 sequence mut
HQYHWSPPTF

SEQ ID NO: 795 - protein, 10 aa, synthetic construct
  REGION 1..10: note = Light chain CDR3 sequence mut
HQYHYSPPTF

SEQ ID NO: 796 - protein, 10 aa, synthetic construct
  REGION 1..10: note = Light chain CDR3 sequence mut
HQYHRAPPTF

SEQ ID NO: 797 - protein, 10 aa, synthetic construct
  REGION 1..10: note = Light chain CDR3 sequence mut
HQYHRCPPTF

SEQ ID NO: 798 - protein, 10 aa, synthetic construct
  REGION 1..10: note = Light chain CDR3 sequence mut
HQYHREPPTF

SEQ ID NO: 799 - protein, 10 aa, synthetic construct
  REGION 1..10: note = Light chain CDR3 sequence mut
HQYHRFPPTF

SEQ ID NO: 800 - protein, 10 aa, synthetic construct
  REGION 1..10: note = Light chain CDR3 sequence mut
HQYHRGPPTF

SEQ ID NO: 801 - protein, 10 aa, synthetic construct
  REGION 1..10: note = Light chain CDR3 sequence mut
HQYHRIPPTF

SEQ ID NO: 802 - protein, 10 aa, synthetic construct
  REGION 1..10: note = Light chain CDR3 sequence mut
HQYHRMPPTF

SEQ ID NO: 803 - protein, 10 aa, synthetic construct
  REGION 1..10: note = Light chain CDR3 sequence mut
HQYHRPPPTF

SEQ ID NO: 804 - protein, 10 aa, synthetic construct
  REGION 1..10: note = Light chain CDR3 sequence mut
HQYHRRPPTF

SEQ ID NO: 805 - protein, 10 aa, synthetic construct
  REGION 1..10: note = Light chain CDR3 sequence mut
HQYHRTPPTF

SEQ ID NO: 806 - protein, 10 aa, synthetic construct
  REGION 1..10: note = Light chain CDR3 sequence mut
HQYHRVPPTF

SEQ ID NO: 807 - protein, 10 aa, synthetic construct
  REGION 1..10: note = Light chain CDR3 sequence mut
HQYHRWPPTF

SEQ ID NO: 808 - protein, 10 aa, synthetic construct
  REGION 1..10: note = Light chain CDR3 sequence mut
HQYHRYPPTF

SEQ ID NO: 809 - protein, 10 aa, synthetic construct
  REGION 1..10: note = Light chain CDR3 sequence mut
HQYHRSKPTF

SEQ ID NO: 810 - protein, 10 aa, synthetic construct
  REGION 1..10: note = Light chain CDR3 sequence mut
HQYHRSWPTF

SEQ ID NO: 811 - protein, 10 aa, synthetic construct
  REGION 1..10: note = Light chain CDR3 sequence mut
HQYHRSYPTF

SEQ ID NO: 812 - protein, 10 aa, synthetic construct
  REGION 1..10: note = Light chain CDR3 sequence mut
HQYHRSPDTF

SEQ ID NO: 813 - protein, 10 aa, synthetic construct
  REGION 1..10: note = Light chain CDR3 sequence mut
HQYHRSPHTF

SEQ ID NO: 814 - protein, 10 aa, synthetic construct
  REGION 1..10: note = Light chain CDR3 sequence mut
HQYHRSPPDF

SEQ ID NO: 815 - protein, 10 aa, synthetic construct
  REGION 1..10: note = Light chain CDR3 sequence mut
HQYHRSPPFF

SEQ ID NO: 816 - protein, 10 aa, synthetic construct
  REGION 1..10: note = Light chain CDR3 sequence mut
HQYHRSPPGF

SEQ ID NO: 817 - protein, 10 aa, synthetic construct
  REGION 1..10: note = Light chain CDR3 sequence mut
HQYHRSPPIF

SEQ ID NO: 818 - protein, 10 aa, synthetic construct
  REGION 1..10: note = Light chain CDR3 sequence mut
HQYHRSPPLF

SEQ ID NO: 819 - protein, 10 aa, synthetic construct
  REGION 1..10: note = Light chain CDR3 sequence mut
HQYHRSPPMF

SEQ ID NO: 820 - protein, 10 aa, synthetic construct
  REGION 1..10: note = Light chain CDR3 sequence mut
HQYHRSPPNF

SEQ ID NO: 821 - protein, 10 aa, synthetic construct
  REGION 1..10: note = Light chain CDR3 sequence mut
HQYHRSPPQF

SEQ ID NO: 822 - protein, 10 aa, synthetic construct
  REGION 1..10: note = Light chain CDR3 sequence mut
HQYHRSPPRF

SEQ ID NO: 823 - protein, 10 aa, synthetic construct
  REGION 1..10: note = Light chain CDR3 sequence mut
HQYHRSPPSF

SEQ ID NO: 824 - protein, 10 aa, synthetic construct
  REGION 1..10: note = Light chain CDR3 sequence mut
HQYHRSPPVF

SEQ ID NO: 825 - protein, 10 aa, synthetic construct
  REGION 1..10: note = Light chain CDR3 sequence mut
HQYHRSPPYF

SEQ ID NO: 826 - DNA, 50 bp, synthetic construct
  misc_feature 1..50: note = Apollo_ECL2_h2m_F
  source 1..50: mol_type = other DNA
ctgcaatctgtttgctcagacattttcccactcattgatgaaacctacct

SEQ ID NO: 827 - DNA, 50 bp, synthetic construct
  misc_feature 1..50: note = Apollo_ECL2_h2m_R
  source 1..50: mol_type = other DNA
ggaaaatgtctgagcaaacagattgcagtttcttgcagttccagcccagg

SEQ ID NO: 828 - protein, 215 aa, synthetic construct; identical to SEQ ID NO: 338
  REGION 1..215: note = P1C4 Light Chain Parental Sequence

SEQ ID NO: 829 - protein, 215 aa, synthetic construct
  REGION 1..215: note = P1C4 Clone RY-LC-B12__FACS3
EIVLTQSPATLSLSPGERATLSCRASQSVSSRYLHWYQQKPGQAPRLLIYSTSRLASGIPARFSGSGSGTDFTLTISRLEPEDFAVYYCQQYHRSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 830 - protein, 215 aa, synthetic construct
  REGION 1..215: note = P1C4 Clone RY-LC-A10__FACS5
EIVLTQSPATLSLSPGERATLSCRASQSVSSRYLHWYQQKPGQAPRLLIYSTSNRASGIPARFSGSGSGTDFTLTISRLEPEDFAVYYCQQYHRSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 831 - protein, 215 aa, synthetic construct
  REGION 1..215: note = P1C4 Clone RY-HL-LC-C12__FACS4
EIVLTQSPATLSLSPGERATLSCRASQSVSSRYLHWYQQKPGQAPRLLIYSTSRLASGIPARFSGSGSGTDFTLTISRLEPEDFAVYYCHQYHRSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 832 - protein, 215 aa, synthetic construct
  REGION 1..215: note = P1C4 Clone RY-HL-LC-A12__FACS4
EIVLTQSPATLSLSPGERATLSCRASQSVSSRYLHWYQQKPGQAPRLLIYGTSNLASGIPARFSGSGSGTDFTLTISRLEPEDFAVYYCHQYSRSPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 833 - protein, 5 aa, synthetic construct
  REGION 1..5: note = Light chain CDR1 sequence mut
SRYLH

SEQ ID NO: 834 - protein, 7 aa, synthetic construct
  REGION 1..7: note = Light chain CDR2 sequence mut
GTSNLAS

SEQ ID NO: 835 - protein, 7 aa, synthetic construct
  REGION 1..7: note = Light chain CDR2 sequence mut
STSNRAS

SEQ ID NO: 836 - protein, 10 aa, synthetic construct
  REGION 1..10: note = Light chain CDR3 sequence mut
QQYHRSPPTF